(19) United States
(12) Patent Application Publication
(10) Pub. No.: US 2026/0159823 A1
(43) Pub. Date: Jun. 11, 2026

(54) TYPE II CAS PROTEIN, CRISPR-CAS SYSTEM AND USES THEREOF

(71) Applicant: GENEDITBIO LIMITED, Hong Kong (CN)

(72) Inventor: Bang Wang, Hong Kong (CN)

(21) Appl. No.: 19/540,631

(22) Filed: Feb. 14, 2026

Related U.S. Application Data
(63) Continuation of application No. PCT/CN2024/112724, filed on Aug. 16, 2024.

(30) Foreign Application Priority Data
Aug. 16, 2023 (WO) PCT/CN2023/113355
Sep. 4, 2023 (WO) PCT/CN2023/116757
Dec. 6, 2023 (WO) PCT/CN2023/136724
May 6, 2024 (WO) PCT/CN2024/091198
May 6, 2024 (WO) PCT/CN2024/091203
May 6, 2024 (WO) PCT/CN2024/091211

(51) Int. Cl. C12N 9/22; C12N 15/11; C12N 15/90

(52) U.S. Cl. CPC C12N 9/226; C12N 15/111; C12N 15/90; C12N 2310/20

(57) ABSTRACT

The current disclosure pertains to Type II Cas proteins, CRISPR-Cas systems, and their various applications. The Type II Cas proteins outlined in this disclosure broaden the utility of CRISPR-Cas systems for targeting or modifying genes.

### CROSS-REFERENCE TOO RELATED APPLICATIONS

This application is a continuation application of PCT/CN2024/112724, filed on Aug. 16, 2024, which claims priority to PCT application PCT/CN2023/113355, filed on Aug. 16, 2023, PCT application PCT/CN2023/116757, filed on Sep. 4, 2023, PCT application PCT/CN2023/136724, filed on Dec. 6, 2023, PCT application PCT/CN2024/091203, filed on May 6, 2024, PCT application PCT/CN2024/091198, filed on May 6, 2024, and PCT application PCT/CN2024/091211, filed on May 6, 2024, the entire contents of which are hereby incorporated by reference.

### REFERENCE TO SEQUENCE LISTING

The instant application contains a Sequence Listing as an XML file entitled “BSP26410440US-CA-SEQ” created on Feb. 11, 2026 and having a size of 1,597,127 bytes. The Sequence Listing is hereby incorporated by reference.

### TECHNICAL FIELD

The present disclosure relates to type II Cas protein, CRISPR-Cas system and uses thereof. Particularly, the type II Cas protein and CRISPR-Cas system are used for gene targeting or gene editing.

### BACKGROUND

Targeted genome editing or modification is rapidly becoming an important tool for basic and applied research, with clustered regularly interspaced short palindromic repeats and CRISPR-associated proteins (CRISPR-Cas) system showing the most promising due to the ease of altering target specificity by engineering associated guide RNAs. Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications.

Various of CRISPR-Cas system had been explored and different CRISPR-Cas systems present different characteristics, like as CRISPR-Cas9 system which belongs to the class 2 CRISPR-Cas system, has been harnessed for genome editing and holds tremendous promise for biomedical research.

### SUMMARY

This invention provides an engineered, non-naturally occurring Type II CRISPR-associated (Cas) protein having a sequence identity of at least 70% to the amino acid sequence of any one of SEQ ID NOs: 1-71, or a variant thereof. In some embodiments, the Cas protein having a sequence identity of at least 75%, 80%, 85%, 90%, 92%, 95%, 98%, 99% or 100% to the amino acid sequence of any one of SEQ ID NOs: 1-71.

This invention also provides an engineered, non-naturally occurring Type II CRISPR-associated (Cas) protein, wherein the Cas protein has a sequence identity of at least 70%, 75%, 80%, 85%, 90%, 92%, 95%, 98%, 99%, or 100% to the amino acid sequence of any one of SEQ ID NOs: 1-71, with the exception of the amino acid “M” at position 1 of the sequence.

In some embodiments, the Cas protein further comprises an effector domain (or functional domain). Such effector domains can have one or more types of enzymatic activities, including polymerase activity, ligase activity, reverse transcriptase activity, deaminase activity, replication activity, or proofreading activity. In some embodiment, the effector domains domain comprises a nuclease, a nickase, a deaminase, a reverse transcriptase, a recombinase, a methyltransferase, a methylase, an acetylase, an acetyltransferase, a transcriptional activator, a transcriptional repressor domain, a cryptochrome, a light inducible/controllable domain, or a chemically inducible/controllable domain.

In some embodiments, the Cas protein further comprises one or more of a nuclear localization signal sequence, a nuclear export signal sequence, a cell penetrating peptide sequence, an affinity tag. the type II Cas protein comprises one or more nuclear localization signal(s) NLS(s). The NLS(s) can locate at the end or other portion of the peptide. The NLS(s) located each end or other portion of the Cas9 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas9 amino acid sequence comprising one NLS and the C-terminal end of the Cas9 amino acid sequence comprising one NLS. The amino acid sequence of NLS fused to the N-terminal end and/or the C-terminal end of the Cas9 amino acid sequence respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of C-terminal NLS is set forth in SEQ ID NO: 881 or 882. In some embodiments, the amino acid sequence of the C-terminal FLAG sequence is set forth in SEQ ID NO: 883. Other available sequences and different combinations can also be chosen for the NLSs sequences and FLAG sequence.

In some embodiments, the Cas protein, the Cas protein comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of any one of SEQ ID NOs: 1-71.

In some preferred embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 9, 12, 19, 21, 22, 24, 25, 27, 29, 30, 31, 36, 37, 38, 43, 44, 51, 56, 59, 60, and 68). In some other preferred embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 9, 12, 19, 21, 22, 24, 25, 27, 29, 30, 31, 36, 37, 38, 43, 44, 51, 56, 59, 60, and 68, with the exception of the amino acid “M” at position 1 of the sequence.

One of the key elements of the CRISPR-Cas system associated modification process is the Protospacer Adjacent Motif (PAM), a short DNA sequence immediately adjacent to the target DNA sequence. The PAM sequence is essential for Cas protein binding and cleavage activity, ensuring that only the intended DNA sequences are targeted for editing. The ability of the Cas protein to recognize these specific PAM sequences is due to its unique structural features and binding interactions with the DNA. Each PAM sequence provides a distinct motif that the Cas protein can identify, ensuring accurate and efficient targeting. For example, the NRRANH sequence may offer a specific arrangement of nucleotides that the Cas protein can bind to with high affinity. These diverse range of PAM sequences expands the potential applications of the CRISPR-Cas system. It enables researchers and scientists to target a wider variety of DNA sequences for editing, increasing the versatility and effectiveness of this powerful gene-editing tool. Additionally, understanding these PAM sequences can aid in the development of more advanced Cas proteins with even greater targeting capabilities, further advancing the field of gene editing and its potential benefits for research, medicine, and biotechnology. The Cas protein, as disclosed in this invention, exhibits a unique capability to recognize a diverse range of PAM sequences. These sequences include NRRANH, NRHACT, NRAAR, NNNCCY, NNRYYYY, NGG, NNNNCAA, NRNACN, NNGR, NGGNR, NNNCCH, NRRAAG, NRHRAC, NRYART, NRHACC, NRAAR, NRNVHH, YMACAW, NAHAA, NRHAYY, or NGGHA. This specific recognition of these Cas proteins allows for greater flexibility in selecting target DNA sequences for editing.

In some embodiments, the Cas protein, as disclosed in this invention, is capable of recognizing at least one protospacer adjacent motif (PAM) having or comprising a sequence of: NRRANH, NRHACT, NRAAR, NNNCCY, NNRYYYY, NGG, NNNNCAA, NRNACN, NNGR, NGGNR, NNNCCH, NRRAAG, NRHRAC, NRYART, NRHACC, NRAAR, NRNVHH, YMACAW, NAHAA, NRHAYY, or NGGHA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and is capable of recognizing a protospacer adjacent motif (PAM) having a sequence of: NRRANH. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and is capable of recognizing a protospacer adjacent motif (PAM) having a sequence of: NRHACT. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 19, and is capable of recognizing a protospacer adjacent motif (PAM) having a sequence of: NRAAR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 21, and is capable of recognizing a protospacer adjacent motif (PAM) having a sequence of: NNNCCY In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 22, and is capable of recognizing a protospacer adjacent motif (PAM) having a sequence of: NNRYYYY In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 24, and is capable of recognizing a protospacer adjacent motif (PAM) having a sequence of: NGG. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 25, and is capable of recognizing a protospacer adjacent motif (PAM) having a sequence of: NNNNCAA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 27, and is capable of recognizing a protospacer adjacent motif (PAM) having a sequence of: NRNACN. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 29, and is capable of recognizing a protospacer adjacent motif (PAM) having a sequence of: NNGR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 30, and is capable of recognizing a protospacer adjacent motif (PAM) having a sequence of: NGGNR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and is capable of recognizing a protospacer adjacent motif (PAM) having a sequence of: NNNCCH. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 36, and is capable of recognizing a protospacer adjacent motif (PAM) having a sequence of: NRRAAG. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 37, and is capable of recognizing a protospacer adjacent motif (PAM) having a sequence of: NRHRAC. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 38, and is capable of recognizing a protospacer adjacent motif (PAM) having a sequence of: NRYART. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 43, and is capable of recognizing a protospacer adjacent motif (PAM) having a sequence of: NRHACC. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 44, and is capable of recognizing a protospacer adjacent motif (PAM) having a sequence of: NRAAR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 51, and is capable of recognizing a protospacer adjacent motif (PAM) having a sequence of: NRNVHH. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 56, and is capable of recognizing a protospacer adjacent motif (PAM) having a sequence of: YMACAW. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 59, and is capable of recognizing a protospacer adjacent motif (PAM) having a sequence of: NAHAA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 60, and is capable of recognizing a protospacer adjacent motif (PAM) having a sequence of: NRHAYY. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 68, and is capable of recognizing a protospacer adjacent motif (PAM) having a sequence of: NGGHA.

In some embodiments, the Cas protein is a nickase or dead Cas protein. The DNA cleavage domain of an active Cas protein in this invention include two subdomains, the HNH nuclease subdomain and the RuvC subdomain. Mutations within these subdomains can silence the nuclease activity of the Cas protein. In some embodiments, Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and includes a mutation at residue D11 or H859; or has a sequence identity of at least 70%, 71%, 72%, 73%, 74%, 75%, 76%, 77%, 78%, 79%, 80%, 81%, 82%8, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and includes a mutation at D12 or H903. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and includes a mutation at residue D11 or H859, with the exception of the amino acid “M” at position 1 of the sequence; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862, with the exception of the amino acid “M” at position 1 of the sequence; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and includes a mutation at D12 or H903, with the exception of the amino acid “M” at position 1 of the sequence. In some embodiments, the mutation at residue D11 or H859 of SEQ ID NO: 9 is D11A or H859A; the mutation at residue D10 or H862 of SEQ ID NO: 12 is D10A or H862A; or the mutation at residue D12 or H903 of SEQ ID NO: 31 is D12A or H903A. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 869-877.

This invention also provides an engineered, non-naturally occurring polynucleotide encoding the Type II CRISPR-associated (Cas) protein as disclosed in this invention.

In some embodiments, the polynucleotide encoding the Cas protein is operably linked to a promoter and is presented in a vector; optionally, the vector is selected from the group consisting of: a retroviral vector, a lentiviral vector, a phage vector, an adenoviral vector, an adeno-associated virus vector, a herpes simplex virus vector and a plasmid vector.

In some embodiments, the polynucleotide is a ribonucleotide sequence or a deoxyribonucleotide sequence, or analogs thereof, optionally, the polynucleotide is codon-optimized for expression in a cell of interest. In some embodiments, the polynucleotide is codon optimized for expression in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell. In some embodiments, the cell is a mammalian cell, preferably a human cell.

In some embodiments, the polynucleotide is an mRNA and further comprises a 5′ cap sequence and/or a poly-A tail sequence. In some embodiments of the present invention, the mRNA utilized may be modified to enhance its functional properties and stability. Specifically, in some embodiments, the modification process involves the substitution of uridine (represented by the letter “U”) with N1-Methylpseudouridine or pseudouridine. This substitution is designed to improve the mRNA's resistance to degradation by ribonucleases, potentially increasing its half-life and translational efficiency within the cell. The incorporation of N1-Methylpseudouridine or pseudouridine into the mRNA structure can also positively influence the immune response profile, as these modifications have been shown to reduce the immunogenicity of mRNA molecules when compared to their unmodified counterparts. This is particularly crucial for the development of mRNA-based therapeutics and vaccines, where minimizing adverse immune reactions is paramount.

In some embodiments, the polynucleotide in this invention is codon-optimized for expression in a eukaryotic cell; optionally, the eukaryotic cell is selected from the group consisting of: a plant cell, a fungal cell, a single-cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and/or a human cell.

In some embodiments, the polynucleotide has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the nucleotide sequence of any one of SEQ ID NOs: 161-231, 241-311, 851-852, and 861-863.

This invention also provides an engineered, non-naturally occurring CRISPR-Cas system comprising: a) the Type II Cas protein described herein or the polynucleotide encoding the Cas protein thereof, b) at least one engineered guide RNA or at least one engineered nucleic acid encoding the guide RNA thereof, wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex.

In some embodiments, the guide RNA is a dual guide RNA. In some embodiments, the guide RNA is a single guide RNA. In such embodiments, the guide RNA further comprises a linker sequence connecting the tracrRNA sequence and the DR sequence. In some typical embodiments, the linker comprises a short sequence of GAAA. In some embodiments, the linker serves as an artificial loop. In some embodiments, the sgRNA comprises, in an arrangement: a) a spacer sequence, which is capable of hybridizing to a sequence of the target nucleic acid to be manipulated; b) a DR sequence; c) a linker sequence and d) tracrRNA sequence. The tandem arrangement of the spacer sequence, the DR sequence; the linker sequence and tracrRNA sequence is in a 5′ to 3′ orientation, or in a 3′ to 5′ orientation. In some embodiments, the sgRNA scaffold comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOs: 431-562.

In some embodiments, the sgRNA comprises a spacer sequence (such as any one of the sequences of SEQ ID NOs: 571-835, 885, and 901; and a scaffold sequence, and the spacer sequence is located at the 5′ end of the scaffold sequence (such as the sequences of SEQ ID NO: 903). In some embodiments, the sgRNA comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

In some embodiments, the spacer sequence hybridizes to one or more nucleic acid in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the system further comprising a donor template nucleic acid.

In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome). In some embodiments, the donor template nucleic acid is a DNA or an RNA or a DNA-RNA hybrid.

This invention also provides an engineered vector comprising the polynucleotide described in this disclosure.

In some embodiments, the vector is an expression vector. In some embodiments, the vector is an inducible, conditional, or constitutive expression vector. In some embodiments, the polynucleotide encoding the Cas protein and the polynucleotides encoding the guide RNA are on a same vector or on different vectors.

This invention also provides a vector system comprising one or more polynucleotides described in this disclosure and one or more polynucleotides encoding a guide RNA; wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex. In some embodiments, the polynucleotide encoding the Cas protein and the polynucleotides encoding the guide RNA are on a same vector or on different vectors.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

This invention also provides an engineered, non-naturally occurring cell comprising: the Cas protein described in this disclosure, the polynucleotide described in this disclosure, the CRISPR-Cas system described in this disclosure, the vector described in this disclosure, or the vector system described in this disclosure.

This invention also provides a cell modified by utilizing the Cas protein described in this disclosure, the polynucleotide described in this disclosure, the CRISPR-Cas system described in this disclosure, the vector described in this disclosure, or the vector system described in this disclosure.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In one embodiment, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

In some embodiments, the disclosure provides an isolated eukaryotic cell comprising a modified target locus of interest, wherein the target locus of interest has been modified according to a method or using of a composition or using of a system described in this invention.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In one embodiment, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

This invention also provides a kit comprising: the Cas protein described in this disclosure, the polynucleotide described in this disclosure, the CRISPR-Cas system described in this disclosure, the vector described in this disclosure, the vector system described in this disclosure, or the cell described in this disclosure.

The kits described in this disclosure may encompass one or more containers with components essential for performing the methods in this disclosure, and may optionally contain instructions for use. Any of the kits delineated may additionally comprise ancillary components required for the execution of the editing methods. Each component within the kits, where applicable, may be provided in a liquid form (e.g., dissolved in solution) or in a solid form (e.g., lyophilized powder). In specific embodiments, some components may be reconstituted or otherwise processed (e.g., to an active state) upon the addition of a suitable solvent or other substance (such as water or buffer), which may or may not be furnished with the kit. In some embodiments, the kit may further comprise other suitable excipients such as buffers or reagents for facilitating the application of the kit. Preferably, the kit may be applied in various applications such as medical applications including therapies and diagnosis, researches and the like. Accordingly, the type II Cas nuclease and the kit of the present invention may be used in the preparation of a medicament for treatment and/or in the preparation of an agent for research study.

The Cas protein, CRISPR-Cas system, polynucleotide described herein, can be delivered by various delivery systems such as vectors, e.g., plasmids, viral delivery vectors, such as adeno-associated viruses (AAV), lentiviruses, adenoviruses, and other viral vectors, or methods, such as nucleofection or electroporation of ribonucleoprotein complexes consisting of Type V-I effectors and their cognate RNA guide or guides. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and Φ174.

This invention also provides a pharmaceutical composition comprising: the Cas protein described in this disclosure, the polynucleotide described in this disclosure, the CRISPR-Cas system described in this disclosure, the vector described in this disclosure, the vector system described in this disclosure or the cell described in this disclosure.

As presented in this disclosure, the term “pharmaceutical composition” refers to a formulation intended for pharmaceutical use. In certain embodiments, the pharmaceutical composition further comprises a pharmaceutically acceptable excipient. In some embodiments, the pharmaceutical composition may include additional therapeutic agents. In some embodiments, the pharmaceutical composition is prepared following standard procedures for administration via intravenous, intramuscular, intradermal, intra-articular, intralesional, intraperitoneal, intracardiac, intrathecal, intracerebroventrical, epidural, topical, subconjunctival, intrastromal, peribulbar, intravitreal, posterior juxlascleral, transscleral, suprachoroidal, retrobulbar, subretinal, sub-tenon, nasal inhalational, pressurized inhalation, oral, subcutaneous or local routes to a subject, such as a human patient. For instance, compositions for injection may be provided as sterile isotonic aqueous solutions. If necessary, the pharmaceutical composition may also contain solubilizing agents and local anesthetics, such as lidocaine, to minimize injection site discomfort. Typically, components are supplied either separately or in admixture as a unit dose, e.g., as a lyophilized powder or a concentrated solution devoid of water, in a hermetically sealed container that indicates the quantity of the active agent(s). Where the pharmaceutical composition is intended for infusion, it may be combined with an infusion bottle containing sterile pharmaceutical-grade water or saline. Where the pharmaceutical composition is for injection, sterile water for injection or saline may be included to allow for component mixing prior to administration. Additionally, wetting agents, coloring agents, release agents, coating agents, sweetening agents, flavoring agents, perfuming agents, preservatives, and antioxidants may also be incorporated into the formulation as needed.

In some embodiments, the pharmaceutical composition further comprises a delivery system selected from: AAV (adeno-associated viruses), Adenoviruses, retroviruses, HSV (herpes simplex virus), Gammaretrovirus, LV (lentivirus), eCIS (extracellular Contractile Injection System), eVLPs (Engineered virus-like particles), VLPs (virus-like particles), liposomes, plasmid, LNPs (lipid nanoparticles), exosomes, microvesicles, nucleic acid nanoassemblies, a gene gun, and/or an implantable device.

In certain embodiments, the delivery is via adeno-associated viruses (AAV), e.g., AAV2, AAV8, or AAV9, which can be administered in a single dose containing at least 1×10⁵ particles (also referred to as particle units, pu) of adenoviruses or adeno-associated viruses.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AAV2, AAV5, AAV6, AAV8, AAV8.2. AAV9, AAV rh1O, modified AAV vectors (e.g., modified AAV2, modified AAV3, modified AAV6) and pseudotyped AAV (e.g., AAV2/8, AAV2/5 and AAV2/6).

In some embodiments, the delivery is via plasmids. The dosage can be a sufficient number of plasmids to elicit a response. In some embodiments, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR-Cas system, but one or more of these may instead be encoded on different vectors. The frequency of administration is within the ambit of the medical or veterinary practitioner (e.g., physician, veterinarian), or a person skilled in the art.

This invention also provides the use of the Cas protein, polynucleotide, CRISPR-Cas system, vector, vector system, cell, kit, or pharmaceutical composition described in this disclosure for the treatment, prevention, diagnosis, or detection of a disease.

This invention also provides a method of modifying or targeting a target DNA locus, wherein the method comprising delivering to said locus: the Cas protein described in this disclosure; the polynucleotide described in this disclosure; the CRISPR-Cas system described in this disclosure; the vector described in this disclosure, the vector system described in this disclosure the kit described in this disclosure or the pharmaceutical composition described in this disclosure.

In some embodiments, the disclosure also provides a method of targeting and cleaving a target DNA, the method comprising: contacting the target DNA with the Cas protein described in this disclosure; the polynucleotide described in this disclosure; the CRISPR-Cas system described in this disclosure; the vector described in this disclosure, the vector system described in this disclosure the kit described in this disclosure or the pharmaceutical composition described in this disclosure.

In some embodiments, said modifying or targeting a target locus comprises inducing a DNA strand break. In some embodiments, said modifying or targeting a target locus comprises inducing a DNA double strand break or a DNA single strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest.

In some embodiments, cleaving the target DNA or target sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites. In some embodiments, the target DNA is a double stranded DNA or a single stranded DNA, or DNA-RNA hybrids.

In some embodiments, said modifying or targeting a target locus comprises inducing a DNA strand break, altering gene expression of one or more genes, or epigenetic modification of said target DNA locus; optionally, the DNA strand break comprise a DNA double strand break or a DNA single strand break.

In some embodiments, the method is performed in vitro or in vivo.

This invention also provides an isolated eukaryotic cell comprising a modified target locus of interest, wherein the target locus of interest has been modified according to the method described in this disclosure, or by using the system described in this disclosure, or by using the Cas protein described in this disclosure; or by using the polynucleotide described in this disclosure; or by using the CRISPR-Cas system described in this disclosure; or by using the vector described in this disclosure, or by using the vector system described in this disclosure the, or by using the kit described in this disclosure, or by using the pharmaceutical composition described in this disclosure.

This invention also provides a system for detecting the presence of a nucleic acid target sequence in an in vitro sample, comprising: a) a Cas protein described in this disclosure; b) at least one guide polynucleotide comprising a guide sequence capable of binding the target sequence, and designed to form a complex with the Cas protein; and c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequence.

This invention also provides a method for detecting target nucleic acids in samples comprising: contacting one or more samples with a) a Cas protein described in this disclosure; b) at least one guide polynucleotide comprising a guide sequence designed to have a degree of complementarity with the target sequence, and designed to form a complex with the Cas protein; and c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequences; and detecting a signal from cleavage of the non-target sequence, thereby detecting the one or more target sequences in the sample.

This invention also provides a guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA further comprises a Cas protein binding segment; and wherein the Cas protein binding segment comprises a tracrRNA sequence and a direct repeat (DR) sequence that hybridizes to form a double-stranded RNA (dsRNA) duplex. In some embodiments, the gRNA is a dual guide RNA. In some embodiments, the gRNA is a single guide RNA. In some embodiments, the gRNA is modified. In some embodiments, at least three nucleotides of the gRNA are modified. In some embodiment, the gRNA comprises a 5′ end modification comprising at least two phosphorothioate (PS) linkages within the first seven nucleotides at the 5′ end of the 5′ terminus. In some embodiments, the gRNA comprises a 3′ end modification comprising at least two phosphorothioate (PS) linkages within the first seven nucleotides at the 3′ end of the 3′ terminus. In some embodiments, the gRNA has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the nucleotide sequence of any one of SEQ ID NO: 903.

This invention also provides polynucleotide encoding the gRNA described herein; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NO: 903.

This invention also provides an engineered, non-naturally occurring CRISPR-Cas system comprising: a) a Cas protein or a polynucleotide encoding the Cas protein thereof, b) at least one guide RNA (gRNA) described in this disclosure or at least one engineered nucleic acid encoding the guide RNA thereof, wherein said gRNA further comprises a Cas protein binding segment that interacts with said Cas protein; and wherein the Cas protein binding segment comprises a tracrRNA sequence and a direct repeat (DR) sequence that hybridizes to form a double-stranded RNA (dsRNA) duplex; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence of SEQ ID NO: 903.

In some embodiments, the Cas protein comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NO: 12; or the Cas protein comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NO: 12, with the exception of the amino acid “M” at position 1 of the sequence.

In some embodiments, the Cas protein further comprises an effector domain (or functional domain). Such effector domains can have one or more types of enzymatic activities, including polymerase activity, ligase activity, reverse transcriptase activity, deaminase activity, replication activity, or proofreading activity. In some embodiment, the effector domains domain comprises a nuclease, a nickase, a deaminase, a reverse transcriptase, a recombinase, a methyltransferase, a methylase, an acetylase, an acetyltransferase, a transcriptional activator, a transcriptional repressor domain, a cryptochrome, a light inducible/controllable domain, or a chemically inducible/controllable domain.

In some embodiments, the Cas protein further comprises one or more of a nuclear localization signal sequence, a nuclear export signal sequence, a cell penetrating peptide sequence, an affinity tag. the type II Cas protein comprises one or more nuclear localization signal(s) NLS(s). The NLS(s) can locate at the end or other portion of the peptide. The NLS(s) located each end or other portion of the Cas9 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas9 amino acid sequence comprising one NLS and the C-terminal end of the Cas9 amino acid sequence comprising one NLS. The amino acid sequence of NLS fused to the N-terminal end and/or the C-terminal end of the Cas9 amino acid sequence respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of C-terminal NLS is set forth in SEQ ID NO: 881 or 882. In some embodiments, the amino acid sequence of the C-terminal FLAG sequence is set forth in SEQ ID NO: 883. Other available sequences and different combinations can also be chosen for the NLSs sequences and FLAG sequence.

In some embodiments, the Cas protein comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 12.

In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and is capable of recognizing a protospacer adjacent motif (PAM) having a sequence of: NRHACT.

In some embodiments, the Cas protein is a nickase or dead Cas protein. The DNA cleavage domain of an active Cas protein in this invention include two subdomains, the HNH nuclease subdomain and the RuvC subdomain. Mutations within these subdomains can silence the nuclease activity of the Cas protein. In some embodiments, Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862, with the exception of the amino acid “M” at position 1 of the sequence.

In some embodiments, the mutation at residue D10 or H862 of SEQ ID NO: 12 is D10A or H862A. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 872-874.

This invention also provides an engineered vector comprising the polynucleotide encoding the gRNA described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

In some embodiments, said vector is optionally an inducible, conditional, or constitutive expression vector.

This invention also provides a vector system comprising one or more polynucleotide encoding the gRNA described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 871. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provides a pharmaceutical composition comprising the gRNA described in this disclosure; the polynucleotide encoding such gRNA, the CRISPR-Cas system comprising such gRNA or polynucleotide, the vector comprising such gRNA encoding sequence, the vector system such gRNA encoding sequence; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901, In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provide a method for treating, preventing, or diagnosing diseases associated with the RHO gene locus in a subject, comprising contacting the target cell with the gRNA described in this disclosure; b) contacting the target cell with the polynucleotide encoding such gRNA; c) contacting the target cell with the CRISPR-Cas system comprising such gRNA or polynucleotide; or d) contacting the target cell with the pharmaceutical described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provides a method of treating, preventing or diagnosing diseases associated with gene locus in a subject, comprising administering a) the gRNA described in this disclosure; b) administering the target cell with the polynucleotide encoding such gRNA; c) administering the target cell with the CRISPR-Cas system comprising such gRNA or polynucleotide; or d) administering the target cell with the pharmaceutical described in this disclosure; wherein the guide RNA (gRNA) comprising: i) a spacer sequence of SEQ ID NO: 901. ii) a spacer sequence having at least 15, 16, 17, 18, 19, or 20 contiguous nucleotides of the sequence of SEQ ID NO: 901; or iii) a spacer sequence with at least 99%, 98%, 97%, 96%, 95%, 94%, 93%, 92%, 91%, or 90% identity to the sequence of SEQ ID NO: 901. In some embodiments, the gRNA comprises: a sequence of SEQ ID NO: 903; or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provides a composition comprising: (i) a Cas protein, wherein:
a. the Cas protein comprises a sequence with at least 90% identity to SEQ ID NO: 12 or 92; and/or
b. the Cas protein comprises a sequence with at least 95%, 96%, 97%, 98%, 99%, 100% identity to SEQ ID NO: 12 or 92; and/or
   (ii) a sgRNA or a vector encoding a sgRNA, wherein the sgRNA comprises a sgRNA sequence of SEQ ID NO: 903.

This invention also provides a method of modifying the RHO gene locus, comprising delivering a composition to a cell, wherein the composition comprises:
a. a guide RNA comprising a guide sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of treating, preventing or diagnosing diseases associated with RHO in a subject, comprising administering a composition to a subject in need thereof, wherein the composition comprises:
a. a guide RNA comprising a guide sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of modifying the RHO gene locus, comprising delivering a composition to a cell, wherein the composition comprises:
a. a sgRNA comprising a sgRNA sequence of SEQ ID NO: 903;
b. a sgRNA comprising a sgRNA sequence with at least 90% identity to a sequence of SEQ ID NO: 903; or
c. a sgRNA comprising a sgRNA sequence with at least 99%, 98%, 97%, 96%, 95%, 94%, 93%, 92%, 91%, or 90% identity to a sequence of SEQ ID NO: 903.

This invention also provides a method of treating, preventing or diagnosing diseases associated with RHO in a subject, comprising administering a composition to a subject in need thereof, wherein the composition comprises:
a. a sgRNA comprising a sgRNA sequence of SEQ ID NO: 903;
b. a sgRNA comprising a sgRNA sequence with at least 90% identity to a sequence of SEQ ID NO: 903; or
c. a sgRNA comprising a sgRNA sequence with at least 99%, 98%, 97%, 96%, 95%, 94%, 93%, 92%, 91%, or 90% identity to a sequence of SEQ ID NO: 903.

This invention also provides a method of treating, preventing or diagnosing diseases associated with RHO in a subject, comprising administering a composition to a subject in need thereof, wherein the composition comprises:
a. a guide RNA comprising a spacer sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of modifying the RHO gene locus, comprising delivering a composition to a cell, wherein the composition comprises:
(i) a Cas protein, wherein:
   a. the Cas protein comprises a sequence with at least 90% identity to SEQ ID NO: 12 or 92; and/or
   b. the RNA-guided DNA binding agent comprises a sequence with at least 95%, 96%, 97%, 98%, 99%, 100% identity to SEQ ID NO: 12 or 92; and/or
(ii) a guide RNA or a vector encoding a guide RNA, wherein the guide RNA comprises a spacer sequence of SEQ ID NO: 901.

This invention also provides a method of treating, preventing or diagnosing diseases associated with RHO in a subject, comprising administering a composition to a subject in need thereof, wherein the composition comprises:
(i) an RNA-guided DNA binding agent, wherein:
   a. the RNA-guided DNA binding agent comprises a sequence with at least 90% identity to SEQ ID NO: 12 or 92; and/or
   b. the RNA-guided DNA binding agent comprises a sequence with at least 95%, 96%, 97%, 98%, 99%, 100% identity to SEQ ID NO: 12 or 92; and/or
(ii) a sgRNA or a vector encoding a sgRNA, wherein the sgRNA comprises a sequence of SEQ ID NO: 903.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 show the domain arrangement of GEBx type II Cas proteins;

FIGS. 2A-2E shows the PAM preference of Cas9 in HEK293 cell line;

FIG. 3 (FIG. 3) shows the indel levels of GEBx0305 across 16 targets with GGAAAA-PAM in HEK293T cell line (n=3);

FIG. 4 (FIG. 4) shows the indel levels of GEBx0308 across 19 targets with GGTACT-PAM in HEK293T cell line (n=3);

FIG. 5 (FIG. 5) shows the indel levels of GEBx0308 across 15 targets with CATACT-PAM in HEK293T cell line (n=3);

FIG. 6 (FIG. 6) shows the indel levels of GEBx0328 across 20 targets with NGGCCT-PAM in HEK293T cell line (n=3);

FIG. 7 shows the indel levels of GEBx0361 across 11 targets with GGTACC-PAM in HEK293T cell line (n=2).

FIG. 8 shows the indel levels of GEBx0361 across 8 targets with TGTACC-PAM in HEK293T cell line (n=2).

FIG. 9: A and B (FIG. 9: A and B) show indel levels vary depending on the length of the guide sequence of GEBx0305 (n=3);

FIG. 10: A and B (FIG. 10: A and B) show indel levels vary depending on the length of the guide sequence of GEBx0308 (n=3);

FIG. 11: A and B (FIG. 10: A and B) show indel levels vary depending on the length of the guide sequence of GEBx0328 (n=3);

FIG. 12: A and B (FIG. 12: A and B) show indel levels of GEBx0305 targeting endogenous genes with modified RNAs scaffold (n=3);

FIG. 13: A and B (FIG. 13: A and B) show indel levels of GEBx0308 targeting endogenous genes with modified RNAs scaffold (n=3);

FIG. 14: A and B (FIG. 14 A and B) show indel levels of GEBx0305 targeting endogenous genes under optimal conditions (n=3);

FIG. 15: A and B (FIG. 15: A and B) show indel levels of GEBx0308 targeting endogenous genes under optimal conditions (n=3);

FIG. 16: A and B (FIG. 16: A and B) show indel levels of GEBx0328 targeting endogenous genes with modified RNAs scaffold (n=3);

FIG. 17: A and B (FIG. 17: A and B) show indel levels of GEBx0328 targeting endogenous genes under optimal conditions (n=3);

FIG. 18 (FIG. 18) shows the indel levels of GEBx0305 targeting endogenous genes following transfection of HEK293T cell with lipoplex comprising a fixed amount (20 ng) of sgRNA and different ratios of mRNA. SpCas9 is used as positive control;

FIG. 19 (FIG. 19) shows the indel levels of GEBx0308 targeting endogenous genes following transfection of HEK293T cell with lipoplex comprising a fixed amount (20 ng) of sgRNA and different ratios of mRNA;

FIG. 20 (FIG. 20) shows the indel levels of GEBx0305 targeting endogenous genes following transfection of PHH cell with lipoplex comprising a fixed amount (20 ng) of sgRNA and different ratios of mRNA. SpCas9 is used as positive control;

FIG. 21 (FIG. 21) shows the indel levels of GEBx0308 targeting endogenous genes following transfection of PHH cell with lipoplex comprising a fixed amount (20 ng) of sgRNA and different ratios of mRNA;

FIG. 22 (FIG. 22) shows the summary of top Guide-seq insertion sites of GEBx0305 targeting (CFTR-NGGAAAA-T5) and site 2 (EMX1-NGGAAAA-T5); the sequences shown in the figure are SEQ ID NOs: 907-910;

FIG. 23 (FIG. 23) shows the summary of top Guide-seq insertion sites of GEBx0308 targeting site 1 (CD34-NGGTACT-T4) and site 2 (POLQ-NGGTACT-T1); the sequences shown in the figure are SEQ ID NOs: 911-914;

FIG. 24 (FIG. 24) shows the summary of top Guide-seq insertion sites of GEBx0328 targeting (CFTR-NGGCCT-T3) and site 2 (CFTR-NGGCCT-T5); the sequences shown in the figure are SEQ ID NOs: 915-919;

FIG. 25 (FIG. 25) shows the A to G conversion base editing frequency in HEK293T cells by GEBx0305-ABE at adenines for four sites;

FIG. 26 (FIG. 26) shows the A to G conversion base editing frequency in HEK293T cells by GEBx0308-ABE at adenines for five sites;

FIG. 27 (FIG. 27) shows the A to G conversion base editing frequency in HEK293T cells by GEBx0328-ABE at adenines for five sites.

FIG. 28 (FIG. 28) shows 4-Allele specific editing of GEBx0308 at RHO-P23H pathogenic site; the sequences shown in the figure are SEQ ID NOs: 920-923.

### DETAILED DESCRIPTION OF THE PREFERRED EMBODIMENT

The following examples further illustrate the present disclosure, but the present disclosure is not limited thereto.

It must be noted that as used herein and in the appended claims, the singular forms or the terms “a”, “an”, “the”, and “said” and similar terms used in the context of the present disclosure (especially in the context of the claims) are to be construed to cover both the singular and plural unless otherwise indicated herein or clearly contradicted by the context. In some embodiments, the above terms can be reasonably comprehended as “one” or “one or more”. Further, unless otherwise required by context, singular terms shall include pluralities and plural terms shall include the singular.

Unless otherwise indicated, it is intended that all singular/plural terms also encompass the active tense and past tense forms of a term, and it needs to be understood according to the context in the article.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and those terms such as “consisting essentially of” and “consists essentially of” have the meaning ascribed to them in U.S. Patent law. The term “a group consisting of” and the like refers to a specific set or collection of elements, components, or features. It may include one or more of the specified elements, components, or features. For example, a group consisting of: A, B, or C may refer to a set that includes any one or more of the specified elements A, B, or C. The claim encompasses the possibility of having any single element (A, B, or C) individually, any two elements combined (A and B, A and C, or B and C), or all three elements together (A, B, and C). This phrase defines the invention in terms of its variability within the specified options, allowing for different combinations of the listed elements while still maintaining the claimed scope.

When “t” or “T” appears in a sequence in this invention as a nucleotide of an RNA sequence, it should be understood as “u” or “U”.

The term “identity” in the context of two or more nucleic acids or polypeptide sequences refers to two or more sequences or subsequences that are the same or have a specified percentage of amino acid residues or nucleotides that are the same as measured using a BLAST or BLAST 2.0 or FASTA etc. sequence comparison algorithms with default parameters described below.

The term “exemplary” is used herein to mean serving as an example, instance, or illustration. Any aspect or design described herein as “exemplary” is not necessarily to be construed as preferred or advantageous over other aspects, embodiments, or designs.

As used herein, the term “optional” or “optionally” means that the subsequent described event, circumstance or substituent may or may not occur, and that the description includes instances where the event or circumstance occurs and instances where it does not.

The use of “or” or “/” is inclusive and means “and/or” unless stated otherwise; or it could be interpreted differently based on the context. The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “about”, “˜” as used herein when referring to a measurable value such as a parameter, an amount, a temporal duration, and the like, are meant to encompass variations of and from the specified value It is to be understood that the value to which the modifier “about” or refers is itself also specifically, and preferably, disclosed.

The term “exemplary” is used herein to mean serving as an example, instance, or illustration. Any aspect or design described herein as “exemplary” is not necessarily to be construed as preferred or advantageous over other aspects, embodiments, or designs.

The terms “subject”, “individual” and “patient” are used interchangeably herein to refer to a vertebrate, preferably a mammal, more preferably a human. Mammals include, but are not limited to mice, simians, humans, farm animals, sport animals, and pets. Tissues, cells and their progeny of a biological entity obtained in vivo or cultured in vitro are also encompassed.

“Variant” of sequence disclosed herein include sequence having one or more additions, deletions, stop positions, or substitutions, as compared to a sequence disclosed herein.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A polynucleotide encoding a protein includes all nucleotide sequences that are degenerate versions of each other and that code for the same amino acid sequence or amino acid sequences of substantially similar form and function.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In all aspects and embodiments, whether they include these terms or not, it will be understood that, preferably, may be optional and thus preferably included or not preferably included. Furthermore, the terms “non-naturally occurring” and “engineered” may be used interchangeably and so can therefore be used alone or in combination and one or other may replace mention of both together. In particular, “engineered” is preferred in place of “non-naturally occurring” or “non-naturally occurring and/or engineered” or “engineered, non-naturally occurring”.

As presented in this invention, the term “cleavage event” as used herein, refers to a DNA break in a target nucleic acid created by a type II Cas nuclease of a CRISPR system described herein. In some embodiments, the cleavage event is a double-stranded DNA break. In some embodiments, the cleavage event is a single-stranded DNA break.

As presented in this invention, the term “targeting” refers to the ability of a complex including a CRISPR-associated protein and an RNA guide, to preferentially or specifically bind to, e.g., hybridize to, a specific target nucleic acid compared to other nucleic acids that do not have the same or similar sequence as the target nucleic acid.

As presented in this disclosure, the term “GEBx” followed by a numerical suffix is utilized as a generic code to represent either nucleic acids or proteins. It is important to note that the use of identical codes for nucleic acids and proteins, or derivatives thereof, does not imply that the substances represented by these codes are identical. In other words, GEBx-0305 may refer to a specific nucleic acid sequence in one instance and a distinct protein in another. Some embodiments may illustrate a direct correspondence between the nucleic acid and protein denoted by the same or derived codes. Thus, the code “GEBx” serves as an indexing system to organize and reference the diverse biomolecules described in this disclosure, and the meaning of the code will be understood based on the context provided.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “in some embodiment(s)”, “in certain embodiment (s)”, “in some preferred embodiments”, “in some typical embodiment(s)”, “in typical embodiment(s)” or similar expressions means that a particular feature, structure or characteristic described in connection with the embodiment is included in at least one embodiment of the present disclosure. Furthermore, a particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any one of the claimed embodiments can be used in any combination.

The recitation of numerical ranges by endpoints includes all numbers and fractions subsumed within the respective ranges, as well as the recited endpoints.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “in a specific embodiment”, “in some embodiment”, “in certain embodiment” means that a particular feature, structure or characteristic described in connection with the embodiment is included in at least one embodiment of the present disclosure. Thus, appearances of the phrases “a specific embodiment”, “in one embodiment” or “in certain embodiment” in various places throughout this specification are not necessarily all referring to the same embodiment, but may. Furthermore, a particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any one of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

This invention provides an engineered, non-naturally occurring Type II CRISPR-associated (Cas) protein having a sequence identity of at least 70% to the amino acid sequence of any one of SEQ ID NOs: 1-71, or a variant thereof. In some embodiments, the Cas protein having a sequence identity of at least 75%, 80%, 85%, 90%, 92%, 95%, 98%, 99% or 100% to the amino acid sequence of any one of SEQ ID NOs: 1-71.

As presented in this invention, the “M” referred to herein stands for the starting amino acid Methionine, which is typically the initiating point for protein synthesis in many proteins. Naturally occurring Cas proteins often begin with Methionine as the first amino acid in their sequence. However, when scientists engineer these proteins, such as by fusing them with a Nuclear Localization Signal (NLS) or other domains, this initial amino acid “M” might be replaced or altered to introduce new functionalities or characteristics.

In this disclosure, particular attention has been given to the flexibility and functionality of the engineered Cas protein. Except for the intentional modification of the starting amino acid, Methionine (M), at position 1, designed Cas protein exhibits a significant sequence identity—ranging from 70% to 100%—when compared to the reference sequences. This strategic alteration not only aligns with our goal of tailoring the protein's properties but also ensures that the core or expected functionalities intrinsic to the Cas protein are preserved. By manipulating the initial amino acid without compromising the overall sequence similarity, some specific attributes are enhanced, such as improving cellular localization or introducing other advantageous features, while maintaining the fundamental characteristics that make Cas proteins indispensable tools in genomic manipulation.

This invention also provides an engineered, non-naturally occurring Type II CRISPR-associated (Cas) protein, wherein the Cas protein has a sequence identity of at least 70%, 75%, 80%, 85%, 90%, 92%, 95%, 98%, 99%, or 100% to the amino acid sequence of any one of SEQ ID NOs: 1-71, with the exception of the amino acid “M” at position 1 of the sequence.

As presented in this disclosure, the term “Cas protein”, “CRISPR-associated protein” or other similar terms refers to a class of CRISPR-associated proteins that are integral components of the CRISPR-Cas system. These proteins can possess intrinsic nuclease activity, enabling them to cleave double-stranded DNA or RNA molecules in a sequence-specific manner guided by a complementary RNA molecule, such as Cas9 and Cas12, which have been extensively utilized for genome editing applications. Additionally, some Cas proteins may be engineered to retain only one of the two active sites required for double-stranded cleavage, resulting in nickase activity, allowing them to introduce single-strand breaks in target nucleic acid sequences for controlled cleavage events. Furthermore, certain Cas proteins may be modified to lack any inherent nuclease activity, referred to as dead Cas or nuclease-inactive Cas proteins. Despite the absence of enzymatic function, these dead Cas proteins maintain their ability to bind specifically to target nucleic acid sequences and are often used in conjunction with other effector domains (or functional domains) for applications such as gene regulation, epigenome editing, and as components of advanced imaging systems. In some embodiments, the Cas protein may be used to reduce off-target effects. In some embodiments, the active Cas nuclease, nickase or dead Cas may also be part of a fusion protein containing another effector domain. The fusion proteins comprising such other effector domains (or functional domains) and such active Cas nuclease, nickase or dead Cas are also involved in the scope of the Cas protein. In some embodiments, the Cas protein may be a split form. In some embodiments, the Cas protein may also be an inducible Cas protein. In some embodiments, the type II Cas protein may be part of a self-inactivating system (SIN). In some embodiments, the type II Cas nuclease may also be part of a synergistic activator system (SAM) as defined herein elsewhere.

In some embodiments, the domain arrangement of Type II Cas protein the Cas protein contains a RuvC domain, BH (bridge helix) domain, REC domain, HNH domain, and/or a CTD (C-terminal domain). The RuvC domain is a critical catalytic site responsible for the cleavage of target DNA strands. It contains three split RuvC sub-domains, which are intricately folded to form the active site where DNA cleavage occurs. These sub-domains work in coordination to recognize and cleave the DNA at specific locations directed by the guide RNA. The BH domain, or bridge helix domain, serves as a structural link between the different domains of the Cas protein. It is characterized as an arginine-rich region comprising numbers of arginine amino acids. This abundance of arginine residues is crucial for interactions with the phosphate backbone of the target DNA strand. The arginine residues can form hydrogen bonds with the phosphate groups, aiding in the proper positioning and alignment of the target DNA for cleavage. The REC domain, or recognition lobe, is involved in the recognition of the target DNA sequence. It contributes to the specificity of the Cas protein by distinguishing between target and non-target sequences, ensuring that only the intended DNA segments are cleaved. The HNH domain is another catalytic site that works in conjunction with the RuvC domain to cleave the complementary strand of the target DNA. It is named after its characteristic histidine-asparagine-histidine sequence and is essential for the nuclease activity of the Cas protein. The CTD, or C-terminal domain, is often involved in interactions with other proteins or cellular structures, contributing to the localization and regulation of the Cas protein within the cell. It may also play a role in the stability and overall conformation of the Cas protein, ensuring that it remains functional and specific in its targeting. The complex domain arrangement allows the Type II Cas protein to carry out its precise and crucial function within the CRISPR system, making it an invaluable tool for genome editing and manipulation.

In some embodiments, the Cas protein further comprises an effector domain (or functional domain). Such effector domains can have one or more types of enzymatic activities, including polymerase activity, ligase activity, reverse transcriptase activity, deaminase activity, replication activity, or proofreading activity. In some embodiment, the effector domains domain comprises a nuclease, a nickase, a deaminase, a reverse transcriptase, a recombinase, a methyltransferase, a methylase, an acetylase, an acetyltransferase, a transcriptional activator, a transcriptional repressor domain, a cryptochrome, a light inducible/controllable domain, or a chemically inducible/controllable domain.

In some embodiments, the Cas protein further comprises one or more of a nuclear localization signal sequence, a nuclear export signal sequence, a cell penetrating peptide sequence, an affinity tag. the type II Cas protein comprises one or more nuclear localization signal(s) NLS(s). The NLS(s) can locate at the end or other portion of the peptide. The NLS(s) located each end or other portion of the Cas9 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas9 amino acid sequence comprising one NLS and the C-terminal end of the Cas9 amino acid sequence comprising one NLS. The amino acid sequence of NLS fused to the N-terminal end and/or the C-terminal end of the Cas9 amino acid sequence respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of C-terminal NLS is set forth in SEQ ID NO: 881 or 882. In some embodiments, the amino acid sequence of the C-terminal FLAG sequence is set forth in SEQ ID NO: 883. Other available sequences and different combinations can also be chosen for the NLSs sequences and FLAG sequence.

In some embodiments, the Cas protein, the Cas protein comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of any one of SEQ ID NOs: 1-71.

In some preferred embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, 12, 19, 21, 22, 24, 25, 27, 29, 30, 31, 36, 37, 38, 43, 44, 51, 56, 59, 60, 68). In some other preferred embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 9, 12, 19, 21, 22, 24, 25, 27, 29, 30, 31, 36, 37, 38, 43, 44, 51, 56, 59, 60, and 68, with the exception of the amino acid “M” at position 1 of the sequence.

One of the key elements of the CRISPR-Cas system associated modification process is the Protospacer Adjacent Motif (PAM), a short DNA sequence immediately adjacent to the target DNA sequence. The PAM sequence is essential for Cas protein binding and cleavage activity, ensuring that only the intended DNA sequences are targeted for editing. The ability of the Cas protein to recognize these specific PAM sequences is due to its unique structural features and binding interactions with the DNA. Each PAM sequence provides a distinct motif that the Cas protein can identify, ensuring accurate and efficient targeting. For example, the NRRANH sequence may offer a specific arrangement of nucleotides that the Cas protein can bind to with high affinity. These diverse range of PAM sequences expands the potential applications of the CRISPR-Cas system. It enables researchers and scientists to target a wider variety of DNA sequences for editing, increasing the versatility and effectiveness of this powerful gene-editing tool. Additionally, understanding these PAM sequences can aid in the development of more advanced Cas proteins with even greater targeting capabilities, further advancing the field of gene editing and its potential benefits for research, medicine, and biotechnology. The Cas protein, as disclosed in this invention, exhibits a unique capability to recognize a diverse range of PAM sequences. These sequences include NRRANH, NRHACT, NRAAR, NNNCCY, NNRYYYY, NGG, NNNNCAA, NRNACN, NNGR, NGGNR, NNNCCH, NRRAAG, NRHRAC, NRYART, NRHACC, NRAAR, NRNVHH, YMACAW, NAHAA, NRHAYY, or NGGHA. This specific recognition of these Cas proteins allows for greater flexibility in selecting target DNA sequences for editing.

As presented in this invention, in the PAM sequences provided, N: Represents any one of the four standard DNA nucleotides: Adenine (A), Thymine (T), Cytosine (C), or Guanine (G). This code facilitates the inclusion of any nucleotide at a given position without the need to specify it individually; R: Stands specifically for purine nucleotides, either Adenine (A) or Guanine (G). Purines are critical in DNA structure and function, and this code simplifies the incorporation of these larger nucleotides into PAM sequences; H: Denotes any nucleotide except Guanine (G). It can therefore represent Adenine (A), Thymine (T), or Cytosine (C). This code is useful for excluding Guanine (G) in specific positions where its larger size may impact structural or functional aspects of the sequence. Y: Indicates pyrimidine nucleotides, being either Thymine (T) or Cytosine (C). Pyrimidines are often found in specific regions of DNA and RNA, and this code aids in their inclusion without specifying the exact nucleotide. W: Represents weak bases, which can be either Adenine (A) or Thymine (T). This distinction is biochemically relevant as Adenine and Thymine have similar properties in certain contexts, such as hydrogen bonding. V: Reflects any nucleotide except Thymine (T), thus including Adenine (A), Cytosine (C), or Guanine (G). This code assists in situations where Thymine is not preferred or needed due to its unique chemical properties among the pyrimidines. M: Represents either Adenine (A) or Cytosine (C).

In some embodiments, the Cas protein, as disclosed in this invention, is capable of recognizing at least one protospacer adjacent motif (PAM) having or comprising a sequence of: NRRANH, NRHACT, NRAAR, NNNCCY, NNRYYYY, NGG, NNNNCAA, NRNACN, NNGR, NGGNR, NNNCCH, NRRAAG, NRHRAC, NRYART, NRHACC, NRAAR, NRNVHH, YMACAW, NAHAA, NRHAYY, or NGGHA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and is capable of recognizing a protospacer adjacent motif (PAM) having a sequence of: NRRANH. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and is capable of recognizing a protospacer adjacent motif (PAM) having a sequence of: NRHACT. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 19, and is capable of recognizing a protospacer adjacent motif (PAM) having a sequence of: NRAAR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 21, and is capable of recognizing a protospacer adjacent motif (PAM) having a sequence of: NNNCCY In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 22, and is capable of recognizing a protospacer adjacent motif (PAM) having a sequence of: NNRYYYY In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 24, and is capable of recognizing a protospacer adjacent motif (PAM) having a sequence of: NGG. In some embodiments, the Cas protein has a sequence identity of at least 70%, 71%, 72%, 73%, 74%, 75%, 76%, 77%, 78%, 79%, 80%, 8%1%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 25, and is capable of recognizing a protospacer adjacent motif (PAM) having a sequence of: NNNNCAA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 27, and is capable of recognizing a protospacer adjacent motif (PAM) having a sequence of: NRNACN. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 29, and is capable of recognizing a protospacer adjacent motif (PAM) having a sequence of: NNGR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 30, and is capable of recognizing a protospacer adjacent motif (PAM) having a sequence of: NGGNR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and is capable of recognizing a protospacer adjacent motif (PAM) having a sequence of: NNNCCH. In some embodiments, the Cas protein has a sequence identity of at least 770%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 36, and is capable of recognizing a protospacer adjacent motif (PAM) having a sequence of: NRRAAG. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 37, and is capable of recognizing a protospacer adjacent motif (PAM) having a sequence of: NRHRAC. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 38, and is capable of recognizing a protospacer adjacent motif (PAM) having a sequence of: NRYART. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 43, and is capable of recognizing a protospacer adjacent motif (PAM) having a sequence of: NRHACC. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 44, and is capable of recognizing a protospacer adjacent motif (PAM) having a sequence of: NRAAR. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 51, and is capable of recognizing a protospacer adjacent motif (PAM) having a sequence of: NRNVHH. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 56, and is capable of recognizing a protospacer adjacent motif (PAM) having a sequence of: YMACAW. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 59, and is capable of recognizing a protospacer adjacent motif (PAM) having a sequence of: NAHAA. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 60, and is capable of recognizing a protospacer adjacent motif (PAM) having a sequence of: NRHAYY. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 68, and is capable of recognizing a protospacer adjacent motif (PAM) having a sequence of: NGGHA.

As presented in this invention, the terms “recognized”, “recognizing”, or “recognition” in this context refers to the capability of the Cas protein to form a functional complex with a sgRNA at a DNA target site to which the sgRNA hybridizes (i.e. to which the spacer sequence of the sgRNA hybridizes) and being flanked by the PAM sequence, and wherein the Cas protein is capable of performing its natural function, i.e. DNA cleavage or DNA binding. In this context it is to be noted that such DNA cleavage precludes the type II Cas protein from being a catalytically inactive type II Cas nuclease. In the case of for instance an inactivated type II Cas nuclease (e.g. a dead type II Cas nuclease), a complex between the type II Cas nuclease, sgRNA and cognate target may nevertheless be formed if the required PAM sequence is present, but such does not result in DNA cleavage.

In some embodiments, the Cas protein is a nickase or dead Cas protein. The DNA cleavage domain of an active Cas protein in this invention include two subdomains, the HNH nuclease subdomain and the RuvC subdomain. Mutations within these subdomains can silence the nuclease activity of the Cas protein. In some embodiments, Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and includes a mutation at residue D11 or H859; or has a sequence identity of at least 70%, 71%, 72%, 73%, 74%, 75%, 76%, 77%, 78%, 79%, 80%, 81%, 82%8, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and includes a mutation at D12 or H903. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 9, and includes a mutation at residue D11 or H859, with the exception of the amino acid “M” at position 1 of the sequence; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862, with the exception of the amino acid “M” at position 1 of the sequence; or has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 31, and includes a mutation at D12 or H903, with the exception of the amino acid “M” at position 1 of the sequence. In some embodiments, the mutation at residue D11 or H859 of SEQ ID NO: 9 is D11A or H859A; the mutation at residue D10 or H862 of SEQ ID NO: 12 is D10A or H862A; or the mutation at residue D12 or H903 of SEQ ID NO: 31 is D12A or H903A. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 869-877.

This invention also provides an engineered, non-naturally occurring polynucleotide encoding the Type II CRISPR-associated (Cas) protein as disclosed in this invention.

As presented in this invention, the terms “polynucleotide” refers to a polymeric form of nucleotides of any length, either deoxyribonucleotides or ribonucleotides, or analogs thereof. Polynucleotides may have any three-dimensional structure, and may perform any function, known or unknown. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some embodiments, the polynucleotide encoding more than one portion of an expressed the type II Cas protein herein can be operably linked to each other and relevant regulatory sequences (such as promoters, enhancers, and termination regions). For example, there can be a functional linkage between a regulatory sequence and an exogenous nucleic acid sequence resulting in expression of the latter. For another embodiments, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame. In some embodiments, the promoter is a constitutive promoter, a tissue-specific promoter, or an inducible promoter. In some embodiments, the term further can include all introns and other DNA sequences spliced from the mRNA transcript, along with variants resulting from alternative splice sites. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

In some embodiments, the polynucleotide encoding the Cas protein is operably linked to a promoter and is presented in a vector; optionally, the vector is selected from the group consisting of: a retroviral vector, a lentiviral vector, a phage vector, an adenoviral vector, an adeno-associated virus vector, a herpes simplex virus vector and a plasmid vector.

In some embodiments, the polynucleotide is a ribonucleotide sequence or a deoxyribonucleotide sequence, or analogs thereof, optionally, the polynucleotide is codon-optimized for expression in a cell of interest. In some embodiments, the polynucleotide is codon optimized for expression in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell. In some embodiments, the cell is a mammalian cell, preferably a human cell.

In some embodiments, the polynucleotide is an mRNA and further comprises a 5′ cap sequence and/or a poly-A tail sequence. In some embodiments of the present invention, the mRNA utilized may be modified to enhance its functional properties and stability. Specifically, in some embodiments, the modification process involves the substitution of uridine (represented by the letter “U”) with N1-Methylpseudouridine or pseudouridine. This substitution is designed to improve the mRNA's resistance to degradation by ribonucleases, potentially increasing its half-life and translational efficiency within the cell. The incorporation of N1-Methylpseudouridine or pseudouridine into the mRNA structure can also positively influence the immune response profile, as these modifications have been shown to reduce the immunogenicity of mRNA molecules when compared to their unmodified counterparts. This is particularly crucial for the development of mRNA-based therapeutics and vaccines, where minimizing adverse immune reactions is paramount.

In some embodiments, the polynucleotide in this invention is codon-optimized for expression in a eukaryotic cell; optionally, the eukaryotic cell is selected from the group consisting of: a plant cell, a fungal cell, a single-cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and/or a human cell.

In some embodiments, the polynucleotide has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the nucleotide sequence of any one of SEQ ID NOs: 161-231, 241-311, 851-852, and 861-863.

This invention also provides an engineered, non-naturally occurring CRISPR-Cas system comprising: a) the Type II Cas protein described herein or the polynucleotide encoding the Cas protein thereof, b) at least one engineered guide RNA or at least one engineered nucleic acid encoding the guide RNA thereof, wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex.

As presented in this invention, the term “complementary” describes the ability of two nucleic acid strands to pair with each other through their bases. This complementarity can occur via perfect base-pairing, where each base along one strand forms a specific hydrogen-bonded pair with its complementary base on the opposite strand, following the standard Watson-Crick base pairing rules: adenine (A) pairs with thymine (T) or uracil (U), and cytosine (C) pairs with guanine (G). This perfect base-pairing allows for precise recognition and binding between the two strands. Additionally, complementarity can also involve imperfect base-pairing, which includes situations where mismatches, insertions, or deletions may lead to non-standard base pairing or reduced affinity between the strands. Despite these imperfections, the strands maintain enough complementarity to interact, albeit with potentially reduced specificity or stability in the duplex.

As presented in this invention, the direct repeat (DR) sequence originates from a short, repeated DNA sequence element within the CRISPR array. This sequence is typically interspersed between spacer sequences, which are derived from foreign genetic material such as phage or plasmid DNA. Upon transcription, the DR sequence is part of the pre-crRNA transcript and is processed into mature CRISPR RNA (crRNA). The DR sequence in the crRNA serves as a critical component that pairs with a complementary sequence in the trans-activating CRISPR RNA (tracrRNA) to form a double-stranded RNA (dsRNA) duplex. This duplex facilitates the binding of the Cas protein and is essential for the function of the guide RNA (gRNA) complex in the CRISPR/Cas system. In the context of the present invention, the DR sequence comprises the portion of the gRNA that hybridizes with the tracrRNA sequence to form the dsRNA duplex, thereby enabling the formation of the Cas protein binding segment. “hybridizes to form a double-stranded RNA (dsRNA) duplex”, or similar expressions, in this invention, refers to the process by which the direct repeat (DR) sequence of the CRISPR RNA (crRNA) pairs with a complementary sequence in the trans-activating CRISPR RNA (tracrRNA) to form a stable double-stranded RNA structure. This dsRNA duplex is a critical component of the guide RNA (gRNA) complex, which facilitates the binding of the Cas protein. Specifically, the DR sequence in the crRNA and the complementary sequence in the tracrRNA undergo base pairing, forming hydrogen bonds between their complementary bases, resulting in the formation of the dsRNA duplex. This duplex is essential for the function of the Cas protein binding segment, which comprises the tracrRNA sequence and the DR sequence that hybridizes to form the dsRNA duplex.

As presented in this invention, the term “target nucleic acid” refers to a specific nucleic acid substrate that contains a nucleic acid sequence complement to the entirety or a part of the spacer in an RNA guide. In some embodiments, the target nucleic acid comprises a gene or a sequence within a gene. In certain embodiments, the target nucleic acid comprises a noncoding region (e.g., a promoter). In a specific embodiment, the target nucleic acid is single-stranded. In a specific embodiment, the target nucleic acid is double-stranded. The term “target nucleic acid” or “target sequence” needs to be understood according to the context in the disclosure.

In some embodiments, the guide RNA is a dual guide RNA. In some embodiments, the guide RNA is a single guide RNA. In such embodiments, the guide RNA further comprises a linker sequence connecting the tracrRNA sequence and the DR sequence. In some typical embodiments, the linker comprises a short sequence of GAAA. In some embodiments, the linker serves as an artificial loop. In some embodiments, the sgRNA comprises, in an arrangement: a) a spacer sequence, which is capable of hybridizing to a sequence of the target nucleic acid to be manipulated; b) a DR sequence; c) a linker sequence and d) tracrRNA sequence. The tandem arrangement of the spacer sequence, the DR sequence; the linker sequence and tracrRNA sequence is in a 5′ to 3′ orientation, or in a 3′ to 5′ orientation. In some embodiments, the sgRNA scaffold comprises a sequence having at least 770%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOs: 431-562.

In some embodiments, the sgRNA comprises a spacer sequence (such as any one of the sequences of SEQ ID NOs: 571-835, 885, and 901) and a scaffold sequence, and the spacer sequence is located at the 5′ end of the scaffold sequence (such as the sequences of SEQ ID NO: 903). In some embodiments, the sgRNA comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

In some embodiments, the spacer sequence hybridizes to one or more nucleic acid in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the system further comprising a donor template nucleic acid.

As presented in this disclosure, the term “donor template nucleic acid” as used herein refers to a nucleic acid molecule that can be used by one or more cellular proteins to alter the structure of a target nucleic acid after a Cas protein described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome). In some embodiments, the donor template nucleic acid is a DNA or an RNA or a DNA-RNA hybrid.

This invention also provides an engineered vector comprising the polynucleotide described in this disclosure.

As presented in this disclosure, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In some embodiments, the vector is an expression vector. In some embodiments, the vector is an inducible, conditional, or constitutive expression vector. In some embodiments, the polynucleotide encoding the Cas protein and the polynucleotides encoding the guide RNA are on a same vector or on different vectors.

This invention also provides a vector system comprising one or more polynucleotides described in this disclosure and one or more polynucleotides encoding a guide RNA; wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex. In some embodiments, the polynucleotide encoding the Cas protein and the polynucleotides encoding the guide RNA are on a same vector or on different vectors.

This invention also provides an engineered, non-naturally occurring cell comprising: the Cas protein described in this disclosure, the polynucleotide described in this disclosure, the CRISPR-Cas system described in this disclosure, the vector described in this disclosure, or the vector system described in this disclosure.

This invention also provides a cell modified by utilizing the Cas protein described in this disclosure, the polynucleotide described in this disclosure, the CRISPR-Cas system described in this disclosure, the vector described in this disclosure, or the vector system described in this disclosure.

In some embodiments, the cell is a eukaryotic cell or a prokaryotic cell. In some embodiments, the eukaryotic cell is selected from the group consisting of a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In one embodiment, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

In some embodiments, the disclosure provides an isolated eukaryotic cell comprising a modified target locus of interest, wherein the target locus of interest has been modified according to a method or using of a composition or using of a system described in this invention.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In one embodiment, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adeno-associated viruses (AAV), e.g., AAV2, AAV8, or AAV9, which can be administered in a single dose containing at least 1×10⁵ particles (also referred to as particle units, pu) of adenoviruses or adeno-associated viruses. In some embodiments, the dose is at least about 1×10⁶ particles, at least about 1×10⁷ particles, at least about 1×10⁸ particles, or at least about 1×10⁹ particles of the adeno-associated viruses. Due to the limited genomic payload of recombinant AAV, the smaller size of the type II Cas nuclease described herein enables greater versatility in packaging the effector and RNA guides with the appropriate control sequences (e.g., promoters) required for efficient and cell-type specific expression.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AAV2, AAV5, AAV6, AAV8, AAV8.2. AAV9, AAV rh1O, modified AAV vectors (e.g., modified AAV2, modified AAV3, modified AAV6) and pseudotyped AAV (e.g., AAV2/8, AAV2/5 and AAV2/6).

In some embodiments, the delivery is via plasmids. The dosage can be a sufficient number of plasmids to elicit a response. In some embodiments, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR-Cas system, but one or more of these may instead be encoded on different vectors. The frequency of administration is within the ambit of the medical or veterinary practitioner (e.g., physician, veterinarian), or a person skilled in the art.

This invention also provides a kit comprising: the Cas protein described in this disclosure, the polynucleotide described in this disclosure, the CRISPR-Cas system described in this disclosure, the vector described in this disclosure, the vector system described in this disclosure, or the cell described in this disclosure.

The kits described in this disclosure may encompass one or more containers with components essential for performing the methods in this disclosure, and may optionally contain instructions for use. Any of the kits delineated may additionally comprise ancillary components required for the execution of the editing methods. Each component within the kits, where applicable, may be provided in a liquid form (e.g., dissolved in solution) or in a solid form (e.g., lyophilized powder). In specific embodiments, some components may be reconstituted or otherwise processed (e.g., to an active state) upon the addition of a suitable solvent or other substance (such as water or buffer), which may or may not be furnished with the kit. In some embodiments, the kit may further comprise other suitable excipients such as buffers or reagents for facilitating the application of the kit. Preferably, the kit may be applied in various applications such as medical applications including therapies and diagnosis, researches and the like. Accordingly, the type II Cas nuclease and the kit of the present invention may be used in the preparation of a medicament for treatment and/or in the preparation of an agent for research study.

The Cas protein, CRISPR-Cas system, polynucleotide described herein, can be delivered by various delivery systems such as vectors, e.g., plasmids, viral delivery vectors, such as adeno-associated viruses (AAV), lentiviruses, adenoviruses, and other viral vectors, or methods, such as nucleofection or electroporation of ribonucleoprotein complexes consisting of Type V-I effectors and their cognate RNA guide or guides. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and Φ174.

This invention also provides a pharmaceutical composition comprising: the Cas protein described in this disclosure, the polynucleotide described in this disclosure, the CRISPR-Cas system described in this disclosure, the vector described in this disclosure, the vector system described in this disclosure or the cell described in this disclosure.

As presented in this disclosure, the term “pharmaceutical composition” refers to a formulation intended for pharmaceutical use. In certain embodiments, the pharmaceutical composition further comprises a pharmaceutically acceptable excipient. In some embodiments, the pharmaceutical composition may include additional therapeutic agents. In some embodiments, the pharmaceutical composition is prepared following standard procedures for administration via intravenous, intramuscular, intradermal, intra-articular, intralesional, intraperitoneal, intracardiac, intrathecal, intracerebroventrical, epidural, topical, subconjunctival, intrastromal, peribulbar, intravitreal, posterior juxlascleral, transscleral, suprachoroidal, retrobulbar, subretinal, sub-tenon, nasal inhalational, pressurized inhalation, oral, subcutaneous or local routes to a subject, such as a human patient. For instance, compositions for injection may be provided as sterile isotonic aqueous solutions. If necessary, the pharmaceutical composition may also contain solubilizing agents and local anesthetics, such as lidocaine, to minimize injection site discomfort. Typically, components are supplied either separately or in admixture as a unit dose, e.g., as a lyophilized powder or a concentrated solution devoid of water, in a hermetically sealed container that indicates the quantity of the active agent(s). Where the pharmaceutical composition is intended for infusion, it may be combined with an infusion bottle containing sterile pharmaceutical-grade water or saline. Where the pharmaceutical composition is for injection, sterile water for injection or saline may be included to allow for component mixing prior to administration. Additionally, wetting agents, coloring agents, release agents, coating agents, sweetening agents, flavoring agents, perfuming agents, preservatives, and antioxidants may also be incorporated into the formulation as needed.

In some embodiments, the pharmaceutical composition further comprises a delivery system selected from: AAV (adeno-associated viruses), Adenoviruses, retroviruses, HSV (herpes simplex virus), Gammaretrovirus, LV (lentivirus), eCIS (extracellular Contractile Injection System), eVLPs (Engineered virus-like particles), VLPs (virus-like particles), liposomes, plasmid, LNPs (lipid nanoparticles), exosomes, microvesicles, nucleic acid nanoassemblies, a gene gun, and/or an implantable device.

This invention also provides the use of the Cas protein, polynucleotide, CRISPR-Cas system, vector, vector system, cell, kit, or pharmaceutical composition described in this disclosure for the treatment, prevention, diagnosis, or detection of a disease.

This invention also provides a method of modifying or targeting a target DNA locus, wherein the method comprising delivering to said locus: the Cas protein described in this disclosure; the polynucleotide described in this disclosure; the CRISPR-Cas system described in this disclosure; the vector described in this disclosure, the vector system described in this disclosure the kit described in this disclosure or the pharmaceutical composition described in this disclosure.

In some embodiments, the disclosure also provides a method of targeting and cleaving a target DNA, the method comprising: contacting the target DNA with the Cas protein described in this disclosure; the polynucleotide described in this disclosure; the CRISPR-Cas system described in this disclosure; the vector described in this disclosure, the vector system described in this disclosure the kit described in this disclosure or the pharmaceutical composition described in this disclosure.

In some embodiments, said modifying or targeting a target locus comprises inducing a DNA strand break. In some embodiments, said modifying or targeting a target locus comprises inducing a DNA double strand break or a DNA single strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest.

In some embodiments, cleaving the target DNA or target sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites. In some embodiments, the target DNA is a double stranded DNA or a single stranded DNA, or DNA-RNA hybrids.

In some embodiments, said modifying or targeting a target locus comprises inducing a DNA strand break, altering gene expression of one or more genes, or epigenetic modification of said target DNA locus; optionally, the DNA strand break comprise a DNA double strand break or a DNA single strand break.

In some embodiments, the method is performed in vitro or in vivo.

This invention also provides an isolated eukaryotic cell comprising a modified target locus of interest, wherein the target locus of interest has been modified according to the method described in this disclosure, or by using the system described in this disclosure, or by using the Cas protein described in this disclosure; or by using the polynucleotide described in this disclosure; or by using the CRISPR-Cas system described in this disclosure; or by using the vector described in this disclosure, or by using the vector system described in this disclosure the, or by using the kit described in this disclosure, or by using the pharmaceutical composition described in this disclosure.

This invention also provides a system for detecting the presence of a nucleic acid target sequence in an in vitro sample, comprising: a) a Cas protein described in this disclosure; b) at least one guide polynucleotide comprising a guide sequence capable of binding the target sequence, and designed to form a complex with the Cas protein; and c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequence.

This invention also provides a method for detecting target nucleic acids in samples comprising: contacting one or more samples with a) a Cas protein described in this disclosure; b) at least one guide polynucleotide comprising a guide sequence designed to have a degree of complementarity with the target sequence, and designed to form a complex with the Cas protein; and c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequences; and detecting a signal from cleavage of the non-target sequence, thereby detecting the one or more target sequences in the sample.

As present in this disclosure, a “sample” may contain whole cells and/or live cells and/or cell debris. The sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Samples include cell cultures, bodily fluids, cell cultures from bodily fluids. Bodily fluids may be obtained from a mammal organism, for example by puncture, or other collecting or sampling procedures.

This invention also provides a guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA further comprises a Cas protein binding segment; and wherein the Cas protein binding segment comprises a tracrRNA sequence and a direct repeat (DR) sequence that hybridizes to form a double-stranded RNA (dsRNA) duplex. In some embodiments, the gRNA is a dual guide RNA. In some embodiments, the gRNA is a single guide RNA. In some embodiments, the gRNA is modified. In some embodiments, at least three nucleotides of the gRNA are modified. In some embodiment, the gRNA comprises a 5′ end modification comprising at least two phosphorothioate (PS) linkages within the first seven nucleotides at the 5′ end of the 5′ terminus. In some embodiments, the gRNA comprises a 3′ end modification comprising at least two phosphorothioate (PS) linkages within the first seven nucleotides at the 3′ end of the 3′ terminus. In some embodiments, the gRNA has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the nucleotide sequence of SEQ ID NO: 903.

This invention also provides polynucleotide encoding the gRNA described herein; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

An engineered, non-naturally occurring CRISPR-Cas system comprising: a) a Cas protein or a polynucleotide encoding the Cas protein thereof, b) at least one guide RNA (gRNA) described in this disclosure or at least one engineered nucleic acid encoding the guide RNA thereof, wherein said gRNA further comprises a Cas protein binding segment that interacts with said Cas protein; and wherein the Cas protein binding segment comprises a tracrRNA sequence and a direct repeat (DR) sequence that hybridizes to form a double-stranded RNA (dsRNA) duplex; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

In some embodiments, the Cas protein comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 12; or the Cas protein comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 12, with the exception of the amino acid “M” at position 1 of the sequence.

In some embodiments, the Cas protein further comprises an effector domain (or functional domain). Such effector domains can have one or more types of enzymatic activities, including polymerase activity, ligase activity, reverse transcriptase activity, deaminase activity, replication activity, or proofreading activity. In some embodiment, the effector domains domain comprises a nuclease, a nickase, a deaminase, a reverse transcriptase, a recombinase, a methyltransferase, a methylase, an acetylase, an acetyltransferase, a transcriptional activator, a transcriptional repressor domain, a cryptochrome, a light inducible/controllable domain, or a chemically inducible/controllable domain.

In some embodiments, the Cas protein further comprises one or more of a nuclear localization signal sequence, a nuclear export signal sequence, a cell penetrating peptide sequence, an affinity tag. the type II Cas protein comprises one or more nuclear localization signal(s) NLS(s). The NLS(s) can locate at the end or other portion of the peptide. The NLS(s) located each end or other portion of the Cas9 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas9 amino acid sequence comprising one NLS and the C-terminal end of the Cas9 amino acid sequence comprising one NLS. The amino acid sequence of NLS fused to the N-terminal end and/or the C-terminal end of the Cas9 amino acid sequence respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of C-terminal NLS is set forth in SEQ ID NO: 881 or 882. In some embodiments, the amino acid sequence of the C-terminal FLAG sequence is set forth in SEQ ID NO: 883. Other available sequences and different combinations can also be chosen for the NLSs sequences and FLAG sequence.

In some embodiments, the Cas protein, the Cas protein comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 12.

In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and is capable of recognizing a protospacer adjacent motif (PAM) having a sequence of: NRHACT.

In some embodiments, the Cas protein is a nickase or dead Cas protein. The DNA cleavage domain of an active Cas protein in this invention include two subdomains, the HNH nuclease subdomain and the RuvC subdomain. Mutations within these subdomains can silence the nuclease activity of the Cas protein. In some embodiments, Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of SEQ ID NO: 12, and includes a mutation at residue D10 or H862, with the exception of the amino acid “M” at position 1 of the sequence.

In some embodiments, the mutation at residue D10 or H862 of SEQ ID NO: 12 is D10A or H862A. In some embodiments, the Cas protein has a sequence identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to the amino acid sequence of any one of SEQ ID NOs: 872-874.

This invention also provides an engineered vector comprising the polynucleotide encoding the gRNA described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

In some embodiments, said vector is optionally an inducible, conditional, or constitutive expression vector.

This invention also provides a vector system comprising one or more polynucleotide encoding the gRNA described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 871. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provides a pharmaceutical composition comprising the gRNA described in this disclosure; the polynucleotide encoding such gRNA, the CRISPR-Cas system comprising such gRNA or polynucleotide, the vector comprising such gRNA encoding sequence, the vector system such gRNA encoding sequence; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901, In some embodiments, the gRNA comprises: a) a sequence of SEQ ID NO: 903; or b) a sequence having at least 770%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provide a method for treating, preventing, or diagnosing diseases associated with the RHO gene locus in a subject, comprising contacting the target cell with the gRNA described in this disclosure; b) contacting the target cell with the polynucleotide encoding such gRNA; c) contacting the target cell with the CRISPR-Cas system comprising such gRNA or polynucleotide; or d) contacting the target cell with the pharmaceutical described in this disclosure; wherein the guide RNA (gRNA) comprising: a) a spacer sequence of SEQ ID NO: 901; b) a spacer sequence having at least 15, 16, 17, 18, 19, or 20 contiguous nucleotides of the sequence of SEQ ID NO: 901; c) a spacer sequence with at least 99%, 98%, 97%, 96%, 95%, 94%, 93%, 92%, 91%, or 90% identity to the sequence of SEQ ID NO: 901. In some embodiments, the gRNA comprises: a) a sequence of SEQ ID NO: 903; or b) a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

As presented in this disclosure, diseases associated with the “RHO gene locus” refer to a group of disorders characterized by mutations in the RHO gene. The RHO gene encodes the rhodopsin protein, which is essential for photoreceptor function in the retina. Mutations in this gene can lead to various retinal degenerative diseases, primarily affecting rod photoreceptors, and can result in vision loss or blindness. These disorders typically manifest as inherited retinal dystrophies, such as retinitis pigmentosa, which is characterized by progressive vision loss due to the degeneration of photoreceptor cells, particularly rods. Patients with mutations in the RHO gene may experience night blindness and a gradual loss of peripheral vision, and these conditions are often inherited in an autosomal dominant manner.

This invention also provides a method of treating, preventing or diagnosing diseases associated with gene locus in a subject, comprising administering a) the gRNA described in this disclosure; b) administering the target cell with the polynucleotide encoding such gRNA; c) administering the target cell with the CRISPR-Cas system comprising such gRNA or polynucleotide; or d) administering the target cell with the pharmaceutical described in this disclosure; wherein the guide RNA (gRNA) comprising: i) a spacer sequence of SEQ ID NO: 901. ii) a spacer sequence having at least 15, 16, 17, 18, 19, or 20 contiguous nucleotides of the sequence of SEQ ID NO: 901; or iii) a spacer sequence with at least 99%, 98%, 97%, 96%, 95%, 94%, 93%, 92%, 91%, or 90% identity to the sequence of SEQ ID NO: 901. In some embodiments, the gRNA comprises: a sequence of SEQ ID NO: 903; or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 903.

This invention also provides a composition comprising: (i) an RNA-guided DNA binding agent, wherein:
a. the RNA-guided DNA binding agent comprises a sequence with at least 90% identity to SEQ ID NO: 12 or 92; and/or
b. the RNA-guided DNA binding agent comprises a sequence with at least 95%, 96%, 97%, 98%, 99%, 100% identity to SEQ ID NO: 12 or 92; and/or
   (ii) a sgRNA or a vector encoding a sgRNA, wherein the sgRNA comprises a sgRNA sequence of SEQ ID NO: 903.

This invention also provides a method of modifying the RHO gene locus, comprising delivering a composition to a cell, wherein the composition comprises:
a. a guide RNA comprising a guide sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of treating, preventing or diagnosing diseases associated with RHO in a subject, comprising administering a composition to a subject in need thereof, wherein the composition comprises:
a. a guide RNA comprising a guide sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of modifying the RHO gene locus, comprising delivering a composition to a cell, wherein the composition comprises:
a. a sgRNA comprising a sgRNA sequence of SEQ ID NO: 903;
b. a sgRNA comprising a sgRNA sequence with at least 90% identity to a sequence of SEQ ID NO: 903; or
c. a sgRNA comprising a sgRNA sequence with at least 99%, 98%, 97%, 96%, 95%, 94%, 93%, 92%, 91%, or 90% identity to a sequence of SEQ ID NO: 903.

This invention also provides a method of treating, preventing or diagnosing diseases associated with RHO in a subject, comprising administering a composition to a subject in need thereof, wherein the composition comprises:
a. a sgRNA comprising a sgRNA sequence of SEQ ID NO: 903;
b. a sgRNA comprising a sgRNA sequence with at least 90% identity to a sequence of SEQ ID NO: 903; or
c. a sgRNA comprising a sgRNA sequence with at least 99%, 98%, 97%, 96%, 95%, 94%, 93%, 92%, 91%, or 90% identity to a sequence of SEQ ID NO: 903.

This invention also provides a method of treating, preventing or diagnosing diseases associated with RHO in a subject, comprising administering a composition to a subject in need thereof, wherein the composition comprises:
a. a guide RNA comprising a spacer sequence of SEQ ID NO: 901;
b. a guide RNA comprising at least 17, 18, 19 or 20 contiguous nucleotides of a sequence of SEQ ID NO: 901; or
c. a guide RNA comprising a guide sequence that with at least 99%, 98%, 97%, 96%, 95%, 94%, 93%, 92%, 91%, or 90% identity to a sequence of SEQ ID NO: 901.

This invention also provides a method of modifying the RHO gene locus, comprising delivering a composition to a cell, wherein the composition comprises:
(i) a Cas protein, wherein:
   a. the Cas protein comprises a sequence with at least 90% identity to SEQ ID NO: 12 or 92; and/or
   b. the RNA-guided DNA binding agent comprises a sequence with at least 95%, 96%, 97%, 98%, 99%, 100% identity to SEQ ID NO: 12 or 92; and/or
(ii) a guide RNA or a vector encoding a guide RNA, wherein the guide RNA comprises a spacer sequence of SEQ ID NO: 901.

This invention also provides a method of treating, preventing or diagnosing diseases associated with RHO in a subject, comprising administering a composition to a subject in need thereof, wherein the composition comprises:
(i) an RNA-guided DNA binding agent, wherein:
   a. the RNA-guided DNA binding agent comprises a sequence with at least 90% identity to SEQ ID NO: 12 or 92; and/or
   b. the RNA-guided DNA binding agent comprises a sequence with at least 95%, 96%, 97%, 98%, 99%, 100% identity to SEQ ID NO: 12 or 92; and/or
(ii) a sgRNA or a vector encoding a sgRNA, wherein the sgRNA comprises a sequence of SEQ ID NO: 903.

TABLE 1
The exemplary amino acid sequences involved in this disclosure

| Name | SEQ ID NOs: | Name | SEQ ID NOs: | Name | SEQ ID NOs: |
|---|---|---|---|---|---|
| GEBx0293 | 1 | GEBx0321 | 25 | GEBx0347 | 49 |
| GEBx0294 | 2 | GEBx0323 | 26 | GEBx0348 | 50 |
| GEBx0295 | 3 | GEBx0324 | 27 | GEBx0351 | 51 |
| GEBx0296 | 4 | GEBx0325 | 28 | GEBx0352 | 52 |
| GEBx0299 | 5 | GEBx0326 | 29 | GEBx0353 | 53 |
| GEBx0300 | 6 | GEBx0327 | 30 | GEBx0354 | 54 |
| GEBx0301 | 7 | GEBx0328 | 31 | GEBx0356 | 55 |
| GEBx0303 | 8 | GEBx0329 | 32 | GEBx0357 | 56 |
| GEBx0305 | 9 | GEBx0330 | 33 | GEBx0358 | 57 |
| GEBx0306 | 10 | GEBx0331 | 34 | GEBx0359 | 58 |
| GEBx0307 | 11 | GEBx0332 | 35 | GEBx0360 | 59 |
| GEBx0308 | 12 | GEBx0333 | 36 | GEBx0361 | 60 |
| GEBx0309 | 13 | GEBx0334 | 37 | GEBx0363 | 61 |
| GEBx0310 | 14 | GEBx0335 | 38 | GEBx0364 | 62 |
| GEBx0311 | 15 | GEBx0336 | 39 | GEBx0375 | 63 |
| GEBx0312 | 16 | GEBx0337 | 40 | GEBx0377 | 64 |
| GEBx0313 | 17 | GEBx0338 | 41 | GEBx0378 | 65 |
| GEBx0314 | 18 | GEBx0340 | 42 | GEBx0379 | 66 |
| GEBx0315 | 19 | GEBx0341 | 43 | GEBx0380 | 67 |
| GEBx0316 | 20 | GEBx0342 | 44 | GEBx0381 | 68 |
| GEBx0317 | 21 | GEBx0343 | 45 | GEBx0382 | 69 |
| GEBx0318 | 22 | GEBx0344 | 46 | GEBx0383 | 70 |
| GEBx0319 | 23 | GEBx0345 | 47 | GEBx0384 | 71 |
| GEBx0320 | 24 | GEBx0346 | 48 | | |
| GEBx0293 with | 81 | GEBx0321 with | 105 | GEBx0347 with | 129 |
| NLS and Flag | | NLS and Flag | | NLS and Flag | |
| GEBx0294 with | 82 | GEBx0323 with | 106 | GEBx0348 with | 130 |
| NLS and Flag | | NLS and Flag | | NLS and Flag | |
| GEBx0295 with | 83 | GEBx0324 with | 107 | GEBx0351 with | 131 |
| NLS and Flag | | NLS and Flag | | NLS and Flag | |
| GEBx0296 with | 84 | GEBx0325 with | 108 | GEBx0352 with | 132 |
| NLS and Flag | | NLS and Flag | | NLS and Flag | |
| GEBx0299 with | 85 | GEBx0326 with | 109 | GEBx0353 with | 133 |
| NLS and Flag | | NLS and Flag | | NLS and Flag | |
| GEBx0300 with | 86 | GEBx0327 with | 110 | GEBx0354 with | 134 |
| NLS and Flag | | NLS and Flag | | NLS and Flag | |
| GEBx0301 with | 87 | GEBx0328 with | 111 | GEBx0356 with | 135 |
| NLS and Flag | | NLS and Flag | | NLS and Flag | |
| GEBx0303 with | 88 | GEBx0329 with | 112 | GEBx0357 with | 136 |
| NLS and Flag | | NLS and Flag | | NLS and Flag | |
| GEBx0305 with | 89 | GEBx0330 with | 113 | GEBx0358 with | 137 |
| NLS and Flag | | NLS and Flag | | NLS and Flag | |
| GEBx0306 with | 90 | GEBx0331 with | 114 | GEBx0359 with | 138 |
| NLS and Flag | | NLS and Flag | | NLS and Flag | |
| GEBx0307 with | 91 | GEBx0332 with | 115 | GEBx0360 with | 139 |
| NLS and Flag | | NLS and Flag | | NLS and Flag | |
| GEBx0308 with | 92 | GEBx0333 with | 116 | GEBx0361 with | 140 |
| NLS and Flag | | NLS and Flag | | NLS and Flag | |
| GEBx0309 with | 93 | GEBx0334 with | 117 | GEBx0363 with | 141 |
| NLS and Flag | | NLS and Flag | | NLS and Flag | |
| GEBx0310 with | 94 | GEBx0335 with | 118 | GEBx0364 with | 142 |
| NLS and Flag | | NLS and Flag | | NLS and Flag | |
| GEBx0311 with | 95 | GEBx0336 with | 119 | GEBx0375 with | 143 |
| NLS and Flag | | NLS and Flag | | NLS and Flag | |
| GEBx0312 with | 96 | GEBx0337 with | 120 | GEBx0377 with | 144 |
| NLS and Flag | | NLS and Flag | | NLS and Flag | |
| GEBx0313 with | 97 | GEBx0338 with | 121 | GEBx0378 with | 145 |
| NLS and Flag | | NLS and Flag | | NLS and Flag | |
| GEBx0314 with | 98 | GEBx0340 with | 122 | GEBx0379 with | 146 |
| NLS and Flag | | NLS and Flag | | NLS and Flag | |
| GEBx0315 with | 99 | GEBx0341 with | 123 | GEBx0380 with | 147 |
| NLS and Flag | | NLS and Flag | | NLS and Flag | |
| GEBx0316 with | 100 | GEBx0342 with | 124 | GEBx0381 with | 148 |
| NLS and Flag | | NLS and Flag | | NLS and Flag | |
| GEBx0317 with | 101 | GEBx0343 with | 125 | GEBx0382 with | 149 |
| NLS and Flag | | NLS and Flag | | NLS and Flag | |
| GEBx0318 with | 102 | GEBx0344 with | 126 | GEBx0383 with | 150 |
| NLS and Flag | | NLS and Flag | | NLS and Flag | |
| GEBx0319 with | 103 | GEBx0345 with | 127 | GEBx0384 with | 151 |
| NLS and Flag | | NLS and Flag | | NLS and Flag | |
| GEBx0320 with | 104 | GEBx0346 with | 128 | | |
| NLS and Flag | | NLS and Flag | | | |

Representaive sequences above, NLS and Flag

| Name | SEQ ID NOs: | Sequences |
|---|---|---|
| GEBx0305 | 9 | MGQKDFYLGLDIGTDSVGYAVTDEQYNLLKFSGDAAWGVHLF |
| | | DEAELSQERRAFRVARRRLDRRKQRVMLLQELFAPAIRAVDAD |
| | | FYKRLQASDLFRDEAGDRFPIFNDVDYTDRDYYKAYPTIHHLIC |
| | | DLMADETAHDVRLVYLACAWLVSNRGHFLSNLDKENISALKN |
| | | FDKVYSEFEHFFTDNGYAYPWAEADRRTIGEILKKKTTVTAKN |
| | | KELTLAFTGGGMAPKEASEDFPFGQRAIIKLLAGGTAQLKELFD |
| | | QEAYAEFGAVSLNFDDDKLEEIAGNLGDDFALITALRQVFDWS |
| | | VLVEAIGDAASISEAKVETYRQHKADLAILKRIIKNYRPEKYDE |
| | | VFREIGKANYAAYAYHSNKKADAKFKKVDREAFSKFITGIVKD |
| | | ITPKAEDEEILADMRNRLDLRTFLPKQKNTDNRVIPHQLYQYEL |
| | | HEILKNAEGYLPFLTEKDESGLSVSDKIRSIFAFKIPYFVGPLNEK |
| | | SDHAWIVRKAGKIYPWNFEDIVDLDASEEGFIRRMTNQCTYLP |
| | | GEPVLPKDSLLYHKFTVLNEINNLSIHGERISVELKQQIYSDLFL |
| | | NKKKVTRKRLREYLIVNGVVEKDDTDAITGIDEEIKSNLLPQISF |
| | | RRLLDEGTLSEEDVEKIIERSTFSDDKTRLKKWLAREYPQLNSK |
| | | DVDYIRSLRLRDFGRLSQAFLEGIIAADRSTGEAFTIISALWNTQ |
| | | KNLMELLSDQFTFAETIERFREDYYREHPVSLSERMDEMYLSNS |
| | | VKRSVFRTLDIVKDVKKAFGAPKKIFVEVTRGGSPDQKGKRTK |
| | | SRSQQLRELYDKCRDEDLPLLKQQLEAMGDAADSKLQSEKLFL |
| | | YYLQLGKSAYSDKRIPLEALASKAYDIDHIYPQAFVKDDSIINN |
| | | KVLVLSEENGAKSNNYPIQGSIRHHMQGFWSYLQEVGLMSKE |
| | | KYKRLTRSTPFTEEERYGFINRQLTSTSQSTKAVATLLKEHLPET |
| | | EIVYCKARLVTDFRQAFDLFKSRQYNDLHHAVDAYLNIVTGNV |
| | | YNMRFTKNFNVHSQYSIKTETVFTRPVTYKSQTIWDGIDMLAK |
| | | VKSIANKNTAHITKYAFMKKGGFFDQLPVRAADGLIPRKKHLP |
| | | TQKYGGYNKPGVMFFIPVKYQLGKKSELFILPVELLCGKRFLA |
| | | DPEFAQAYAVKRLERILKKPVDQISFPLGMRPWKVNTVLALDG |
| | | FKYCITGSSNGGTRLILQPMMSFSSAPYWAFYLKKLEVFVEKN |
| | | ANNPQHVYDEAYDKVNCEDNLKLYDLYCQKLTSTIYSKRINSP |
| | | LDILLQGREKFSGLDIKQQAQALLNIHQVFGRIAGGCDLTLVGG |
| | | KKGSAATSASSAISNLGKRYQDVRVWDTSPSGLWSKRSPNLLD |
| | | LL |
| GEBx0308 | 12 | MKQKYYLGLDMGTSSLGWAVTNEKYSLLRKKGKDLWGVRLF |
| | | EEAKTAADRRSYRTARRRLQREKARIGYLRELFSEEINKIDAGF |
| | | FQRLEDSKYHKEDKREHQPFALFADKGYTDREYFEDYPTIFHL |
| | | RKALLDIENEGKTFDVRLVYLAILNMFKHRGHFLNANLDDKSG |
| | | GDLKEKIHILTSMANKLFDDISFKTVDIKDIEQILASKDYSNSTR |
| | | LEKLLELFELSKTKNKVETEIFKMICGLKGTISKIFANESMDEEA |
| | | QKLSFSFRDGNYDEKIVEMEEALSEESYEFIMIMKQVHDWAVL |
| | | SNIMMGEEYLSVARVKAYEKHKKDLNMLKALLKNYSIKEYNS |
| | | MFRIMKDNNYSSYIGSVISQETIRRKGKYTTDDFYKNVKSIVLK |
| | | CEECTEKEYILSEIEKKTFLPKQNTTSNSVIPNQVHKKELKRILE |
| | | NAAKYLEFLNDKDDTGLTVSEKIVRLFEFQIPYYVGPLSNGVCD |
| | | GKNVWSVRREKGAVLPWNFEEKIDVKQSSENFITKLVNHCTYL |
| | | NGETVLPKNSLLYEKFMVLNELNNLKINGVKIGVELKQSIYND |
| | | VFKKGKKVTAKALIQYLKNIGEIDATEETVVSGIDGDFTNRLAN |
| | | YKKFIEIFKVPTLTYEQEQIAEDIIFYSTVYGESKKFLEERIREKY |
| | | NEELSENQIKRILGLKFKDWGRLSKELLLLQGIDKQTGEISSIISK |
| | | MWSDNYNLMELIATDKFSYGYNIEQKTSKIEKVLSDIEYSDLDE |
| | | IYISAPVKRMVWQTILVLKELEKVMGCEPEKIFVEMARDANAP |
| | | KKRTESRKKKFLELYKTCKKESRDWKEELEDTEESKFRSKKLY |
| | | LYYTQKGRCMYSGEEIELKDLFNDNLYDIDHIYPRHFVKDDSIE |
| | | NNLVLVRKVDNAHKSDAVLESSIRNKMCGFWKILREGSFITDE |
| | | KYKRLMRTDDFSDEERAGFISRQIVETRQGTKVIADLFTKTFPE |
| | | SEIIYVKAGNVSDFRKKFDFLKCRIVNDFHHAQDAYLNIVVGN |
| | | VYNTKFTQNPMNFIREYNKDPKANKYHMDKLFEYPVSRNGRD |
| | | AWVTKNAESICMVKKMMEKNTPLITRMNYESHGGIADQTIYS |
| | | AEDAQKAKGKGYISIKSSDGNLLDTCKYGGMKKYTGTYFLLLE |
| | | HTKKEKRIRTIEAMPLYLKDKLNTREKIEKYFEQSVDFAYEQPK |
| | | VILDKIKMYSLIKVDGFYLYLTGRTGNQLSVCNAVQMSLSAGM |
| | | VKYVRKISIANERYAADKEFEKDENISVQKNLELYEILKEKHLN |
| | | QIYGKRPNPVGEKIEKGIEKFRSLSVRKQIYVLLQILQLSQLSNQ |
| | | GADLLEIGGEKKTGTSKLNKNITEKQEFKLINQSVTGLYENEID |
| | | LLRI |
| GEBx0321 | 25 | MSTMSYILGLDLGVASVGWSVIEIDENEMPIRLVDLGVRTFEKA |
| | | EVPKTGESLAKSRREARSIRRLIARRVRRLLLAKKALLEEGILTQ |
| | | NDFLTKSIIKDLPINAWELRAKGLDHKLTSKEWATVLLHLVKHR |
| | | GYLSQRKNESDTADKQLGALLSGVKENHTLLQENKYRTPAELA |
| | | ICKFEKEEGHIRNQNGEYIHTFDRLDLQAELHLLFEKQRQFNNP |
| | | YASAKLEEKMDYLLMYQKPALSGEAILKMLGKCSFETQEYKC |
| | | AKNTYSAERFIWLTKLNNLRILTQGSERGLTESERALLLNQPYQ |
| | | KAKLTYTQVRNILELSESEIFKGLRYGNVEDKKMIEKETLMELK |
| | | AFHQIRKVLEKANLKTEWQGLATKPELLDEIGTIFSLYKTDADIT |
| | | AQLQDKISAPILNALLSGINFDKFINLSLVALSKILPLMEQGQRYD |
| | | EACTSIYGDHYGVKTKEEHLFLPSIDKDEIRNPVVLRCLSQTRKI |
| | | INAVIRLYGSPARIHIETGREVGKSFKERQKIEKNQLENKAKKER |
| | | AIKEFKDLFPYFIGEPKSKDILKMRLYNEQQGKCLYSGKTIDINR |
| | | LLEKGYVEIDHALPFSRTWDDSFNNKVLVLANENQNKANQTPY |
| | | EWFDGKNNTTKWQQFVALVWGSHFNYHKKQHLLLKNLPEGF |
| | | MKRNLNDTRYVARYLMNFINDHIQLTGKGKRRVFASNGQITAL |
| | | LRARWGLMKVRGDNDRHHALDAIVVACTTPSMQNKITDFVRS |
| | | KEMNIFSGEFIDRNTGEIKQIHFPEPWNYFRNEVMIRVFSHDPLL |
| | | ELEQQLPSRPEARHQYVTPLFVSRMPTRKMTGQGHLETIKSAK |
| | | RLAENESVQRIALTSLKLNTLENMANKDKEKALYQALKERLEQ |
| | | FNNDPVKAFAEPFYKKGGQQVKKIRVKQIQKTGVIVHQGNGVA |
| | | DNASMVRVDVFEKANKFYLVPIYTWQVAKGILPNKAAIANKNE |
| | | DEWQEMDESFTFKFSIHPNDLIKVTTKKEVYFGYYVAMDRHTA |
| | | AINIREPDLERSKGKDGLHRGIGVKTALSFEKYDIDPLGKNIRPC |
| | | QPKARQPIR |
| GEBx0326 | 29 | MKQLVLGLDIGITSVGYGIIDIENNEIVDCGVRLFKEGSAAENET |
| | | RREKRGSRRLKSRKVNRIQDMENLLKELGIYENCNLIHNNPYD |
| | | LRIKGLSEKLTNKELTRVCLHYAKRRGSSLDMVEDDETKAKEL |
| | | GETKKILQENDILLKQGYYVCQVQKMNEQKYGKIHGNYNNFR |
| | | TSDYLKEAKQLLSIQGLKEENIEKILNIIQRKRQYYEGPGSLKSP |
| | | TIYGRFIEEDGRIIKIDLIEKMRGHCSIYPEELRAPKMSYSAELFN |
| | | FLNDLNNLKIDEEKITPEQKQEIISIIQTKKNITPLQLSKFLGVEIE |
| | | KISGFRIDKKDKPILTEFKGFKIIAKVLEKNNEKELVKDVKFVDS |
| | | ISEILTRTKDIQERIESIEKLNDNLSKECIYSLANITGIRGYHSLSL |
| | | KAIYEMNEELLNTEMNQMQILQSSKKLEQKINQKGKKNIQEDN |
| | | TAILSPVAKRAIREAIKVVNAARKRYGEFDSIVVETTRDKNSKE |
| | | QKKRIDDAQKYFKKRREEVDEVVNGRPLNAKTREKIRLYMDQ |
| | | DGKCAYSLEPIDLNLIMNDPTAYEIDHIIPISISLDDSFNNKVLVR |
| | | RKANQDKGNETPYNAIKQGKIKNKNLKSVSIQEYQAYVLNLRK |
| | | ENKISNKKKEYLLSQKDYTKLDVRKEFVNRNLIDTSYANRVVF |
| | | NTLEDYFKQNEIDTKVHTIRGAATSAFRKKVNLQKVREEDHSH |
| | | HAIDALIVASIKKLNLLNKLLDVKNIKDVYDDETGEYLPMDDN |
| | | SYFDPKYMNFINQLKDVKITKFSHKIDTKPNRQIADDTIYSTRIIE |
| | | GEEKLIKKYGDIYDPKFDKLTNDILNESYQNKYLMAIHDSKTFD |
| | | KFINIVQDHYNTYKDDSKIYTLDKGKIKLKGDNPLFLYKNEHGF |
| | | VTKYSKKNNGPAITQMKYVDCTLGNRIDITSNYKIKDDKKVVL |
| | | QQISPYRTDFYLDEDGHYQFVTVRYSNIKWKATQQSYIIDKEW |
| | | YKNAKKQKGIQENAKFCFSMHHDELIGFIKEDGEKLIYNDEHN |
| | | LINIMKEHNGKDYEILKFTATNNEGKNIVEVKPIYKYTSDRIKPL |
| | | INKKIIKLAKFATDPLGNLYEVKDSKLKLEL |
| GEBx0328 | 31 | MDKKPPYYIGLDCGTNSVGWAVSDKNYHLLKGTHHIKTEKGT |
| | | KTKKQTLWGFRLFEEAETAAERRIHRSARRRAERAKNRLKLLR |
| | | MLFHDEIVQVDPEFYQRLKESFYLLDDKKLQKNSKNTLFNDSN |
| | | LTDKDFHKKYPTIWHLRQAIINAKDDEAFDIRLYFLAIQHILKHR |
| | | GHFLMEGEINSDGDVDFESIFENFRCEAERVGYNIADVPAEVKE |
| | | LLMQKMSKTDKKKSLKDILYIEGDELDDDVPKGQLELAGLLVG |
| | | SKVDLKKIFSINDDEKFPYSFSEGAFEDKLPDIEMTVGSENLDLI |
| | | FAAKKIYDYVTLDNLLAGNKLISDAMVANYNQHSNDLKKLKQ |
| | | VFRPHREVYDKLFKTKLYNEKFPSYDAYISKAYTEDKSGRKSS |
| | | KNIGQEDFNKHLHKLLEEIKTKTKPELDSLVDELLDRTDTSEKS |
| | | VSPLLLPKQRGQAKGTIPQQLHGKELKYILQKLQKDYPSFAEVT |
| | | PGEDESCNTKAKKIIKIHEFRIPYYCGPIVKRKFDDDGKQVANG |
| | | KSQFSWADEEIAELVYPWNFDKLIKKEQRANNFIRRMTNECTH |
| | | LVGEDVLPKCSLIYQKYMILNELNNLKINGYRIDNQLKQQIFER |
| | | GYKNNEIKGNITLKKLEKWCKDNALISINDEMSGTSEVKTLPKY |
| | | QTYQDFSRILGTDFAHKYSANKLEKVVESITVLGDEKSMLKQSI |
| | | QEILNCDAKTANSLSKLSYKDWGRMSAKFLNGITVNDRTILDY |
| | | LWEDNKNLMELLGQEVGFGKIVEEYNKSKRPVTNRITYHDVES |
| | | LYCSPAVKRSVWQTIRIVDELVKNLQYAPEKIFLEVTRGEDKNA |
| | | KGKYTLARRKDLETKLKAVKTDDARAILKELDGYEDRDLQSK |
| | | KLFLYFSQMGKCAYSGEPISLEELNNSQLYDIDHIYPRSKTKDD |
| | | SITRNLVLVKANLNREKTNNYPISDTIRNKMHGIWSVWYHQGL |
| | | ITKEKYERLTRATPLTNDELGSFIARQLVETSQSVKAIRDLLKQA |
| | | YPKTKIVMVKAGQVSNFRHLMSRDKKDRDGNIYEPGKPEFIKV |
| | | REINDLHHAKDAYLNIVVGNVMNETFTDNPNEWIKRRAGKEY |
| | | SINPERIFRASKTYRKANGEETSYPEVNGWNFAESIQAVSNTMR |
| | | RNDVIWTRMNYIESGEISDLQLVGKSNKADGLLRIKQQERLDP |
| | | KKYGGYNSLKGAHFALIECKDKKGNLQRCIVQIPIIAKNNIENYI |
| | | AENYTDAQVILPIIKYKSLLKADGLLLTISNKDSDLKNGYQHAV |
| | | QAKFQQSISPYLKTICSICAKDKLARNNYQISTEDNVNVEHNLQ |
| | | LFDEFCDKMKIYSGYPELAGGIEKIMLSRTTFSELDLKQQCLTL |
| | | GQMMFIFACKAQNAKLNTIGCGDSVGRIRISNNKLFSYNNLHLI |
| | | NQSPTGLYEEVIDLKTVRPKVNKQDRE |
| GEBx0361 | 60 | MERYYLGLDMGTSSVGWAVTDEKYQIVRKKGKDLWGVRLFPE |
| | | AQTAAERRTNRVARRRRQREKARIGYLRELFANEIQKTDPGFFQ |
| | | RLDDSKYFQEDKMIHQPFALFADSGYTDKEYFQEYPTIYHLRKE |
| | | LLTSTEPHDVRLVYLAVLNMFKHRGHFLNASLTDESIGNLADLF |
| | | ENLRIAVKDQLDVELAYPERMEDLENIFIDKSLSKTEKVEKIKAL |
| | | LKIDKKDRLQAELWKMICGLSGTLSVIFRQDNFSEEDKKYKLSF |
| | | RDGNYDEKINAVAEMLSEESLDVLMLIKQIHDWGVLADIMRGS |
| | | GKTYEYLSFARVDSYEKHKKDLETLKYLYKTYAPEKYHDMFRV |
| | | MKDNNYSAYVGSVNAGNHVERRGYKHKTEDFYSALKKELQN |
| | | FPESAERQYVLNEIEKGTFLPKQLTASNGVVPNQVHMKELKKIL |
| | | ANAEKYLAFLQQTDETGLSVSEKIIQLFGFQIPYYVGPLVNKAD |
| | | NHAWVVRKEGGRVFPWNFEQKVDVKRTSELFIQNMVNHCTYL |
| | | SDEYVLPKNSLLYEKFMVLNELNNLRINGEKISVALKQDIYQDL |
| | | FKSGKKVTAKRLCDYLIVHGYVDNETPIDLSGIDGDFVNRLAN |
| | | YKKFSEVFYVETLTYEQEQMAEQIIFWSTVYGDSKKFLREKIQE |
| | | NYGEVLSSNQIKRILGYKFRDWGRLSREFLLLEGADKTTGEISTI |
| | | ISRMWNENYNLMQLLSSEQFTYREEVEKKTKHMEKTLSEITYE |
| | | DLEDLYISAPVRRMTWQTILILKELCGVLGQYPDKVFVEMARE |
| | | HGEKNTRTVSRKKKFMDLYKSCKDEEIQWAKQIADTEERKFRS |
| | | KKLYLYYTQKGRCMYTGTRIELSDLFNDNLYDIDHIYPRHFVKD |
| | | DNIDNNLVLVKKQVNAHKSDVFPLERDIQKKQAAMWKELRQG |
| | | NFITEEKYKRLTRNDPFTEEEQAGFISRQIVETRQGTKAITDLLQ |
| | | QTCPQSKIIYVKAGNVSAFRQKFDLIKCRNVNDLHHAQDAYLNI |
| | | VVGNVYYTKFTSSPRNFVREYRQNKEKHPYHMYRLFDYDVRR |
| | | GEEVAWEIGKDGKGKSISTVKRMVLKNTPLVTMMNFEVHGGL |
| | | ADQTLYSAKDAKDGEGYLPLKSSDPRLRDVAKYGGFTKYTGT |
| | | YFFVVEYGQEKKRIRSIEAVPLYLAERLSDRENLEVYCRDMLGY |
| | | QDPKIICRKIKMYSLMKINGFYLYLTGRTGTRLNVVNAVQMILD |
| | | YENMYYVKRIADASERKYTDEDYEEHEVISKENNLRLYRQLEE |
| | | KHRDTIYQKRPNPMGSKLQEMEGRFEELSIAQQVKVLLEILKLS |
| | | QRLNQGADLTLLGGAKKTGTSKIGKKISELQECKVINQSVTGM |
| | | YVNEVDLLKK |
| N-terminal NLS | 881 | MAPKKKRKV |
| C-terminal NLS | 882 | KRPAATKKAGQAKKKK |
| FLAG sequence | 883 | DYKDDDDK |

TABLE 2
The exemplary polynucleotides encoding Cas proteins

| | SEQ | | SEQ | | SEQ |
|---|---|---|---|---|---|
| Name | ID NOs: | Name | ID NOs: | Name | ID NOs: |
| GEBx0293 | 161 | GEBx0321 | 185 | GEBx0347 | 209 |
| GEBx0294 | 162 | GEBx0323 | 186 | GEBx0348 | 210 |
| GEBx0295 | 163 | GEBx0324 | 187 | GEBx0351 | 211 |
| GEBx0296 | 164 | GEBx0325 | 188 | GEBx0352 | 212 |
| GEBx0299 | 165 | GEBx0326 | 189 | GEBx0353 | 213 |
| GEBx0300 | 166 | GEBx0327 | 190 | GEBx0354 | 214 |
| GEBx0301 | 167 | GEBx0328 | 191 | GEBx0356 | 215 |
| GEBx0303 | 168 | GEBx0329 | 192 | GEBx0357 | 216 |
| GEBx0305 | 169 | GEBx0330 | 193 | GEBx0358 | 217 |
| GEBx0306 | 170 | GEBx0331 | 194 | GEBx0359 | 218 |
| GEBx0307 | 171 | GEBx0332 | 195 | GEBx0360 | 219 |
| GEBx0308 | 172 | GEBx0333 | 196 | GEBx0361 | 220 |
| GEBx0309 | 173 | GEBx0334 | 197 | GEBx0363 | 221 |
| GEBx0310 | 174 | GEBx0335 | 198 | GEBx0364 | 222 |
| GEBx0311 | 175 | GEBx0336 | 199 | GEBx0375 | 223 |
| GEBx0312 | 176 | GEBx0337 | 200 | GEBx0377 | 224 |
| GEBx0313 | 177 | GEBx0338 | 201 | GEBx0378 | 225 |
| GEBx0314 | 178 | GEBx0340 | 202 | GEBx0379 | 226 |
| GEBx0315 | 179 | GEBx0341 | 203 | GEBx0380 | 227 |
| GEBx0316 | 180 | GEBx0342 | 204 | GEBx0381 | 228 |
| GEBx0317 | 181 | GEBx0343 | 205 | GEBx0382 | 229 |
| GEBx0318 | 182 | GEBx0344 | 206 | GEBx0383 | 230 |
| GEBx0319 | 183 | GEBx0345 | 207 | GEBx0384 | 231 |
| GEBx0320 | 184 | GEBx0346 | 208 | | |
| GEBx0293 with | 241 | GEBx0321 with | 265 | GEBx0347 with | 289 |
| sequence encoding | | sequence encoding | | sequence encoding | |
| NLS and Flag-tag | | NLS and Flag-tag | | NLS and Flag-tag | |
| GEBx0294 with | 242 | GEBx0323 with | 266 | GEBx0348 with | 290 |
| sequence encoding | | sequence encoding | | sequence encoding | |
| NLS and Flag-tag | | NLS and Flag-tag | | NLS and Flag-tag | |
| GEBx0295 with | 243 | GEBx0324 with | 267 | GEBx0351 with | 291 |
| sequence encoding | | sequence encoding | | sequence encoding | |
| NLS and Flag-tag | | NLS and Flag-tag | | NLS and Flag-tag | |
| GEBx0296 with | 244 | GEBx0325 with | 268 | GEBx0352 with | 292 |
| sequence encoding | | sequence encoding | | sequence encoding | |
| NLS and Flag-tag | | NLS and Flag-tag | | NLS and Flag-tag | |
| GEBx0299 with | 245 | GEBx0326 with | 269 | GEBx0353 with | 293 |
| sequence encoding | | sequence encoding | | sequence encoding | |
| NLS and Flag-tag | | NLS and Flag-tag | | NLS and Flag-tag | |
| GEBx0300 with | 246 | GEBx0327 with | 270 | GEBx0354 with | 294 |
| sequence encoding | | sequence encoding | | sequence encoding | |
| NLS and Flag-tag | | NLS and Flag-tag | | NLS and Flag-tag | |
| GEBx0301 with | 247 | GEBx0328 with | 271 | GEBx0356 with | 295 |
| sequence encoding | | sequence encoding | | sequence encoding | |
| NLS and Flag-tag | | NLS and Flag-tag | | NLS and Flag-tag | |
| GEBx0303 with | 248 | GEBx0329 with | 272 | GEBx0357 with | 296 |
| sequence encoding | | sequence encoding | | sequence encoding | |
| NLS and Flag-tag | | NLS and Flag-tag | | NLS and Flag-tag | |
| GEBx0305 with | 249 | GEBx0330 with | 273 | GEBx0358 with | 297 |
| sequence encoding | | sequence encoding | | sequence encoding | |
| NLS and Flag-tag | | NLS and Flag-tag | | NLS and Flag-tag | |
| GEBx0306 with | 250 | GEBx0331 with | 274 | GEBx0359 with | 298 |
| sequence encoding | | sequence encoding | | sequence encoding | |
| NLS and Flag-tag | | NLS and Flag-tag | | NLS and Flag-tag | |
| GEBx0307 with | 251 | GEBx0332 with | 275 | GEBx0360 with | 299 |
| sequence encoding | | sequence encoding | | sequence encoding | |
| NLS and Flag-tag | | NLS and Flag-tag | | NLS and Flag-tag | |
| GEBx0308 with | 252 | GEBx0333 with | 276 | GEBx0361 with | 300 |
| sequence encoding | | sequence encoding | | sequence encoding | |
| NLS and Flag-tag | | NLS and Flag-tag | | NLS and Flag-tag | |
| GEBx0309 with | 253 | GEBx0334 with | 277 | GEBx0363 with | 301 |
| sequence encoding | | sequence encoding | | sequence encoding | |
| NLS and Flag-tag | | NLS and Flag-tag | | NLS and Flag-tag | |
| GEBx0310 with | 254 | GEBx0335 with | 278 | GEBx0364 with | 302 |
| sequence encoding | | sequence encoding | | sequence encoding | |
| NLS and Flag-tag | | NLS and Flag-tag | | NLS and Flag-tag | |
| GEBx0311 with | 255 | GEBx0336 with | 279 | GEBx0375 with | 303 |
| sequence encoding | | sequence encoding | | sequence encoding | |
| NLS and Flag-tag | | NLS and Flag-tag | | NLS and Flag-tag | |
| GEBx0312 with | 256 | GEBx0337 with | 280 | GEBx0377 with | 304 |
| sequence encoding | | sequence encoding | | sequence encoding | |
| NLS and Flag-tag | | NLS and Flag-tag | | NLS and Flag-tag | |
| GEBx0313 with | 257 | GEBx0338 with | 281 | GEBx0378 with | 305 |
| sequence encoding | | sequence encoding | | sequence encoding | |
| NLS and Flag-tag | | NLS and Flag-tag | | NLS and Flag-tag | |
| GEBx0314 with | 258 | GEBx0340 with | 282 | GEBx0379 with | 306 |
| sequence encoding | | sequence encoding | | sequence encoding | |
| NLS and Flag-tag | | NLS and Flag-tag | | NLS and Flag-tag | |
| GEBx0315 with | 259 | GEBx0341 with | 283 | GEBx0380 with | 307 |
| sequence encoding | | sequence encoding | | sequence encoding | |
| NLS and Flag-tag | | NLS and Flag-tag | | NLS and Flag-tag | |
| GEBx0316 with | 260 | GEBx0342 with | 284 | GEBx0381 with | 308 |
| sequence encoding | | sequence encoding | | sequence encoding | |
| NLS and Flag-tag | | NLS and Flag-tag | | NLS and Flag-tag | |
| GEBx0317 with | 261 | GEBx0343 with | 285 | GEBx0382 with | 309 |
| sequence encoding | | sequence encoding | | sequence encoding | |
| NLS and Flag-tag | | NLS and Flag-tag | | NLS and Flag-tag | |
| GEBx0318 with | 262 | GEBx0344 with | 286 | GEBx0383 with | 310 |
| sequence encoding | | sequence encoding | | sequence encoding | |
| NLS and Flag-tag | | NLS and Flag-tag | | NLS and Flag-tag | |
| GEBx0319 with | 263 | GEBx0345 with | 287 | GEBx0384 with | 311 |
| sequence encoding | | sequence encoding | | sequence encoding | |
| NLS and Flag-tag | | NLS and Flag-tag | | NLS and Flag-tag | |
| GEBx0320 with | 264 | GEBx0346 with | 288 | | |
| sequence encoding | | sequence encoding | | | |
| NLS and Flag-tag | | NLS and Flag-tag | | | |

TABLE 3
The exemplary direct repeat sequences described in the disclosure

| Name | SEQ ID NOs: |
|---|---|
| GEBx0293-DR | 341 |
| GEBx0294-DR | 342 |
| GEBx0295-DR | 343 |
| GEBx0296-DR | 344 |
| GEBx0299-DR | 345 |
| GEBx0300-DR | 346 |
| GEBx0301-DR | 347 |
| GEBx0303-DR | 348 |
| GEBx0305-DR | 349 |
| GEBx0306-DR | 350 |
| GEBx0307-DR | 351 |
| GEBx0308-DR | 352 |
| GEBx0309-DR | 353 |
| GEBx0310-DR | 354 |
| GEBx0311-DR | 355 |
| GEBx0312-DR | 356 |
| GEBx0313-DR | 357 |
| GEBx0314-DR | 358 |
| GEBx0315-DR | 359 |
| GEBx0316-DR | 360 |
| GEBx0317-DR | 361 |
| GEBx0318-DR | 362 |
| GEBx0319-DR | 363 |
| GEBx0323-DR | 364 |
| GEBx0324-DR | 365 |
| GEBx0325-DR | 366 |
| GEBx0326-DR | 367 |
| GEBx0327-DR | 368 |
| GEBx0328-DR | 369 |
| GEBx0329-DR | 370 |
| GEBx0330-DR | 371 |
| GEBx0331-DR | 372 |
| GEBx0332-DR | 373 |
| GEBx0333-DR | 374 |
| GEBx0334-DR | 375 |
| GEBx0335-DR | 376 |
| GEBx0336-DR | 377 |
| GEBx337-HPT-V1-DR | 378 |
| GEBx338-HPT-V1-DR | 379 |
| GEBx339-HPT-V1-DR | 380 |
| GEBx340-HPT-V1-DR | 381 |
| GEBx341-HPT-V1-DR | 382 |
| GEBx342-HPT-V1-DR | 383 |
| GEBx343-HPT-V1-DR | 384 |
| GEBx345-HPT-V1-DR | 385 |
| GEBx346-HPT-V1-DR | 386 |
| GEBx347-HPT-V1-DR | 387 |
| GEBx351-HPT-V1-DR | 388 |
| GEBx352-HPT-V1-DR | 389 |
| GEBx353-HPT-V1-DR | 390 |
| GEBx354-HPT-V1-DR | 391 |
| GEBx355-HPT-V1-DR | 392 |
| GEBx355-HPT-V2-DR | 393 |
| GEBx355-HPT-V3-DR | 394 |
| GEBx356-HPT-V1-DR | 395 |
| GEBx357-HPT-V1-DR | 396 |
| GEBx357-HPT-V2-DR | 397 |
| GEBx357-HPT-V3-DR | 398 |
| GEBx359-HPT-V1-DR | 399 |
| GEBx360-HPT-V1-DR | 400 |
| GEBx360-HPT-V2-DR | 401 |
| GEBx0361-HPT-V1-DR | 402 |
| GEBx0361-HPT-V2-DR | 403 |
| GEBx362-HPT-V1-DR | 404 |
| GEBx362-HPT-V2-DR | 405 |
| GEBx363-HPT-V1-DR | 406 |
| GEBx363-HPT-V2-DR | 407 |
| GEBx364-HPT-V1-DR | 408 |
| GEBx375-HPT-V1-DR | 409 |
| GEBx375-HPT-V2-DR | 410 |
| GEBx377-HPT-V1-DR | 411 |
| GEBx378-HPT-V1-DR | 412 |
| GEBx379-HPT-V1-DR | 413 |
| GEBx381-HPT-V1-DR | 414 |
| GEBx382-HPT-V1-DR | 415 |
| GEBx383-HPT-V1-DR | 416 |
| GEBx384-HPT-V1-DR | 417 |

In some embodiments, this disclosure provides an engineered, non-naturally occurring crRNA, wherein the crRNA comprises a nucleotide sequence having at least 90% sequence identity to any one of SEQ ID NOs: 341-417 (Table 3), or a variant thereof. In some embodiments, the crRNA comprises a nucleotide sequence having at least 95% or 98% sequence identity to any one of SEQ ID NOs: 341-417. In some embodiments, the crRNA comprises a nucleotide sequence set forth in any one of SEQ ID NOs: 341-417.

The following non-limiting examples are provided to further illustrate embodiments of the disclosure disclosed herein. It should be appreciated by those of skill in the art that the techniques disclosed in the examples that follow represent approaches that have been found to function well in the practice of the disclosure, and thus can be considered to constitute examples of modes for its practice. Those of skill in the art should, in light of the present disclosure, appreciate that many changes can be made in the specific embodiments that are disclosed and still obtain a like or similar result without departing from the spirit and scope of the disclosure.

### Example 1: a Method of Metagenomic Analysis for the Proteins

Metagenomic sequence data from public databases were search using Hidden Markov Models generated based on known Cas protein sequences including class 2 type II Cas effector proteins. CRISPR-Cas protein identified by the search are aligned to known proteins to identify potential active sites. After screening through hundreds of potential sequences, this metagenomic workflow results in the delineation of the type II Cas protein as detailed in Table 1.

The phylogenetic tree was generated using MUSCLE 3 (Veen et al., 2020) to explore the relationships among orthologs at the primary amino-acid level. This exploration made use of hundreds of Class 2 Type II-A/B/C sequences sourced from the National Center for Biotechnology Information (NCBI), as well as from various publications and patents. Notably, the phylogenetic tree suggests that the Cas protein described in this invention is distinct from those previously known. The Type II Cas proteins detailed in this disclosure share only a low degree of identity with other known Cas proteins.

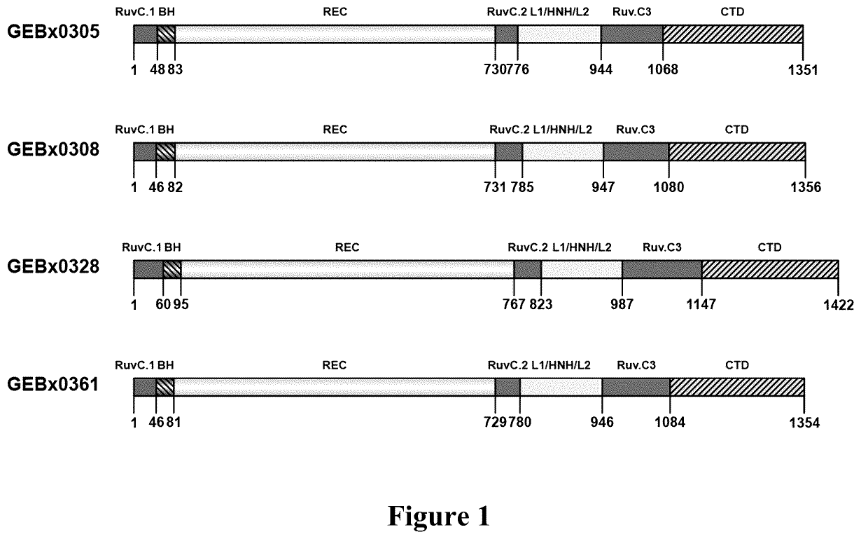

The structural modeling of the Type II Cas protein was accomplished using AlphaFold2. The domain arrangement was annotated, revealing that the Cas protein disclosed in this invention comprises a RuvC domain, a BH (bridge helix) domain, a REC domain, an HNH domain, and a CTD (C-terminal domain). Notably, the RuvC domain includes three distinct RuvC sub-domains, along with the BH domain. FIG. 1 provides a visual representation of some exemplary proteins' structure.

### Example 2: Protocol for Predicted RNA Folding

Predicted RNA folding of the putative guide RNA sequences of the Cas proteins were computed using the RNAfold webserver developed by Lorenz et al 2011.

### Example 3: PAM Determination in Mammalian Cell Line

In a set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 450 μL of cells with a density of 120,000 cells/well was mixed with 50 μL mixture containing Lipofectamine™ 3000 (ThermoFisher Scientific, Cat. L3000008), Opti-Mem (Volume refill to 50 L), 1 μL dsODN (2.5 pM), 100 ng (˜1 μL) psgRNA harbored sgRNA scaffold (Table 6) and Humanspacer3 spacer (SEQ ID NO: 885, Table 4) encoding sequences and 400 ng (˜1 L) pCas protein plasmid harbored Cas9 CDS with NLS and FLAG encoding sequences (Table 2) per the manufacturer's protocol. Then seeded the cell mixture onto a 24-well plate and cultured at 37° C. and 5% CO₂. 10 pM dsODN was annealed using dsODN-Top and dsODN-BoT oligonucleotides pre-transfection.

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8-2.0) were subjected to Guide-Seq NGS analyses.

The basic method of Guide-Seq library preparation is described by Nikolay et. al (Nat. Protoc. 2021). The extracted DNA sample were first sheared using KAPAFrag Kit (Cat #KK8602, Roche). Fragmented DNA was purified and then phosphorated using T4 Polynucleotide Kinase (Cat #M0201S, NEB). An SS5-adapter (generated by annealing 10 M SS5TOP oligo with 10 μM SS5B™ oligo) was ligated to the fragmented DNA using Quick Ligation™ Kit (Cat #M2200S, NEB), followed by two steps off-target PCR to add chemistry for sequencing.

For off-target PCR1 was performed using Platinum™ Taq DNA Polymerase (Cat #15966005, Invitrogen) with GSP1 (a mixture of GSP1-Top and GSP1-BoT) and Y_XX oligos. For off-target PCR2 was performed using Platinum™ Taq DNA Polymerase with GSP2 (a mixture of GSP2-TopA/B/C and GSP1-BoTA/B/C), Y_XX (Same to PCR1) and i753_XX oligos. The DNA product in each step described above need purification using SPRI Select (Cat #B23318, Beckman Coulter). The final library was quantified with PCR and sequenced on Illumina NextSeq 1000. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected.

TABLE 4
The nucleotide sequences referred to in the example.

| | SEQ ID | |
|---|---|---|
| Name | NOs: | 5′-3′ |
| Humanspacer3 | 885 | GTGAGCCACTGTGCCTGGCC |
| dsODN-Top | 886 | pACTTCGGTACGCGTCGACGTACGGTCGACGAGTCGCGCGTACGAC |
| | | GATCGA*G*A*C |
| dsODN-BOT | 887 | pGTCTCGATCGTCGTACGCGCGACTCGTCGACCGTACGTCGACGCG |
| | | TACCGA*A*G*T |
| SS5TOP | 888 | A*A*C*TACACGACGCTCTTCCGATCTNNNWNNNWNNAGTGACAC |
| SS5BTM | 889 | NNNNNNGTGTCACTNNWNNNWNNNAGATCGGAAGAGCGTCGTGT |
| | | AGTTp |
| GSP1-Top | 890 | GTCGACGAGTCGCGCGTA |
| GSP1-BoT | 891 | TCGTCGACCGTACGTCGA |
| GSP2-TopA | 892 | GTGACTGGAGTTCAGACGTGTGCTCTTCCGATCTCGCGCGTACGAC |
| | | GAT*C*G*A |
| GSP2-TopB | 893 | GTGACTGGAGTTCAGACGTGTGCTCTTCCGATCTATCGCGCGTACG |
| | | ACGAT*C*G*A |
| GSP2-TopC | 894 | GTGACTGGAGTTCAGACGTGTGCTCTTCCGATCTTAACGCGCGTAC |
| | | GACGAT*C*G*A |
| GSP2-BoTA | 895 | GTGACTGGAGTTCAGACGTGTGCTCTTCCGATCTTACGTCGACGCG |
| | | TAC*C*G*A |
| GSP2-BoTB | 896 | GTGACTGGAGTTCAGACGTGTGCTCTTCCGATCTGCTACGTCGACG |
| | | CGTAC*C*G*A |
| GSP2-BoTC | 897 | GTGACTGGAGTTCAGACGTGTGCTCTTCCGATCTATGTACGTCGAC |
| | | GCGTAC*C*G*A |
| Y_XX | 898 | AATGATACGGCGACCACCGAGATCTACACNNNNNNNNACACTCTTT |
| | | CCCTACACGACGCTCTTCCGATCT |
| i753_XX | 899 | CAAGCAGAAGACGGCATACGAGATNNNNNNNNGTGACTGGAGTTC |
| | | AGACGTGTGCTCTTCCGATCT |

Note:
“p” denotes a phosphorylation modification; “*” denotes a phosphorothioate (PS) bond; “N” denotes any natural or non-natural nucleotide.

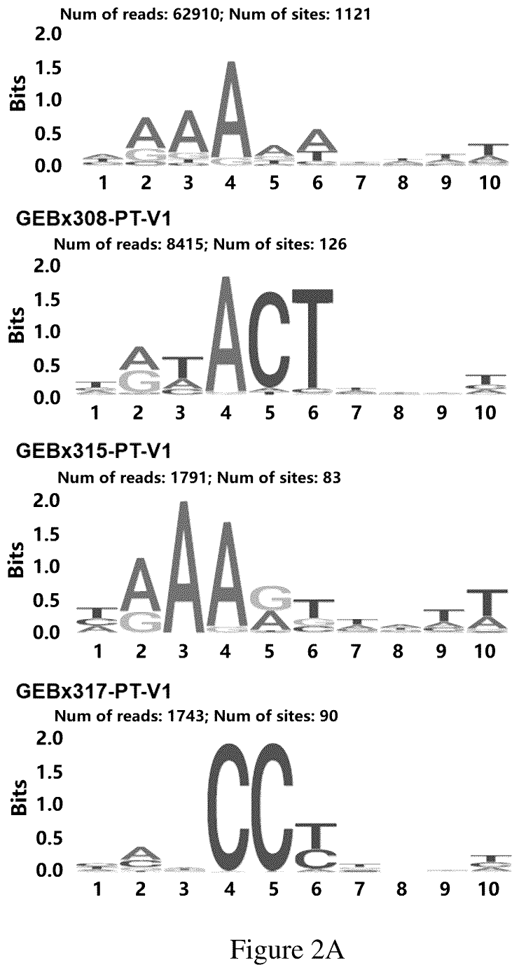

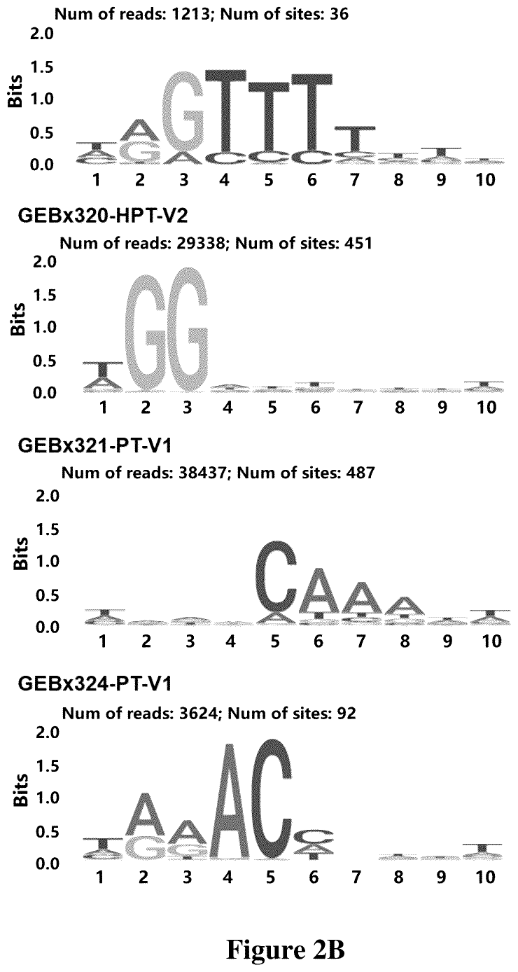

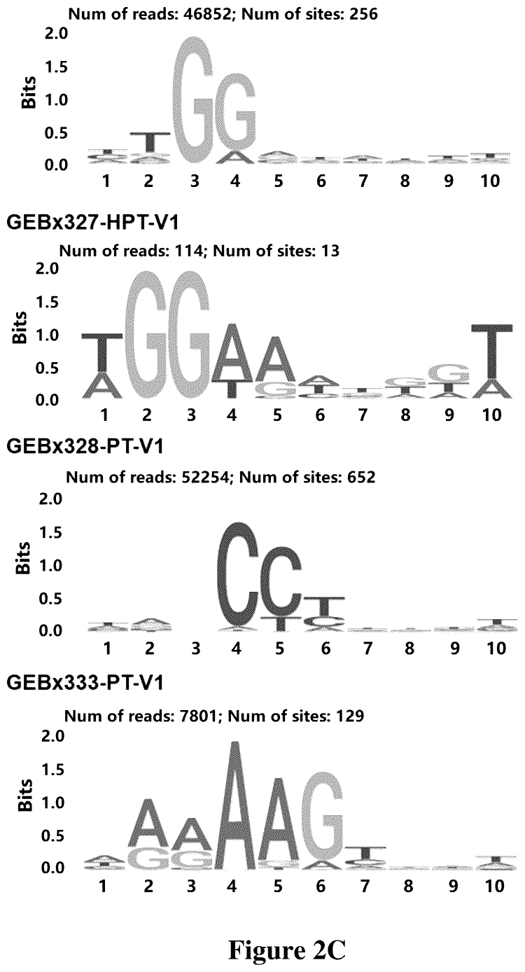

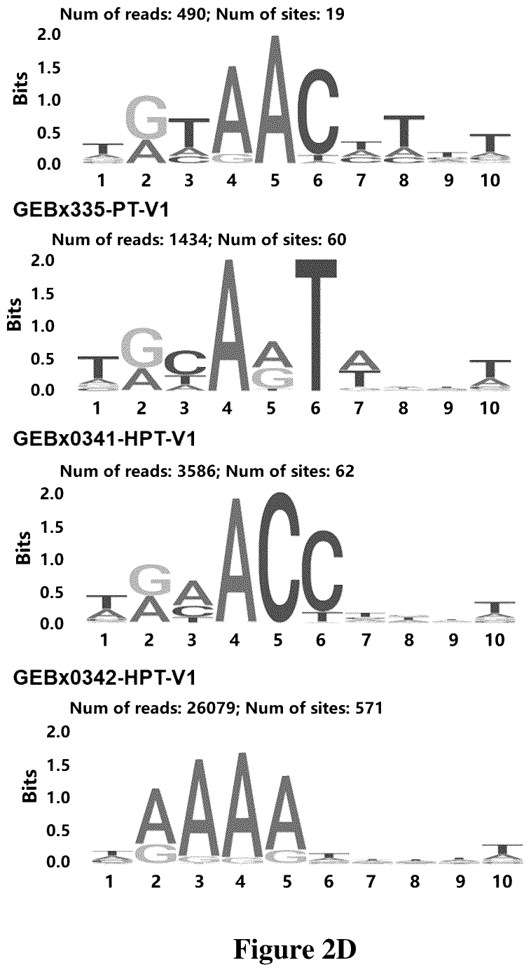

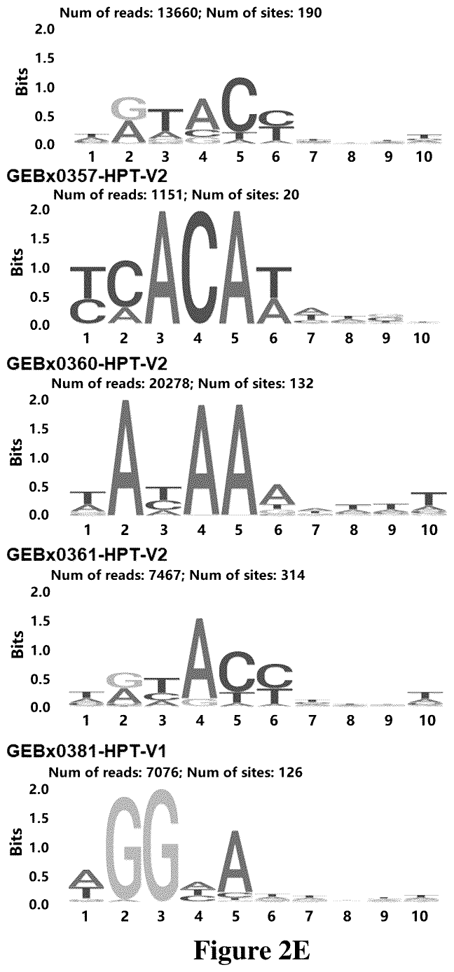

The PAM preference of some exemplary Cas proteins indicated in TEK293 cell line when using the corresponding sgRNA scaffold is shown in FIGS. 2A-2E and Table 5.

TABLE 5
The PAM preference of the exemplary Cas proteins

| Protein | Spacer | sgRNA scaffold | PAM sequence |
|---|---|---|---|
| GEBx0305 (SEQ ID NO: 89) | Humanspacer3 | GEBx0305-HPT-V1 | NRRANH |
| GEBx0308 (SEQ ID NO: 92) | SEQ ID NO: 885 | GEBx0308-PT-V1 | NRHACT |
| GEBx0315 (SEQ ID NO: 99) | | GEBx0315-PT-V1 | NRAAR |
| GEBx0317 (SEQ ID NO: 101) | | GEBx0317-PT-V1 | NNNCCY |
| GEBx0318 (SEQ ID NO: 102) | | GEBx0318-PT-V1 | NNRYYYY |
| GEBx0320 (SEQ ID NO: 104) | | GEBx0320-HPT-V2 | NGG |
| GEBx0321 (SEQ ID NO: 105) | | GEBx0321-PT-V1 | NNNNCAA |
| GEBx0324 (SEQ ID NO: 107) | | GEBx0324-PT-V1 | NRNAC |
| GEBx0326 (SEQ ID NO: 109) | | GEBx0326-HPT-V1 | NNGR |
| GEBx0327 (SEQ ID NO: 110) | | GEBx0327-HPT-V1 | NGGNR |
| GEBx0328 (SEQ ID NO: 111) | | GEBx0328-PT-V1 | NNNCCH |
| GEBx0333 (SEQ ID NO: 116) | | GEBx0333-PT-V1 | NRRAAG |
| GEBx0334 (SEQ ID NO: 117) | | GEBx0334-PT-V1 | NRHRAC |
| GEBx0335 (SEQ ID NO: 118) | | GEBx0335-PT-V1 | NRYART |
| GEBx0341 (SEQ ID NO: 123) | | GEBx0341-HPT-V1 | NRHACC |
| GEBx0342 (SEQ ID NO: 124) | | GEBx0342-HPT-V1 | NRAAR |
| GEBx0351 (SEQ ID NO: 131) | | GEBx0351-HPT-V1 | NRNVHH |
| GEBx0357 (SEQ ID NO: 136) | | GEBx0357-HPT-V2 | YMACAW |
| GEBx0360 (SEQ ID NO: 139) | | GEBx0360-HPT-V2 | NAHAA |
| GEBx0361 (SEQ ID NO: 140) | | GEBx0361-HPT-V2 | NRHAYY |
| GEBx0381 (SEQ ID NO: 148) | | GEBx0381-HPT-V1 | NGGHA |

### Example 4: In Vitro Editing Efficiency Screening of the CRISPR-Cas System in Mammalian Cell Line

In a set of experiments, the HTEK293T cells were cultured in DMVEM media supplemented with 1000 fetal bovine serum (Gibco™). For reverse transfection, the HEK293T cells were cultured in DMVEM media supplemented with 10% fetal bovine serum (Gibco™). A volume of 250 μL of cells with a density of 50,000 cells/well was seeded onto a 48-well plate 24 hours pre-transfection. Cells were transfected with a lipoplex containing Lipofectamine™ 3000 (0.4 μL/well), P3000 (2 μL/well), pgRNA/pCas protein plasmid (125 ng/well and 375 ng/well, respectively) and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example comprises sequences encoding the corresponding Cas sgRNA scaffold (Table 6; SEQ ID NO: 465 (GEBx0305), SEQ ID NO: 451 (GEBx0308), SEQ ID NO: 454 (GEBx0308-Rq-V3), SEQ ID NO: 478 (spCas9), SEQ ID NO: 491 and SEQ ID NO: 548) and the corresponding spacers (Table 7).

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8-2.0) were subjected to amplicons NGS analyses.

To quantitatively determine the efficiency of editing at the target location in the genome, NGS was utilized to identify the presence of insertions and deletions introduced by gene editing. Primers used for NGS which around the target area within the endogenous genes were designed. Additional PCR was performed per the manufacturer's protocols (Illumina) to add chemistry for sequencing. The amplicons were sequenced on Illumina iSeq 100. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an insertion, substitution, or deletion was calculated. The number of the reads mapped the reference genome is more than 1000.

TABLE 6
The sgRNA scaffold sequences of the corresponding Cas proteins.

| Name | SEQ ID NOs: |
|---|---|
| GEBx0299-Pt-V1 | 431 |
| GEBx0304-Pt-V1 | 432 |
| GEBx0308-Pt-V1 (WT) | 433 |
| GEBx0309-Pt-V1 | 434 |
| GEBx0310-Pt-V1 | 435 |
| GEBx0310-Pt-V2 | 436 |
| GEBx0314-Pt-V1 | 437 |
| GEBx0314-Pt-V2 | 438 |
| GEBx0315-Pt-V1 | 439 |
| GEBx0315-Rq | 440 |
| GEBx0316-Pt-V1 | 441 |
| GEBx0317-Pt-V1 | 442 |
| GEBx0318-Rq-V1 | 443 |
| GEBx0318-Rq-V2 | 444 |
| GEBx0318-Rq-V3 | 445 |
| GEBx0318-Rq-V4 | 446 |
| GEBx0321-Pt-V1 | 447 |
| GEBx0321-Pt-V2 | 448 |
| GEBx0309-Rq-V1 | 449 |
| GEBx0309-Rq-V2 | 450 |
| GEBx0308-PT-V1 (WT) | 451 |
| GEBx0308-Rq-V1 | 452 |
| GEBx0308-Rq-V2 | 453 |
| GEBx0308-Rq-V3 (M0) | 454 |
| GEBx0308-M1 | 455 |
| GEBx0308-M2 | 456 |
| GEBx0308-M3 | 457 |
| GEBx0308-M4 | 458 |
| GEBx0308-M5 | 459 |
| GEBx0308-M6 | 460 |
| GEBx0314-Rq-V1 | 461 |
| GEBx0295-HPT-V1 | 462 |
| GEBx0296-HPT-V1 | 463 |
| GEBx0297-HPT-V1 | 464 |
| GEBx0305-HPT-V1 (WT) | 465 |
| GEBx0305-Rq-V2 (M0) | 466 |
| GEBx0305-M1 | 467 |
| GEBx0305-M2 | 468 |
| GEBx0305-M3 | 469 |
| GEBx0305-M4 | 470 |
| GEBx0305-M5 | 471 |
| GEBx0305-M6 | 472 |
| GEBx0306-HPT-V1 | 473 |
| GEBx0313-HPT-V1 | 474 |
| GEBx0319-HPT-V1 | 475 |
| GEBx0320-HPT-V1 | 476 |
| GEBx0320-HPT-V2 | 477 |
| SpCas9 sgRNA in plasmid | 478 |
| GEBx0318-pt-v1 | 479 |
| GEBx0323-Rq-V1 | 480 |
| GEBx0324-PT-V1 | 481 |
| GEBx0324-HPT-V1 | 482 |
| GEBx0324-HPT-V1 | 483 |
| GEBx0325-PT-V1 | 484 |
| GEBx0325-HPT-V1 | 485 |
| GEBx0325-HPT-V2 | 486 |
| GEBx0326-HPT-V1 | 487 |
| GEBx0327-PT-V1 | 488 |
| GEBx0327-HPT-V1 | 489 |
| GEBx0327-HPT-V1 | 490 |
| GEBx0328-PT-V1 | 491 |
| GEBx0328-Rq-V1 | 492 |
| GEBx0328-M1 | 493 |
| GEBx0328-M2 | 494 |
| GEBx0328-M3 | 495 |
| GEBx0328-M4 | 496 |
| GEBx0328-M5 | 497 |
| GEBx0328-M6 | 498 |
| GEBx0328-M7 | 499 |
| GEBx0328-M8 | 500 |
| GEBx0329-PT-V1 | 501 |
| GEBx0329-HPT-V1 | 502 |
| GEBx0329-HPT-V2 | 503 |
| GEBx0330-PT-V1 | 504 |
| GEBx0330-HPT-V1 | 505 |
| GEBx0330-HPT-V2 | 506 |
| GEBx0332-PT-V1 | 507 |
| GEBx0332-HPT-V1 | 508 |
| GEBx0332-HPT-V2 | 509 |
| GEBx0333-PT-V1 | 510 |
| GEBx0333-HPT-V1 | 511 |
| GEBx0333-HPT-V2 | 512 |
| GEBx0334-PT-V1 | 513 |
| GEBx0334-HPT-V1 | 514 |
| GEBx0334-HPT-V2 | 515 |
| GEBx0335-PT-V1 | 516 |
| GEBx0335-HPT-V1 | 517 |
| GEBx0335-HPT-V2 | 518 |
| GEBx0331-HPT-V1 | 519 |
| GEBx0336-HPT-V1 | 520 |
| GEBx0336-HPT-V2 | 521 |
| GEBx0336-HPT-V3 | 522 |
| GEBx0337-HPT-V1 | 523 |
| GEBx0338-HPT-V1 | 524 |
| GEBx0339-HPT-V1 | 525 |
| GEBx0340-HPT-V1 | 526 |
| GEBx0341-HPT-V1 | 527 |
| GEBx0342-HPT-V1 | 528 |
| GEBx0343-HPT-V1 | 529 |
| GEBx0345-HPT-V1 | 530 |
| GEBx0346-HPT-V1 | 531 |
| GEBx0347-HPT-V1 | 532 |
| GEBx0351-HPT-V1 | 533 |
| GEBx0352-HPT-V1 | 534 |
| GEBx0353-HPT-V1 | 535 |
| GEBx0354-HPT-V1 | 536 |
| GEBx0355-HPT-V1 | 537 |
| GEBx0355-HPT-V2 | 538 |
| GEBx0355-HPT-V3 | 539 |
| GEBx0356-HPT-V1 | 540 |
| GEBx0357-HPT-V1 | 541 |
| GEBx0357-HPT-V2 | 542 |
| GEBx0357-HPT-V3 | 543 |
| GEBx0359-HPT-V1 | 544 |
| GEBx0360-HPT-V1 | 545 |
| GEBx0360-HPT-V2 | 546 |
| GEBx0361-HPT-V1 | 547 |
| GEBx0361-HPT-V2 | 548 |
| GEBx0362-HPT-V1 | 549 |
| GEBx0362-HPT-V2 | 550 |
| GEBx0363-HPT-V1 | 551 |
| GEBx0363-HPT-V2 | 552 |
| GEBx0364-HPT-V1 | 553 |
| GEBx0375-HPT-V1 | 554 |
| GEBx0375-HPT-V2 | 555 |
| GEBx0377-HPT-V1 | 556 |
| GEBx0378-HPT-V1 | 557 |
| GEBx0379-HPT-V1 | 558 |
| GEBx0381-HPT-V1 | 559 |
| GEBx0382-HPT-V1 | 560 |
| GEBx0383-HPT-V1 | 561 |
| GEBx0384-HPT-V1 | 562 |

The spacer sequence is located at the 5′ end of the scaffold. And the 3′ end of each spacer sequence is directly linked to the 5′ end of the subsequent scaffold sequence, forming the characteristic repeat-spacer pattern.

TABLE 7
The exemplary spacer sequences

| SEQ | |
|---|---|
| ID | Name or description |
| NOs | of the spacer |
| 571 | CFTR-NGGAAAA-T1 for |
| | GEBx0305 |
| 572 | CFTR-NGGAAAA-T1 for |
| | SpCas9 |
| 573 | CFTR-NGGAAAA-T2 for |
| | GEBx0305 |
| 574 | CFTR-NGGAAAA-T2 for |
| | SpCas9 |
| 575 | CFTR-NGGAAAA-T3 for |
| | GEBx0305 |
| 576 | CFTR-NGGAAAA-T3 for |
| | SpCas9 |
| 577 | CFTR-NGGAAAA-T4 for |
| | GEBx0305 |
| 578 | CFTR-NGGAAAA-T4 for |
| | SpCas9 |
| 579 | CFTR-NGGAAAA-T5 for |
| | GEBx0305 |
| 580 | CFTR-NGGAAAA-T5 for |
| | SpCas9 |
| 581 | EMX1-NGGAAAA-T1 for |
| | GEBx0305 |
| 582 | EMX1-NGGAAAA-T1 for |
| | SpCas9 |
| 583 | EMX1-NGGAAAA-T2 for |
| | GEBx0305 |
| 584 | EMX1-NGGAAAA-T2 for |
| | SpCas9 |
| 585 | EMX1-NGGAAAA-T3 for |
| | GEBx0305 |
| 586 | EMX1-NGGAAAA-T3 for |
| | SpCas9 |
| 587 | EMX1-NGGAAAA-T4 for |
| | GEBx0305 |
| 588 | EMX1-NGGAAAA-T4 for |
| | SpCas9 |
| 589 | EMX1-NGGAAAA-T5 for |
| | GEBx0305 |
| 590 | EMX1-NGGAAAA-T5 for |
| | SpCas9 |
| 591 | POLQ-NGGAAAA-TI for |
| | GEBx0305 |
| 592 | POLQ-NGGAAAA-T1 for |
| | SpCas9 |
| 593 | POLQ-NGGAAAA-T2 for |
| | GEBx0305 |
| 594 | POLQ-NGGAAAA-T2 for |
| | SpCas9 |
| 595 | POLQ-NGGAAAA-T3 for |
| | GEBx0305 |
| 596 | POLQ-NGGAAAA-T3 for |
| | SpCas9 |
| 597 | POLQ-NGGAAAA-T4 for |
| | GEBx0305 |
| 598 | POLQ-NGGAAAA-T4 for |
| | SpCas9 |
| 599 | POLQ-NGGAAAA-T5 for |
| | GEBx0305 |
| 600 | POLQ-NGGAAAA-T5 for |
| | SpCas9 |
| 601 | TTR-NGGAAAA-T1 for |
| | GEBx0305 |
| 602 | TTR-NGGAAAA-TI for |
| | SpCas9 |
| 603 | TTR-NGGAAAA-T2 for |
| | GEBx0305 |
| 604 | TTR-NGGAAAA-T2 for |
| | SpCas9 |
| 605 | TTR-NGGAAAA-T3 for |
| | GEBx0305 |
| 606 | TTR-NGGAAAA-T3 for |
| | SpCas9 |
| 607 | TTR-NGGAAAA-T4 for |
| | GEBx0305 |
| 608 | TTR-NGGAAAA-T4 for |
| | SpCas9 |
| 609 | TTR-NGGAAAA-T5 for |
| | GEBx0305 |
| 610 | TTR-NGGAAAA-T5 for |
| | SpCas9 |
| 611 | CD34-NGGTACT-T1 for |
| | GEBx0308 |
| 612 | CD34-NGGTACT-T1 for |
| | SpCas9 |
| 613 | CD34-NGGTACT-T2 for |
| | GEBx0308 |
| 614 | CD34-NGGTACT-T2 for |
| | SpCas9 |
| 615 | CD34-NGGTACT-T3 for |
| | GEBx0308 |
| 616 | CD34-NGGTACT-T3 for |
| | SpCas9 |
| 617 | CD34-NGGTACT-T4 for |
| | GEBx0308 |
| 618 | CD34-NGGTACT-T4 for |
| | SpCas9 |
| 619 | CFTR-NGGTACT-T1 for |
| | GEBx0308 |
| 620 | CFTR-NGGTACT-T1 for |
| | SpCas9 |
| 621 | CFTR-NGGTACT-T2 for |
| | GEBx0308 |
| 622 | CFTR-NGGTACT-T2 for |
| | SpCas9 |
| 623 | CFTR-NGGTACT-T3 for |
| | GEBx0308 |
| 624 | CFTR-NGGTACT-T3 for |
| | SpCas9 |
| 625 | CFTR-NGGTACT-T4 for |
| | GEBx0308 |
| 626 | CFTR-NGGTACT-T4 for |
| | SpCas9 |
| 627 | CFTR-NGGTACT-T5 for |
| | GEBx0308 |
| 628 | CFTR-NGGTACT-T5 for |
| | SpCas9 |
| 629 | EMX1-NGGTACT-T1 for |
| | GEBx0308 |
| 630 | EMX1-NGGTACT-T1 for |
| | SpCas9 |
| 631 | EMX1-NGGTACT-T2 for |
| | GEBx0308 |
| 632 | EMX1-NGGTACT-T2 for |
| | SpCas9 |
| 633 | EMX1-NGGTACT-T3 for |
| | GEBx0308 |
| 634 | EMX1-NGGTACT-T3 for |
| | SpCas9 |
| 635 | EMX1-NGGTACT-T4 for |
| | GEBx0308 |
| 636 | EMX1-NGGTACT-T4 for |
| | SpCas9 |
| 637 | EMX1-NGGTACT-T5 for |
| | GEBx0308 |
| 638 | EMX1-NGGTACT-T5 for |
| | SpCas9 |
| 639 | POLQ-NGGTACT-T1 for |
| | GEBx0308 |
| 640 | POLQ-NGGTACT-T1 for |
| | SpCas9 |
| 641 | POLQ-NGGTACT-T2 for |
| | GEBx0308 |
| 642 | POLQ-NGGTACT-T2 for |
| | SpCas9 |
| 643 | POLQ-NGGTACT-T3 for |
| | GEBx0308 |
| 644 | POLQ-NGGTACT-T3 for |
| | SpCas9 |
| 645 | POLQ-NGGTACT-T4 for |
| | GEBx0308 |
| 646 | POLQ-NGGTACT-T4 for |
| | SpCas9 |
| 647 | POLQ-NGGTACT-T5 for |
| | GEBx0308 |
| 648 | POLQ-NGGTACT-T5 for |
| | SpCas9 |
| 649 | TTR-NGGTACT-T1 for |
| | GEBx0308 |
| 650 | TTR-NGGTACT-T1 for |
| | SpCas9 |
| 651 | VEGFA-AAAAAA-T1 for |
| | GEBx0305 |
| 652 | VEGFA-AAAAAA-T2 for |
| | GEBx0305 |
| 653 | VEGFA-AAAAAA-T3 for |
| | GEBx0305 |
| 654 | VEGFA-AAAAAA-T4 for |
| | GEBx0305 |
| 655 | RNF2-AAAAAA-T1 for |
| | GEBx0305 |
| 656 | RNF2-AAAAAA-T2 for |
| | GEBx0305 |
| 657 | RNF2-AAAAAA-T3 for |
| | GEBx0305 |
| 658 | RNF2-AAAAAA-T4 for |
| | GEBx0305 |
| 659 | CD34-CATACT-T1 for |
| | GEBx0308 |
| 660 | CD34-CATACT-T2 for |
| | GEBx0308 |
| 661 | CFTR-CATACT-TI for |
| | GEBx0308 |
| 662 | CFTR-CATACT-T2 for |
| | GEBx0308 |
| 663 | CFTR-CATACT-T3 for |
| | GEBx0308 |
| 664 | CFTR-CATACT-T4 for |
| | GEBx0308 |
| 665 | CFTR-CATACT-T5 for |
| | GEBx0308 |
| 666 | RFN2-CATACT-TI for |
| | GEBx0308 |
| 667 | RFN2-CATACT-T2 for |
| | GEBx0308 |
| 668 | RFN2-CATACT-T3 for |
| | GEBx0308 |
| 669 | RFN2-CATACT-T4 for |
| | GEBx0308 |
| 676 | POLQ-CATACT-TI for |
| | GEBx0308 |
| 671 | POLQ-CATACT-T2 for |
| | GEBx0308 |
| 672 | POLQ-CATACT-T3 for |
| | GEBx0308 |
| 673 | POLQ-CATACT-T4 for |
| | GEBx0308 |
| 674 | POLQ-CATACT-T5 for |
| | GEBx0308 |
| 675 | CD34-TATACT-TI for |
| | GEBx0308 |
| 676 | CD34-TATACT-T2 for |
| | GEBx0308 |
| 677 | CD34-TATACT-T4 for |
| | GEBx0308 |
| 678 | CD34-TATACT-T5 for |
| | GEBx0308 |
| 679 | CD34-TATACT-T7 for |
| | GEBx0308 |
| 680 | CFTR-TATACT-T2 for |
| | GEBx0308 |
| 681 | CFTR-TATACT-T5 for |
| | GEBx0308 |
| 682 | DNMT1-TATACT-T1 |
| | for GEBx0308 |
| 683 | POLQ-TATACT-TI for |
| | GEBx0308 |
| 684 | POLQ-TATACT-T2 for |
| | GEBx0308 |
| 685 | POLQ-TATACT-T5 for |
| | GEBx0308 |
| 686 | POLQ-TATACT-T6 for |
| | GEBx0308 |
| 687 | CD34-TATACT-T3 for |
| | GEBx0308 |
| 688 | CD34-TATACT-T6 for |
| | GEBx0308 |
| 689 | CFTR-TATACT-T1 for |
| | GEBx0308 |
| 690 | CFTR-TATACT-T3 for |
| | GEBx0308 |
| 691 | CFTR-TATACT-T4 for |
| | GEBx0308 |
| 692 | POLQ-TATACT-T3 for |
| | GEBx0308 |
| 693 | POLQ-TATACT-T4 for |
| | GEBx0308 |
| 694 | CFTR-NGGAAAA-T3- |
| | 18nt for GEBx0305 |
| 695 | CFTR-NGGAAAA-T3- |
| | 19nt for GEBx0305 |
| 696 | CFTR-NGGAAAA-T3- |
| | 21nt for GEBx0305 |
| 697 | CFTR-NGGAAAA-T3- |
| | 22nt for GEBx0305 |
| 698 | CFTR-NGGAAAA-T3- |
| | 23nt for GEBx0305 |
| 699 | CFTR-NGGAAAA-T3- |
| | 24nt for GEBx0305 |
| 700 | CFTR-NGGAAAA-T3- |
| | 25nt for GEBx0305 |
| 701 | POLQ-NGGAAAA-T5- |
| | 18nt for GEBx0305 |
| 702 | POLQ-NGGAAAA-T5- |
| | 19nt for GEBx0305 |
| 703 | POLQ-NGGAAAA-T5- |
| | 21nt for GEBx0305 |
| 704 | POLQ-NGGAAAA-T5- |
| | 22nt for GEBx0305 |
| 705 | POLQ-NGGAAAA-T5- |
| | 23nt for GEBx0305 |
| 706 | POLQ-NGGAAAA-T5- |
| | 24nt for GEBx0305 |
| 707 | POLQ-NGGAAAA-T5- |
| | 25nt for GEBx0305 |
| 708 | CFTR-NGGTACT-T4- |
| | 18nt for GEBx0308 |
| 709 | CFTR-NGGTACT-T4- |
| | 19nt for GEBx0308 |
| 710 | CFTR-NGGTACT-T4- |
| | 21nt for GEBx0308 |
| 711 | CFTR-NGGTACT-T4- |
| | 22nt for GEBx0308 |
| 712 | CFTR-NGGTACT-T4- |
| | 23nt for GEBx0308 |
| 713 | CFTR-NGGTACT-T4- |
| | 24nt for GEBx0308 |
| 714 | CFTR-NGGTACT-T4- |
| | 25nt for GEBx0308 |
| 715 | CD34-TATACT-T2- |
| | 18nt for GEBx0308 |
| 716 | CD34-TATACT-T2- |
| | 19nt for GEBx0308 |
| 717 | CD34-TATACT-T2- |
| | 21nt for GEBx0308 |
| 718 | CD34-TATACT-T2- |
| | 22nt for GEBx0308 |
| 719 | CD34-TATACT-T2- |
| | 23nt for GEBx0308 |
| 720 | CD34-TATACT-T2- |
| | 24nt for GEBx0308 |
| 721 | CD34-TATACT-T2- |
| | 25nt for GEBx0308 |
| 433 | HumanSpacer3 |
| | for Cas9 |
| 722 | CFTR-NGGAAAA-T1- |
| | 21nt for GEBx0305 |
| 723 | CFTR-NGGAAAA-T5- |
| | 21nt for GEBx0305 |
| 724 | EMX1-NGGAAAA-T5- |
| | 21nt for GEBx0305 |
| 725 | VEGFA-AAAAAA-T1- |
| | 21nt for GEBx0305 |
| 726 | EMX1-NGGTACT-T4- |
| | 21nt for GEBx0308 |
| 727 | POLQ-NGGTACT-T1- |
| | 21nt for GEBx0308 |
| 728 | POLQ-NGGTACT-T2- |
| | 21nt for GEBx0308 |
| 722 | CFTR-NGGAAAA- |
| | T1_21nt |
| 729 | CFTR-NGGAAAA- |
| | T2 21nt |
| 730 | CFTR-NGGAAAA- |
| | T4_21nt |
| 723 | CFTR-NGGAAAA- |
| | T5 21nt |
| 731 | EMX1-NGGAAAA- |
| | T1_21nt |
| 732 | EMX1-NGGAAAA- |
| | T2_21nt |
| 733 | EMX1-NGGAAAA- |
| | T3 21nt |
| 734 | EMX1-NGGAAAA- |
| | T4 21nt |
| 724 | EMX1-NGGAAAA- |
| | T5_21nt |
| 735 | POLQ-AAAAAA- |
| | T1 21nt |
| 736 | POLQ-AAAAAA- |
| | T2 21nt |
| 737 | POLQ-AAAAAA- |
| | T3_21nt |
| 738 | POLQ-AAAAAA- |
| | T4_21nt |
| 739 | POLQ-NGGAAAA- |
| | T1_21nt |
| 740 | POLQ-NGGAAAA- |
| | T2_21nt |
| 741 | POLQ-NGGAAAA- |
| | T3_21nt |
| 742 | POLQ-NGGAAAA- |
| | T4 21nt |
| 655 | RNF2-AAAAAA- |
| | T1 21nt |
| 743 | RNF2-AAAAAA- |
| | T2_21nt |
| 744 | RNF2-AAAAAA- |
| | T3_21nt |
| 658 | RNF2-AAAAAA- |
| | T4 21nt |
| 725 | VEGFA-AAAAAA- |
| | T1 21nt |
| 652 | VEGFA-AAAAAA- |
| | T2 21nt |
| 745 | VEGFA-AAAAAA- |
| | T3 21nt |
| 654 | VEGFA-AAAAAA- |
| | T4 21nt |
| 746 | POLQ-AAAAAA- |
| | T1 20nt |
| 747 | POLQ-AAAAAA- |
| | T2 20nt |
| 748 | POLQ-AAAAAA- |
| | T3 20nt |
| 749 | POLQ-AAAAAA- |
| | T4 20nt |
| 659 | CD34-CATACT- |
| | T1 21nt |
| 750 | CD34-NGGTACT- |
| | T1 21nt |
| 751 | CD34-NGGTACT- |
| | T2 21nt |
| 752 | CD34-NGGTACT- |
| | T3 21nt |
| 753 | CD34-NGGTACT- |
| | T4 21nt |
| 662 | CFTR-CATACT- |
| | T2_21nt |
| 754 | CFTR-CATACT- |
| | T3 21nt |
| 665 | CFTR-CATACT- |
| | T5 21nt |
| 755 | CFTR-NGGTACT- |
| | T1 21nt |
| 756 | CFTR-NGGTACT- |
| | T2 21nt |
| 757 | CFTR-NGGTACT- |
| | T3 21nt |
| 758 | CFTR-NGGTACT- |
| | T5 21nt |
| 759 | EMX1-NGGTACT- |
| | T1_21nt |
| 760 | EMX1-NGGTACT- |
| | T2 21nt |
| 761 | EMX1-NGGTACT- |
| | T3 21nt |
| 726 | EMX1-NGGTACT- |
| | T4_21nt |
| 762 | EMX1-NGGTACT- |
| | T5 21nt |
| 672 | POLQ-CATACT- |
| | T3 21nt |
| 674 | POLQ-CATACT- |
| | T5 21nt |
| 727 | POLQ-NGGTACT- |
| | T1_21nt |
| 728 | POLQ-NGGTACT- |
| | T2 21nt |
| 763 | POLQ-NGGTACT- |
| | T4 21nt |
| 764 | POLQ-NGGTACT- |
| | T5 21nt |
| 667 | RNF2-CATACT- |
| | T2 21nt |
| 668 | RNF2-CATACT- |
| | T3 21nt |
| 765 | CFTR-NGGCCT-T1 |
| 766 | CFTR-NGGCCT-T2 |
| 767 | CFTR-NGGCCT-T3 |
| 768 | CFTR-NGGCCT-T4 |
| 769 | CFTR-NGGCCT-T5 |
| 770 | EMX1-NGGCCT-T1 |
| 771 | EMX1-NGGCCT-T2 |
| 772 | EMX1-NGGCCT-T3 |
| 773 | EMX1-NGGCCT-T4 |
| 774 | EMX1-NGGCCT-T5 |
| 775 | POLQ-NGGCCT-T1 |
| 776 | POLQ-NGGCCT-T2 |
| 777 | POLQ-NGGCCT-T3 |
| 778 | POLQ-NGGCCT-T4 |
| 779 | POLQ-NGGCCT-T5 |
| 780 | POLQ-NGGCCT-T6 |
| 781 | POLQ-NGGCCT-T7 |
| 782 | TTR-NGGCCT-T1 |
| 783 | TTR-NGGCCT-T2 |
| 784 | TTR-NGGCCT-T3 |
| 785 | CFTR-NGGCCT-T5- |
| | 18 nt |
| 786 | CFTR-NGGCCT-T5- |
| | 19 nt |
| 787 | CFTR-NGGCCT-T5- |
| | 21 nt |
| 788 | CFTR-NGGCCT-T5- |
| | 22 nt |
| 789 | CFTR-NGGCCT-T5- |
| | 23 nt |
| 790 | CFTR-NGGCCT-T5- |
| | 24 nt |
| 791 | CFTR-NGGCCT-T5- |
| | 25 nt |
| 792 | TTR-NGGCCT-T1-18 nt |
| 793 | TTR-NGGCCT-T1-19 nt |
| 794 | TTR-NGGCCT-T1-21 nt |
| 795 | TTR-NGGCCT-T1-22 nt |
| 796 | TTR-NGGCCT-T1-23 nt |
| 797 | TTR-NGGCCT-T1-24 nt |
| 798 | TTR-NGGCCT-T1-25 nt |
| 799 | CFTR-NGGCCT-T1- |
| | 24 nt |
| 800 | CFTR-NGGCCT-T2- |
| | 24 nt |
| 801 | CFTR-NGGCCT-T3- |
| | 24 nt |
| 802 | CFTR-NGGCCT-T4- |
| | 24 nt |
| 803 | EMX1-NGGCCT-T1- |
| | 24 nt |
| 804 | EMX1-NGGCCT-T2- |
| | 24 nt |
| 805 | EMX1-NGGCCT-T3- |
| | 24 nt |
| 806 | EMX1-NGGCCT-T4- |
| | 24 nt |
| 807 | EMX1-NGGCCT-T5- |
| | 24 nt |
| 808 | POLQ-NGGCCT-T1- |
| | 24 nt |
| 809 | POLQ-NGGCCT-T2- |
| | 24 nt |
| 810 | POLQ-NGGCCT-T3- |
| | 24 nt |
| 811 | POLQ-NGGCCT-T4- |
| | 24 nt |
| 812 | POLQ-NGGCCT-T5- |
| | 24 nt |
| 813 | POLQ-NGGCCT-T6- |
| | 24 nt |
| 814 | POLQ-NGGCCT-T7- |
| | 24 nt |
| 815 | TTR-NGGCCT-T2-24nt |
| 816 | TTR-NGGCCT-T3-24nt |
| 817 | CFTR-NGGTACC-T1 |
| 818 | CFTR-NGGTACC-T2 |
| 819 | CFTR-NGGTACC-T3 |
| 820 | CFTR-NGGTACC-T4 |
| 821 | CFTR-NGGTACC-T5 |
| 822 | CFTR-NGGTACC-T6 |
| 823 | POLQ-NGGTACC-T1 |
| 824 | POLQ-NGGTACC-T2 |
| 825 | POLQ-NGGTACC-T3 |
| 826 | POLQ-NGGTACC-T4 |
| 827 | POLQ-NGGTACC-T5 |
| 828 | CFTR-TGTACC-T1 |
| 829 | CFTR-TGTACC-T3 |
| 830 | CFTR-TGTACC-T5 |
| 831 | CFTR-TGTACC-T6 |
| 832 | POLQ-TGTACC-T1 |
| 833 | POLQ-TGTACC-T3 |
| 834 | POLQ-TGTACC-T4 |
| 835 | POLQ-TGTACC-T6 |

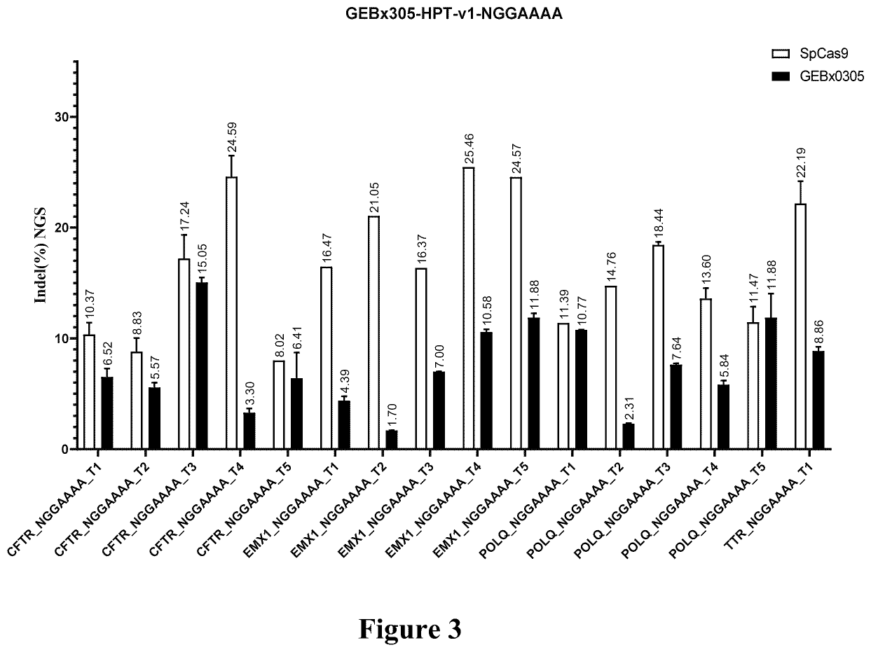

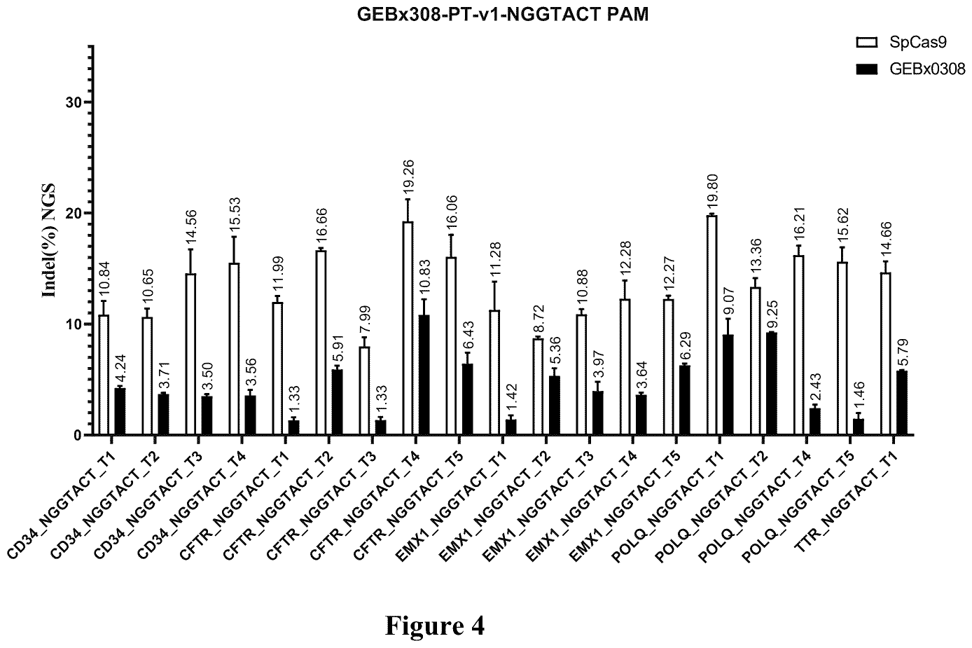

FIG. 3 shows the indel levels of GEBx0305 across 16 targets with GGAAAA-PAM in HEK293T cell line. sgRNA sequence used for GEBx0305 in this experiment harbored GEBx0305-HPT-V1 scaffold (WT, SEQ ID NO. 465) with 20 nt spacer sequences (SEQ ID NOs: 571, 573, 575, 577, 579, 581, 583, 585, 587, 589, 591, 593, 595, 597, 599, and 601). And SpCas9 targeting the corresponding sites (SEQ ID NOs: 572, 574, 576, 578, 580, 582, 584, 586, 588, 590, 592, 594, 596, 598, 600, and 602) is applied as positive control. FIG. 4 shows the indel levels of GEBx0308 across 19 targets with GGTACT-PAM in HTEK293T cell line. sgRNA sequence used for GEBx0308 in this experiment harbored GEBx0308-PT-V1 scaffold (WT, SEQ ID NO: 451) with 20 nt spacer sequences (SEQ ID NOs: 611, 613, 615, 617, 619, 621, 623, 625, 627, 629, 631, 633, 635, 637, 639, 641, 643, 645, 647, and 649). SpCas9 targeting the corresponding sites (SEQ ID NOs: 612, 614, 616, 618, 620, 622, 624, 626, 628, 630, 632, 634, 636, 638, 640, 642, 644, 646, 648, and 650) is applied as positive control.

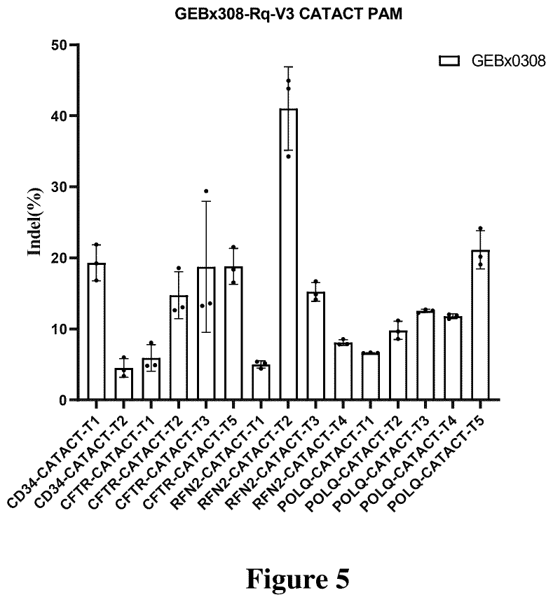

FIG. 5 shows the indel levels of GEBx0308 across 15 targets with CATACT-PAM in HEK293T cell line. psgRNA sequence used for GEBx0308 in this experiment harbored GEBx0308-Rq-V3 scaffold (M0, SEQ ID NO: 454) with 20 nt spacer sequences (SEQ ID NOs: 659-674).

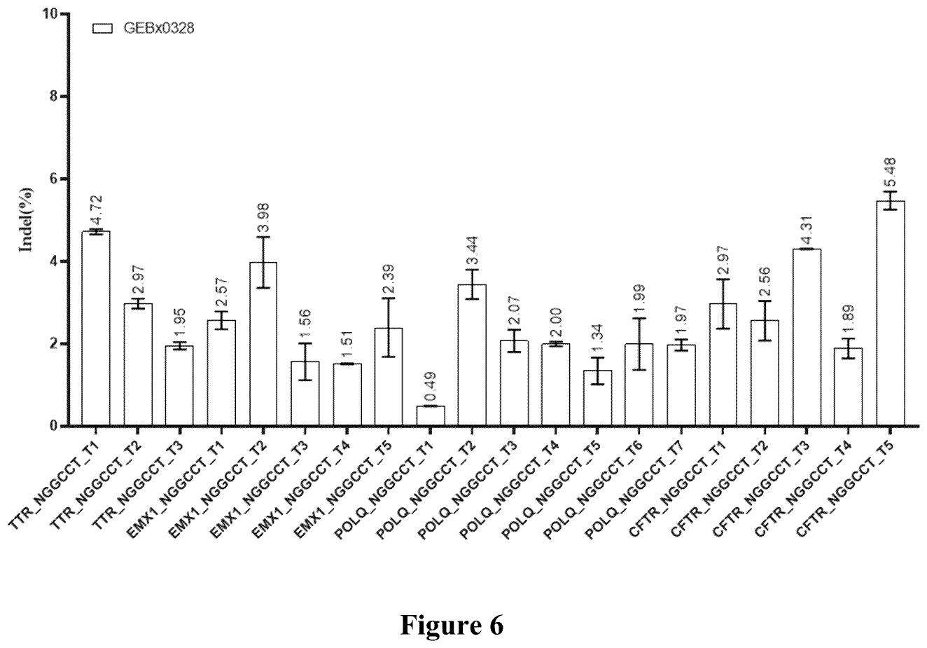

FIG. 6 shows the indel levels of GEBx0328 across 20 targets with NNGCCT-PAM in HEK293T cell line. The sgRNA sequence used for GEBx0328 in this experiment harbored GEBx0328-PT-V1 scaffold (WT, SEQ ID NO: 491) with 20 nt spacer sequences (SEQ ID NOs: 765-784). GEBx0328 shows modest indel activity across 20 targets.

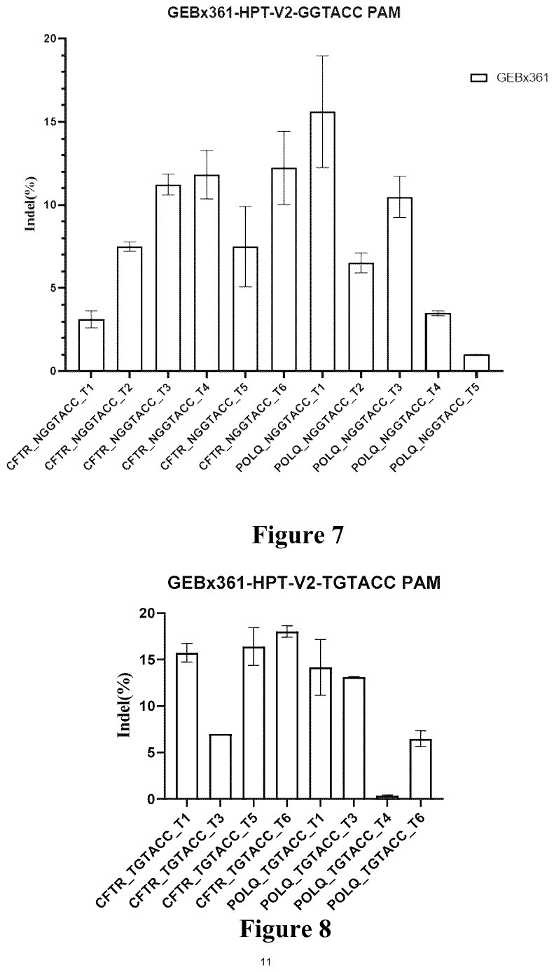

FIG. 7 and FIG. 8 shows the indel levels of GEBx0361 across 19 targets with GGTACC or TGTACC PAM in HEK293T cell line. sgRNA sequence used for GEBx0361 in this experiment harbored GEBx0361-HPT-V2 scaffold (SEQ ID NO: 548) with 20 nt spacer sequences (SEQ ID NOs: 817-835). GEBx0361 shows modest indel activity across 19 targets.

### Example 5: Optimization of the Exemplary CRISPR/Cas System

To further increase Cas protein editing efficiency, variable guide lengths and sgRNA scaffolds were tested. In a set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 250 μL of cells with a density of 50,000 cells/well was seeded onto a 48-well plate 24 hours pre-transfection. Cells were transfected with a lipoplex containing Lipofectamine™ 3000 (0.4 μL/well), P3000 (2 μL/well), pgRNA/pCas protein plasmid (125 ng/well and 375 ng/well, respectively) and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example comprises sequences encoding the corresponding sgRNA scaffold (SEQ ID NO: 465 (GEBx0305), SEQ ID NO: 451 (GEBx0308), or SEQ ID NO: 491 (GEBx0328) and any one of the corresponding spacers (SEQ ID NOs: 575, 599, and 695-707 for GEBx0305; SEQ ID NOs: 625, 676, and 708-721 for GEBx0308 or SEQ ID NOs: 769, 782, and 785-798 for GEBx0328).

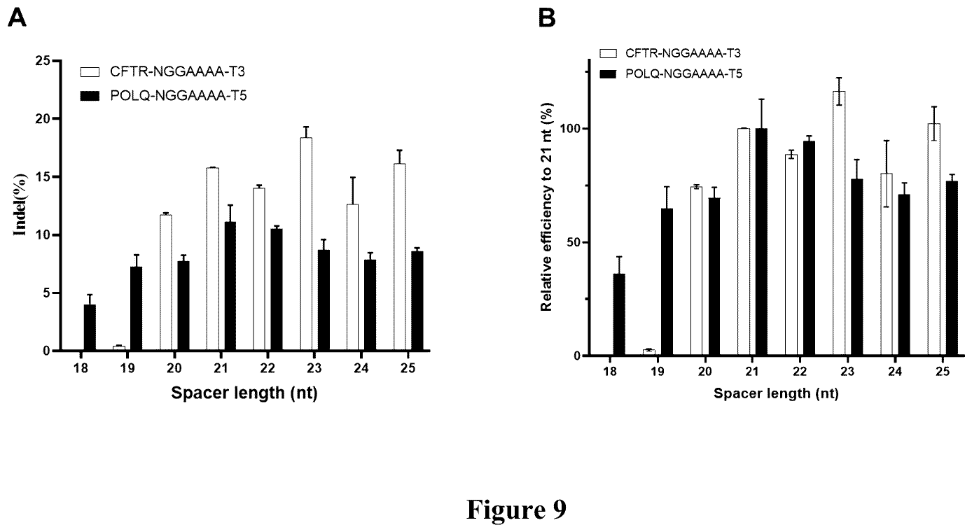

As shown in FIG. 9, indel levels vary depending on the length of the guide sequence of GEBx0305. sgRNA sequence used in this experiment harbored GEBx0305-HPT-V1 scaffold (WT, SEQ ID NO: 465) with CFTR-NGGAAAA-T3 or POLQ-NGGAAAA-T5 spacer sequences ranging from 18 nt to 25 nt (SEQ ID NOs: 575, 599, or 695-707). CFTR-NGGAAAA-T3-23 nt and POLQ-NGGAAAA-T5-25 nt spacer shows the highest indel in each group.

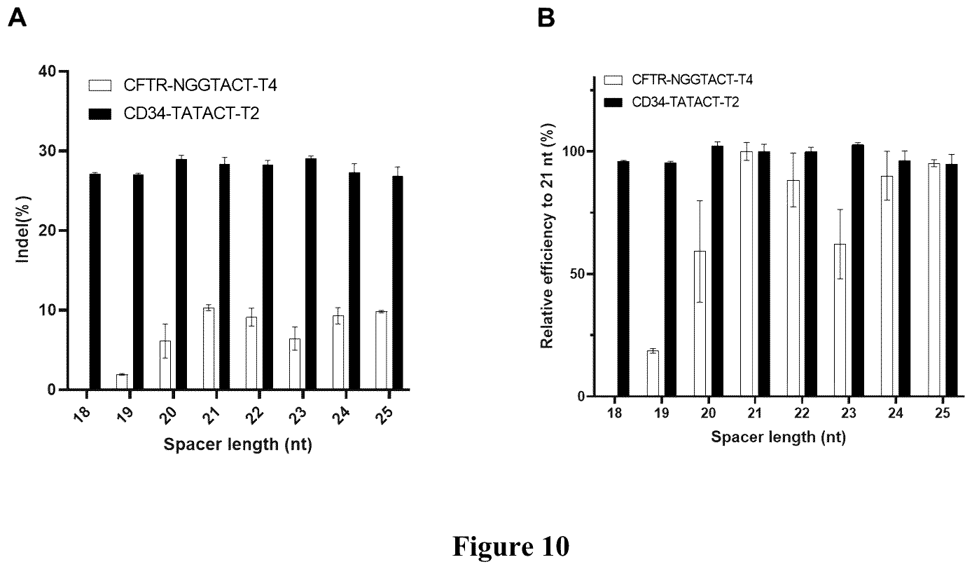

FIG. 10 (A and B) shows indel levels vary depending on the length of the guide sequence of GEBx0308. sgRNA sequence used in this experiment harbored GEBx0308-PT-V1 scaffold (WT, SEQ ID NO: 451) with CFTR-NGGTACT-T4 or CD34-TATACT-T2 spacer sequences ranging from 18 nt to 25 nt (SEQ ID NOs: 625, 676, or 708-721). CFTR-NGGTACT-T4-21 nt spacer shows the highest indel compared with other length spacers.

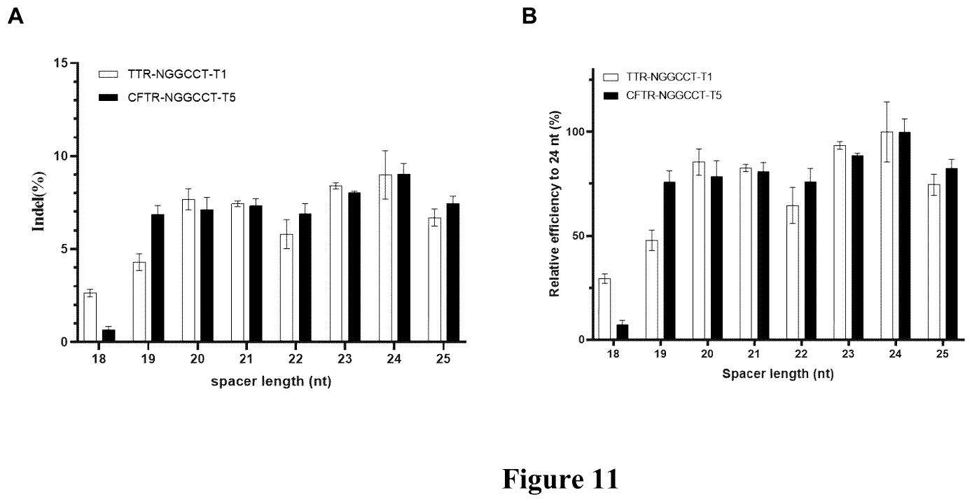

FIG. 11 (A and B) shows indel levels vary depending on the length of the guide sequence of GEBx0328. sgRNA sequence used in this experiment harbored GEBx0328-PT-V1 scaffold (WT, SEQ ID NO: 491) with TTR-NGGCCT-T1 or CFTR-NGGCCT-T5 spacer sequences ranging from 18 nt to 25 nt (SEQ ID NOs: 769, 782, or 785-798). TTR-NGGCCT-T1-24 nt and CFTR-NGGCCT-T5-24 nt spacer shows the highest indel in each group.

In another set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 100 μL of cells with a density of 25,000 cells/well was seeded onto a 96-well plate 24 hours pre-transfection. Cells were transfected with a lipoplex containing Lipofectamine™ 3000 (0.4 μL/well), P3000 (2 μL/well), pCas protein-gRNA plasmid (300 ng/well) and Opti-Mem up to L/well per the manufacturer's protocol. Plated cells were allowed to settle and adhere for 72 hours in a tissue culture incubator at 37° C. and 5% CO₂ atmosphere. The nucleotide sequences of the Cas protein-gRNA used in this example are composed of the Cas CDS, Cas sgRNA scaffold (Table 6) and the corresponding spacers (Table 7).

In another set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 100 μL of cells with a density of 25,000 cells/well was seeded onto a 96-well plate 24 hours pre-transfection. Cells were transfected with a lipoplex containing Lipofectamine™ 3000 (0.4 μL/well), P3000 (2 μL/well), pCas protein-gRNA plasmid (300 ng/well) and Opti-Mem up to L/well per the manufacturer's protocol. Plated cells were allowed to settle and adhere for 72 hours in a tissue culture incubator at 37° C. and 5% CO₂ atmosphere. The nucleotide sequences of the Cas protein-gRNA used in this example are composed of the Cas CDS, Cas sgRNA scaffold and the corresponding spacers.

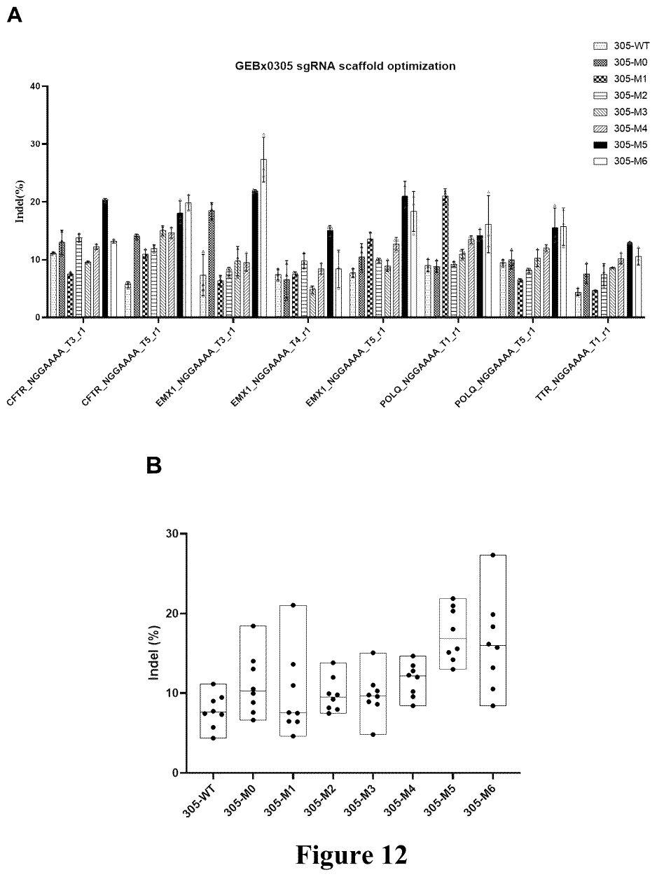

FIG. 12 (A and B) shows indel levels of GEBx0305 targeting endogenous genes with modified RNAs scaffold. sgRNA sequence used in this experiment harbored GEBx0305-HPT-V1 (WT, SEQ ID NO: 465), GEBx0305-Rq-V2 (M0, SEQ ID NO: 466) or GEBx0305-M1 to M6 (SEQ ID NOs: 467-472) scaffold with 20 nt spacers targeting 8 endogenous gene sites (SEQ ID NOs: 575, 579, 585, 587, 589, 591, 599 and 601). GEBx0305-M5 and M6 scaffolds show the top 2 indel mean value.

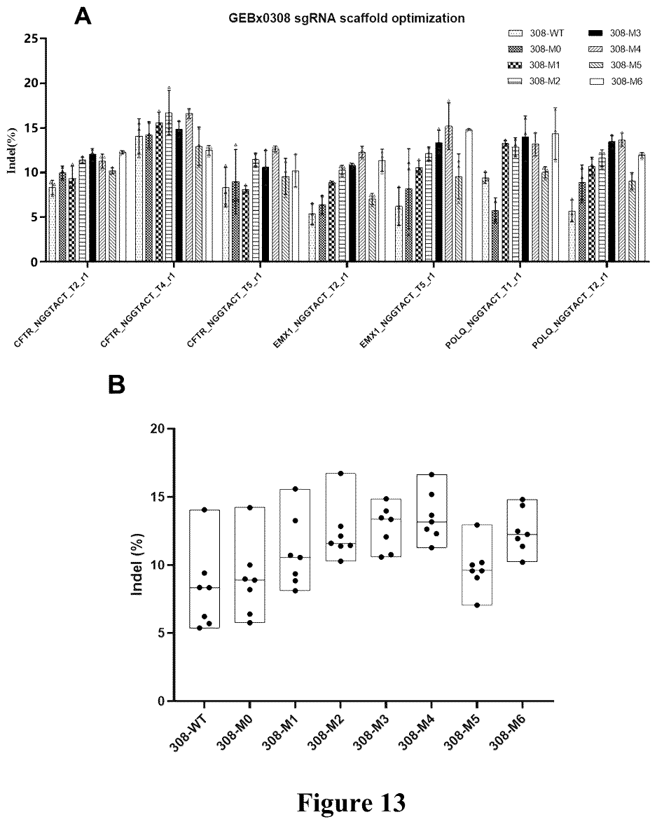

FIG. 13 (A and B) shows indel levels of GEBx0308 targeting endogenous genes with modified RNAs scaffold. sgRNA sequence used in this experiment harbored GEBx0308-PT-V1 (WT, SEQ ID NO: 451), GEBx0308-Rq-V3 (M0, SEQ ID NO: 454) or GEBx0308-M1 to M6 (SEQ ID NOs: 455-460) scaffold with 20 nt spacers targeting 7 endogenous gene sites (SEQ ID NOs: 621, 625, 627, 631, 637, 639, and 641). GEBx0308-M3 and M4 scaffold show the top 2 indel mean value.

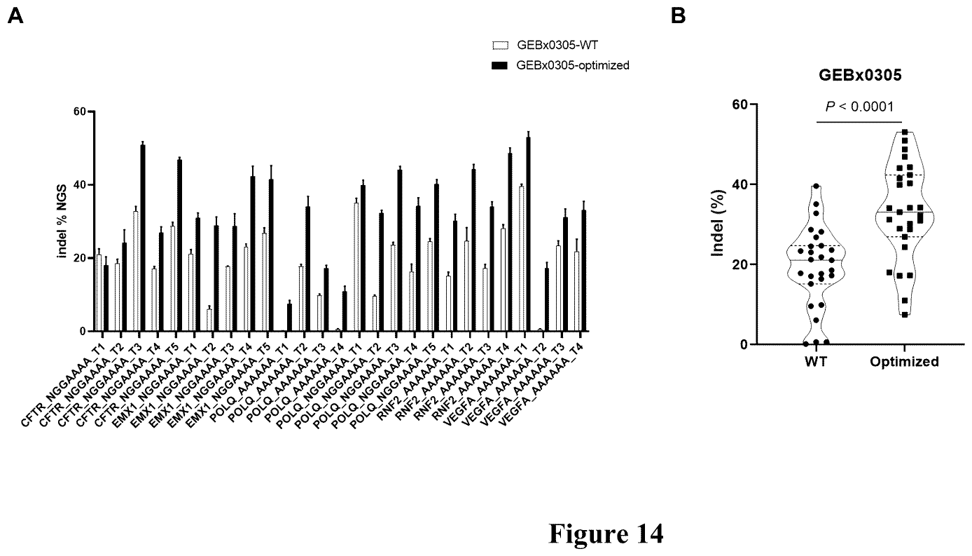

FIG. 14 (A and B) shows the of indel levels of GEBx0305 targeting endogenous genes under optimal conditions. Optimized sgRNA sequence used in this experiment harbored GEBx0305-M5 (SEQ ID NO: 471) scaffold with 21 nt spacers targeting 27 endogenous gene sites (SEQ ID NOs: 652, 654, 655, 658, 696, 703, 722-725, and 729-745). Compared with the WT sgRNA (GEBx0305-HPT-V1 scaffold (SEQ ID NO: 465) with 20 nt spacer (SEQ ID NOs: 571, 573, 575, 577, 579, 581, 583, 585, 587, 589, 591, 593, 595, 597, 599, 651-658, and 746-749), Optimized sgRNA significantly improved the indel levels (P<0.0001) of GEBx0305.

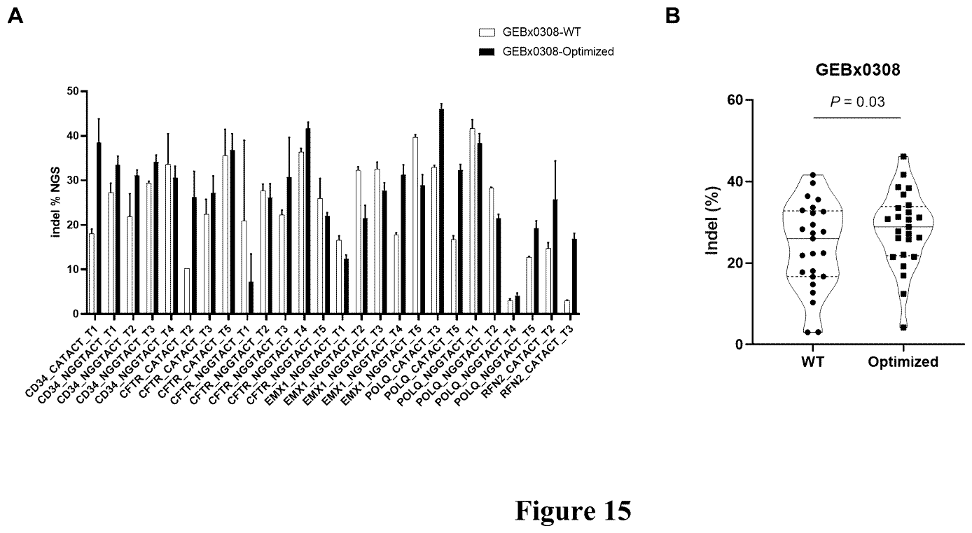

FIG. 15 (A and B) shows the of indel levels of GEBx0308 targeting endogenous genes under optimal conditions. Optimized sgRNA sequence used in this experiment harbored GEBx0308-M4 (SEQ ID NO: 458) scaffold with 21 nt spacers targeting 25 endogenous gene sites (SEQ ID NOs: 710, 659, 665, 667,668, 672, 674, 726, 727, 728, and 750-764). Compared with the WT sgRNA (GEBx0308-PT-V1 scaffold (SEQ ID NO: 451) with 20 nt spacer (SEQ ID NOs: 611, 613, 615, 617, 619, 621, 623, 625, 627, 629, 631, 633, 635, 637, 639, 641, 645, 647, 659, 662, 663, 665, 667, 668, 672, and 674)). Optimized sgRNA slightly improved the indel levels (P=0.03) of GEBx0308.

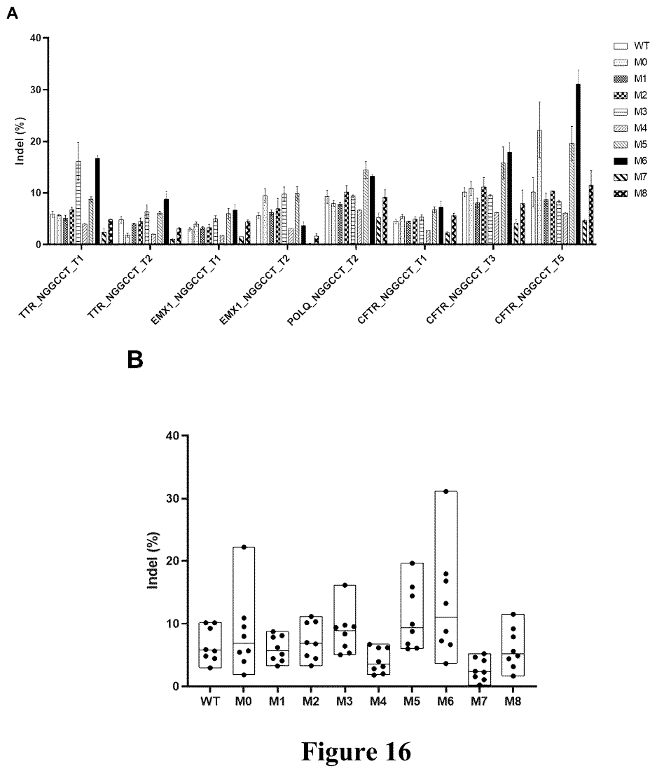

FIG. 16 (A and B) shows indel levels of GEBx0328 targeting endogenous genes with modified RNAs scaffold. sgRNA sequence used in this experiment harbored GEBx0328-PT-V1 (WT, SEQ ID NO: 491), GEBx0328-Rq-V1 (M0, SEQ ID NO: 492) or GEBx0328-M1 to M8 (SEQ ID NOs: 493-500) scaffold with 20 nt spacers targeting 8 endogenous gene sites (SEQ ID NOs: 765, 767, 769, 770, 771, 776, 782, and 783). GEBx0328-M6 scaffolds show the highest indel mean value.

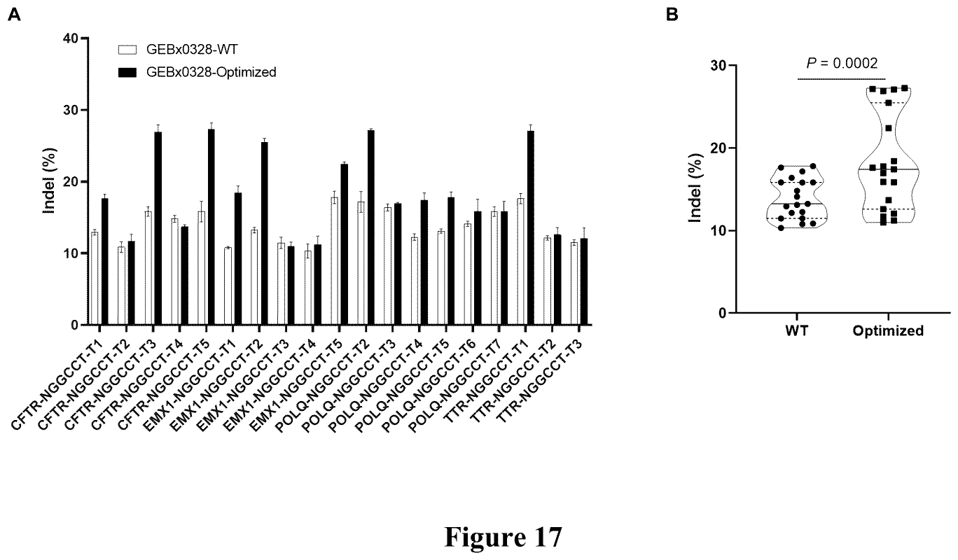

FIG. 17 (A and B) shows the of indel levels of GEBx0328 targeting endogenous genes under optimal conditions. Optimized sgRNA sequence used in this experiment harbored GEBx0328-M6 (SEQ ID NO: 498) scaffold with 24 nt spacers targeting 19 endogenous gene sites (SEQ ID NOs: 790, 797, 799-807, and 809-816). Compared with the WT sgRNA (GEBx0328-PT-V1 scaffold with 20 nt spacer), Optimized sgRNA significantly improved the indel levels (P=0.0002) of GEBx0328.

### Example 6: In Vitro Gene Editing Using RNA

In set of experiments, the HEK293T was cultured in DMEM media supplemented with 10% fetal bovine serum (Gibco™). A volume of 100 μL of cells with a density of 25,000 cells/well were seeded in 96-well plates 24 hours pre-transfection. Cells were transfected with a lipoplex containing Lipofectamine™ RNAiMAX (Invitrogen™) and RNA (20 ng sgRNA, sgRNA: mRNA=1:1-1:8 w/w) and Opti-Mem up to 25 μL/well per the manufacturer's protocol. Plated cells were allowed to settle and adhere for 72 hours in a tissue culture incubator at 37° C. and 5% CO₂ atmosphere. The sgRNA and mRNA sequences are shown in Table 8. The mRNA used in this example is N1-methyl-pseudouridine modified of the uracil.

Primary human liver hepatocytes (PHH) cells were thawed and resuspended in hepatocyte thawing medium with supplements (Lonza, Cat. MCHT50) followed by centrifugation at 100 g for 10 minutes. The supernatant was discarded and the pelleted cells resuspended in hepatocyte plating medium (Lonza, Cat. MP100) plus 10% fetal bovine serum. Cells were counted and plated on Ultra Low Adsorption Cell Culture 96-well plates (Liver Biotech, Cat. LV-ULA002-96W) at a density of 40,000 cells/well. Plated cells were allowed to settle and adhere for 24 hours in a tissue culture incubator at 37° C. and 5% CO₂ atmosphere. After incubation cells were checked for monolayer formation and media was replaced with hepatocyte culture medium (Lonza, Cat. CC-3198) plus 10% fetal bovine serum. Cells were transfected with a lipoplex containing Lipofectamine™ RNAiMAX (Invitrogen™) and RNA (30 ng gRNA, gRNA:mRNA=1:1-1:16 w/w) and Opti-Mem up to 25 μL/well per the manufacturer's protocol. Plated cells were allowed to settle and adhere for 72 hours in a tissue culture incubator at 37° C. and 5% CO₂ atmosphere.

Method of genomic DNA extraction and NGS is identical to that described in Example 4.

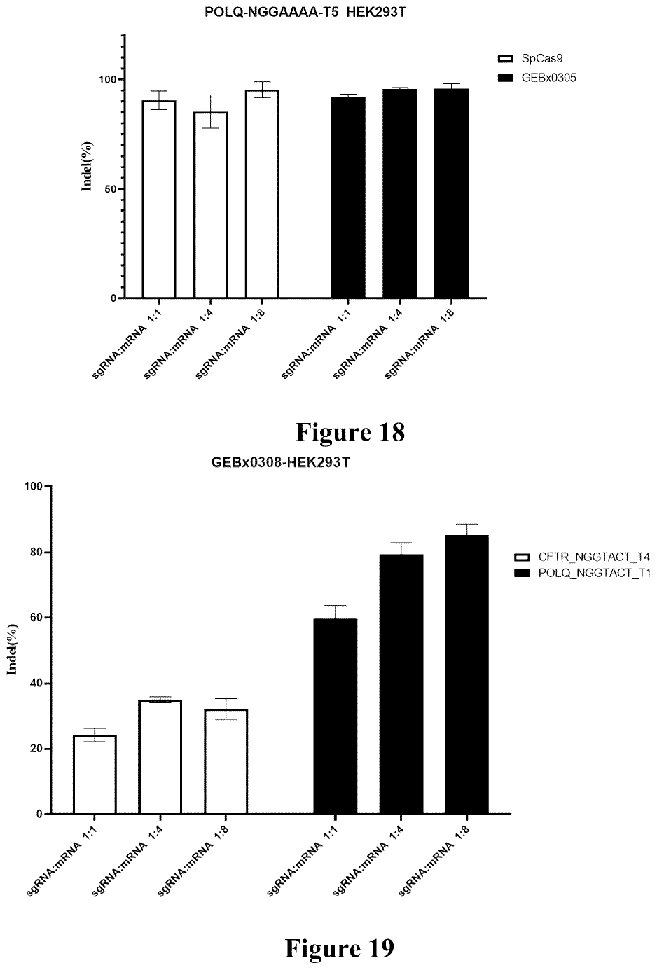

FIG. 18 shows the indel levels of GEBx0305 targeting endogenous genes following transfection of HEK293T cell with lipoplex comprising a fixed amount (20 ng) of sgRNA targeting POLQ-NGGAAAA-T5 site (SEQ ID NO: 855) and different ratios of GEBx0305 mRNA (SEQ ID NO: 851). Equal doses SpCas9 mRNA and sgRNA (SEQ ID NOs: 853 and 854) were used as positive control. GEBx0305 shows an indel efficiency comparable to SpCas9.

FIG. 19 shows the indel levels of GEBx0308 targeting endogenous genes following transfection of HEK293T cell with lipoplex comprising a fixed amount (20 ng) of sgRNA targeting CFTR-NGGTACT-T4 or POLQ-NGGTACT-T1 site (SEQ ID NO: 856/857) and different ratios of GEBx0308 mRNA (SEQ ID NO: 852).

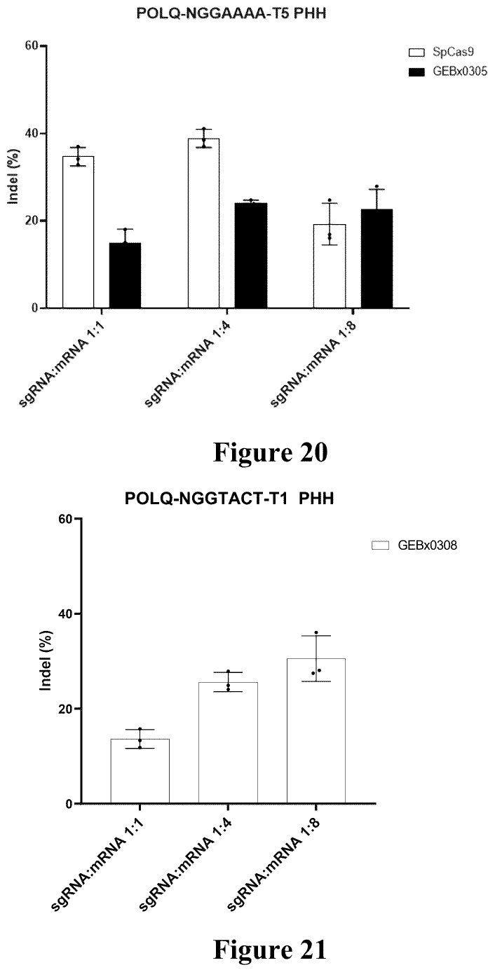

FIG. 20 shows the indel levels of GEBx0305 targeting endogenous genes following transfection of PHH cell with lipoplex comprising a fixed amount (20 ng) of sgRNA targeting POLQ-NGGAAAA-T5 site (SEQ ID NO: 855) and different ratios of GEBx0305 mRNA (SEQ ID NO: 851). Equal doses SpCas9 mRNA and sgRNA (SEQ ID NOs: 853, and 854, Table 9) were used as positive control.

FIG. 21 shows the indel levels of GEBx0308 targeting endogenous genes following transfection of PHH cell with lipoplex comprising a fixed amount (20 ng) of sgRNA targeting POLQ-NGGTACT-T1 site (SEQ ID NO: 857) and different ratios of GEBx0308 mRNA (SEQ ID NO: 852, Table 8).

TABLE 8
The exemplary mRNA and gRNA sequences for gene editing.

| SEQ ID | | |
|---|---|---|
| INOs | name | sequence |
| 851 | GEBx0305 | aGGCUUCCUUUUGUGCAGCUCUUGGGUUUUUUGUGGCUUCCUUCGUUAU |
| | | UGGAGCCAGGCCUACACCCCAGCAACCGCCACCAUGGCCCCCAAGAAGAA |
| | | GAGGAAGGUGGGGCAGAAGGACUUCUACCUCGGGCUGGACAUCGGGACG |
| | | GACAGCGUGGGGUACGCCGUGACGGACGAGCAGUACAACCUGCUCAAGU |
| | | UCUCCGGCGACGCCGCCUGGGGCGUCCACCUCUUCGACGAGGCGGAGCU |
| | | GUCCCAGGAGCGGCGGGCCUUCCGGGUGGCCCGCCGCCGGCUCGACCGGC |
| | | GGAAGCAGCGGGUCAUGCUGCUCCAGGAGCUCUUCGCCCCGGCCAUCCG |
| | | GGCCGUGGACGCCGACUUCUACAAGCGGCUCCAGGCCUCGGACCUCUUC |
| | | CGGGACGAGGCCGGGGACCGCUUCCCGAUCUUCAACGACGUGGACUACA |
| | | CGGACCGGGACUACUACAAGGCGUACCCCACGAUCCACCACCUCAUCUGC |
| | | GACCUCAUGGCGGACGAGACGGCCCACGAUGUCCGGCUCGUGUACCUGG |
| | | CCUGCGCCUGGCUGGUGUCCAACCGGGGGCACUUCCUGUCCAACCUGGA |
| | | CAAGGAGAACAUCUCGGCCCUGAAGAACUUCGACAAGGUGUACUCCGAG |
| | | UUCGAGCACUUCUUCACCGACAACGGGUACGCCUACCCGUGGGCCGAGG |
| | | CCGACCGGCGCACGAUCGGCGAGAUCCUCAAGAAGAAGACCACGGUGAC |
| | | GGCCAAGAACAAGGAGCUGACGCUCGCCUUCACCGGGGGCGGGAUGGCC |
| | | CCCAAGGAGGCGAGCGAGGACUUCCCGUUCGGCCAGCGGGCCAUCAUCA |
| | | AGCUGCUGGCCGGCGGGACGGCCCAGCUCAAGGAGCUCUUCGACCAGGA |
| | | GGCCUACGCCGAGUUCGGCGCGGUCUCCCUCAACUUCGACGACGACAAG |
| | | UUGGAGGAGAUCGCGGGCAACCUGGGCGACGACUUCGCCCUCAUCACCG |
| | | CCCUGCGGCAGGUCUUCGACUGGUCGGUGCUGGUCGAGGCCAUCGGGGA |
| | | CGCCGCCAGCAUCAGCGAGGCCAAGGUGGAGACCUACCGCCAGCACAAG |
| | | GCGGACCUGGCCAUCCUGAAGCGGAUCAUCAAGAACUACCGCCCCGAGA |
| | | AGUACGACGAGGUCUUCCGGGAGAUCGGGAAGGCCAACUACGCGGCCUA |
| | | CGCGUACCACUCCAACAAGAAGGCCGACGCCAAGUUCAAGAAGGUGGAC |
| | | CGCGAGGCCUUCUCGAAGUUCAUCACGGGCAUCGUGAAGGACAUCACGC |
| | | CCAAGGCGGAGGACGAGGAGAUCCUCGCCGACAUGCGGAACCGCCUCGA |
| | | CCUCCGCACCUUCCUGCCCAAGCAGAAGAACACCGACAACCGGGUGAUCC |
| | | CCCACCAGCUCUACCAGUACGAGCUGCACGAGAUCCUCAAGAACGCGGA |
| | | GGGGUACCUCCCGUUCUUGACGGAGAAGGACGAGUCCGGGCUGUCGGUG |
| | | UCCGACAAGAUCCGGAGCAUCUUCGCGUUCAAGAUCCCCUACUUCGUGG |
| | | GGCCCCUGAACGAGAAGAGCGACCACGCGUGGAUCGUCCGCAAGGCGGG |
| | | GAAGAUCUACCCGUGGAACUUCGAGGACAUCGUCGACCUGGACGCGUCC |
| | | GAGGAGGGGUUCAUCCGGCGGAUGACGAACCAGUGCACCUACCUGCCGG |
| | | GCGAGCCCGUCCUCCCGAAGGACAGCCUCCUGUACCACAAGUUCACCGU |
| | | GCUGAACGAGAUCAACAACCUCUCGAUCCACGGUGAGCGGAUCUCGGUG |
| | | GAGCUGAAGCAGCAGAUCUACUCGGAUCUGUUCCUGAACAAGAAGAAGG |
| | | UCACCCGGAAGCGGCUCCGGGAGUACCUGAUCGUGAACGGGGUCGUCGA |
| | | GAAGGACGACACCGACGCGAUCACGGGGAUCGACGAGGAGAUCAAGUCC |
| | | AACCUGCUGCCCCAGAUCUCGUUCCGGCGGCUGCUGGACGAGGGCACGC |
| | | UGUCGGAGGAGGACGUCGAGAAGAUCAUCGAGCGCUCGACGUUCUCCGA |
| | | CGACAAGACGCGCCUGAAGAAGUGGCUGGCGCGGGAGUACCCGCAGCUC |
| | | AACAGCAAGGACGUGGACUACAUCCGGUCCUUGCGGCUGCGGGACUUCG |
| | | GGCGCCUGUCGCAGGCCUUCCUGGAGGGCAUCAUCGCGGCCGACCGGAG |
| | | CACCGGGGAGGCCUUCACCAUCAUCAGCGCGCUCUGGAACACCCAGAAG |
| | | AACCUGAUGGAGCUGCUCUCGGACCAGUUCACCUUCGCCGAGACGAUCG |
| | | AGCGCUUCCGGGAGGACUACUACCGGGAGCACCCGGUGAGCCUCUCGGA |
| | | GCGCAUGGACGAGAUGUACUUGUCCAACUCGGUGAAGCGGAGCGUCUUC |
| | | CGGACGCUCGACAUCGUGAAGGAUGUCAAGAAGGCCUUCGGGGCCCCGA |
| | | AGAAGAUCUUCGUGGAGGUCACCCGGGGCGGGUCGCCCGACCAGAAGGG |
| | | CAAGCGGACCAAGUCCCGGAGCCAGCAGCUGCGGGAGCUGUACGACAAG |
| | | UGCCGGGACGAGGACCUCCCGCUGCUGAAGCAGCAGCUGGAGGCCAUGG |
| | | GGGACGCGGCGGACAGCAAGCUGCAGUCCGAGAAGCUGUUCCUCUACUA |
| | | CCUCCAGCUGGGGAAGUCCGCGUACUCCGACAAGCGGAUCCCGCUGGAG |
| | | GCCCUGGCCUCCAAGGCCUACGACAUCGACCACAUCUACCCGCAGGCCUU |
| | | CGUGAAGGACGACUCCAUCAUCAACAACAAGGUCCUCGUCCUGAGCGAG |
| | | GAGAACGGGGCGAAGAGCAACAACUACCCCAUCCAGGGGUCCAUCCGGC |
| | | ACCACAUGCAGGGCUUCUGGAGCUACCUCCAGGAGGUGGGCCUGAUGUC |
| | | CAAGGAGAAGUACAAGCGCCUCACCCGGAGCACCCCGUUCACGGAGGAG |
| | | GAGCGGUACGGCUUCAUCAACCGCCAGCUGACGAGCACGUCGCAGUCGA |
| | | CGAAGGCGGUGGCCACGCUCCUCAAGGAGCACCUGCCCGAGACCGAGAU |
| | | CGUCUACUGCAAGGCGCGGCUCGUCACGGACUUCCGGCAGGCCUUCGAC |
| | | CUGUUCAAGUCGCGGCAGUACAACGACCUGCACCACGCGGUCGACGCCU |
| | | ACCUGAACAUCGUGACGGGCAACGUGUACAACAUGCGGUUCACCAAGAA |
| | | CUUCAACGUGCACUCGCAGUACUCGAUCAAGACCGAGACGGUCUUCACG |
| | | CGGCCCGUGACGUACAAGUCCCAGACCAUCUGGGACGGCAUCGACAUGC |
| | | UGGCCAAGGUGAAGUCCAUCGCCAACAAGAACACCGCCCACAUCACGAA |
| | | GUACGCGUUCAUGAAGAAGGGCGGGUUCUUCGACCAGCUGCCCGUCCGG |
| | | GCGGCGGACGGGCUCAUCCCCCGGAAGAAGCACCUGCCCACGCAGAAGU |
| | | ACGGCGGGUACAACAAGCCGGGGGUGAUGUUCUUCAUCCCCGUGAAGUA |
| | | CCAGCUGGGGAAGAAGAGCGAGCUGUUCAUCCUCCCCGUGGAGCUGCUC |
| | | UGCGGGAAGCGGUUCCUGGCGGACCCGGAGUUCGCCCAGGCCUACGCCG |
| | | UGAAGCGGCUCGAGCGGAUCCUCAAGAAGCCGGUCGACCAGAUCAGCUU |
| | | CCCGCUCGGCAUGCGGCCCUGGAAGGUCAACACCGUCCUGGCCCUGGAC |
| | | GGCUUCAAGUACUGCAUCACGGGGUCGUCCAACGGGGGCACCCGGCUGA |
| | | UCCUCCAGCCGAUGAUGUCCUUCAGCUCCGCGCCCUACUGGGCGUUCUA |
| | | CCUGAAGAAGCUGGAGGUGUUCGUGGAGAAGAACGCGAACAACCCCCAG |
| | | CACGUCUACGACGAGGCGUACGACAAGGUCAACUGCGAGGACAACCUCA |
| | | AGCUGUACGACCUGUACUGCCAGAAGCUGACGAGCACGAUCUACAGCAA |
| | | GCGGAUCAACAGCCCGCUGGACAUCCUGCUCCAGGGGGGGGAGAAGUUC |
| | | AGCGGGCUGGACAUCAAGCAGCAGGCCCAGGCCCUGCUGAACAUCCACC |
| | | AGGUGUUCGGCAGGAUCGCCGGGGGCUGCGACUUGACGCUCGUCGGCGG |
| | | CAAGAAGGGGUCGGCGGCGACCAGCGCCUCGUCGGCGAUCUCCAACCUG |
| | | GGGAAGCGGUACCAGGACGUGCGGGUCUGGGACACCAGCCCGUCGGGGC |
| | | UCUGGUCCAAGCGGAGCCCCAACCUCCUGGACCUGCUCAAGAGACCCGCC |
| | | GCCACCAAGAAGGCCGGCCAGGCCAAGAAGAAGAAGGACUACAAGGACG |
| | | ACGACGACAAGUGAUCUUCACCCAGUCUGUCUCUUAGAAUUUAUUUCCC |
| | | ACUGACACAAACAAGCCAUUUUGUCUGCUUGUAUAACCCGGAUCCUCAG |
| | | GAAAAAAAAAAAAAAAAAAAAAAAAAAAAAAAAAAAAAAAAAAAAAAA |
| | | AAAAAAAAAAAAAAUGCAUAAAAAAAAAAAAAAAAAAAAAAAAAAAAA |
| | | AAAAAAAAAAAAAAAAAAAAAAAAAAAAAAA |
| 852 | GEBx0308 | aGGCUUCCUUUUGUGCAGCUCUUGGGUUUUUUGUGGCUUCCUUCGUUAU |
| | | UGGAGCCAGGCCUACACCCCAGCAACCGCCACCAUGGCCCCCAAGAAGAA |
| | | GAGGAAGGUGAAGCAGAAGUACUACCUCGGGCUGGACAUGGGGACGUCC |
| | | AGCCUGGGGUGGGCCGUGACCAACGAGAAGUACUCGCUGCUCCGGAAGA |
| | | AGGGGAAGGACCUCUGGGGCGUGCGGCUCUUCGAGGAGGCGAAGACGGC |
| | | CGCCGACCGGCGGUCCUACCGGACCGCCCGGCGGCGGCUGCAGCGGGAG |
| | | AAGGCCCGCAUCGGGUACCUCCGGGAGCUCUUCUCGGAGGAGAUCAACA |
| | | AGAUCGACGCGGGCUUCUUCCAGCGGCUGGAGGACUCCAAGUACCACAA |
| | | GGAGGACAAGCGGGAGCACCAGCCCUUCGCCCUCUUCGCCGACAAGGGC |
| | | UACACGGACCGCGAGUACUUCGAGGACUACCCCACGAUCUUCCACCUGC |
| | | GGAAGGCGCUGCUGGACAUCGAGAACGAGGGGAAGACCUUCGAUGUCCG |
| | | GCUGGUCUACCUGGCCAUCCUGAACAUGUUCAAGCACCGGGGGCACUUC |
| | | CUCAACGCCAACCUGGACGACAAGUCCGGGGGCGACCUCAAGGAGAAGA |
| | | UCCACAUCCUGACGUCCAUGGCGAACAAGCUGUUCGACGACAUCAGCUU |
| | | CAAGACCGUGGACAUCAAGGACAUCGAGCAGAUCCUGGCCAGCAAGGAC |
| | | UACUCGAACUCGACGCGGCUCGAGAAGCUGCUCGAGCUGUUCGAGCUGU |
| | | CGAAGACCAAGAACAAGGUCGAGACGGAGAUCUUCAAGAUGAUCUGCGG |
| | | CCUGAAGGGGACCAUCUCGAAGAUCUUCGCGAACGAGAGCAUGGACGAG |
| | | GAGGCCCAGAAGCUCUCGUUCUCGUUCCGGGACGGGAACUACGACGAGA |
| | | AGAUCGUCGAGAUGGAGGAGGCGCUCUCGGAGGAGUCCUACGAGUUCAU |
| | | CAUGAUCAUGAAGCAGGUCCACGACUGGGCCGUGCUCAGCAACAUCAUG |
| | | AUGGGCGAGGAGUACCUCUCGGUGGCGCGGGUCAAGGCCUACGAGAAGC |
| | | ACAAGAAGGACCUGAACAUGCUCAAGGCCCUGCUCAAGAACUACUCCAU |
| | | CAAGGAGUACAACUCCAUGUUCCGGAUCAUGAAGGACAACAACUACUCG |
| | | UCCUACAUCGGGAGCGUGAUCUCCCAGGAGACGAUCCGGCGCAAGGGGA |
| | | AGUACACGACGGACGACUUCUACAAGAACGUGAAGUCGAUCGUCCUCAA |
| | | GUGCGAGGAGUGCACCGAGAAGGAGUACAUCCUCAGCGAGAUCGAGAAG |
| | | AAGACGUUCCUGCCCAAGCAGAACACGACGUCGAACUCGGUCAUCCCGA |
| | | ACCAGGUCCACAAGAAGGAGCUGAAGCGGAUCCUCGAGAACGCGGCCAA |
| | | GUACCUCGAGUUCCUGAACGACAAGGACGACACGGGGCUCACCGUGUCG |
| | | GAGAAGAUCGUGCGGCUCUUCGAGUUCCAGAUCCCGUACUACGUGGGGC |
| | | CGCUGAGCAACGGCGUCUGCGACGGGAAGAACGUCUGGUCCGUCCGGCG |
| | | CGAGAAGGGCGCCGUGCUGCCGUGGAACUUCGAGGAGAAGAUCGACGUG |
| | | AAGCAGAGCAGCGAGAACUUCAUCACCAAGCUGGUGAACCACUGCACCU |
| | | ACCUCAACGGGGAGACCGUCCUCCCCAAGAACUCGCUGCUCUACGAGAA |
| | | GUUCAUGGUCCUGAACGAGCUGAACAACCUGAAGAUCAACGGGGUGAAG |
| | | AUCGGGGUGGAGCUCAAGCAGAGCAUCUACAACGACGUCUUCAAGAAGG |
| | | GGAAGAAGGUCACGGCCAAGGCCCUGAUCCAGUACCUGAAGAACAUCGG |
| | | GGAGAUCGACGCCACCGAGGAGACGGUGGUGUCGGGGAUCGACGGGGAC |
| | | UUCACGAACCGGCUCGCGAACUACAAGAAGUUCAUCGAGAUCUUCAAGG |
| | | UCCCCACGCUCACCUACGAGCAGGAGCAGAUCGCCGAGGACAUCAUCUU |
| | | CUACUCCACCGUGUACGGGGAGUCGAAGAAGUUCCUCGAGGAGCGGAUC |
| | | AGGGAGAAGUACAACGAGGAGCUGUCGGAGAACCAGAUCAAGCGGAUCC |
| | | UCGGCCUCAAGUUCAAGGACUGGGGCCGGCUCUCGAAGGAGCUGCUGCU |
| | | GCUGCAGGGGAUCGACAAGCAGACCGGCGAGAUCUCCAGCAUCAUCAGC |
| | | AAGAUGUGGUCGGACAACUACAACCUGAUGGAGCUGAUCGCCACCGACA |
| | | AGUUCUCGUACGGGUACAACAUCGAGCAGAAGACCUCGAAGAUCGAGAA |
| | | GGUCCUGUCCGACAUCGAGUACAGCGACCUCGACGAGAUCUACAUCUCG |
| | | GCCCCGGUCAAGCGGAUGGUCUGGCAGACCAUCCUCGUGCUGAAGGAGC |
| | | UCGAGAAGGUCAUGGGCUGCGAGCCCGAGAAGAUCUUCGUCGAGAUGGC |
| | | GCGGGACGCCAACGCGCCGAAGAAGCGGACGGAGUCCCGCAAGAAGAAG |
| | | UUCCUCGAGCUCUACAAGACCUGCAAGAAGGAGUCCCGGGACUGGAAGG |
| | | AGGAGCUCGAGGACACCGAGGAGAGCAAGUUCCGGUCCAAGAAGCUGUA |
| | | CCUGUACUACACCCAGAAGGGGCGGUGCAUGUACAGCGGGGAGGAGAUC |
| | | GAGCUGAAGGAUCUCUUCAACGACAACCUGUACGACAUCGACCACAUCU |
| | | ACCCGCGGCACUUCGUGAAGGACGACAGCAUCGAGAACAACCUCGUGCU |
| | | GGUCCGGAAGGUGGACAACGCCCACAAGUCGGACGCCGUGCUGGAGUCG |
| | | UCGAUCCGCAACAAGAUGUGCGGCUUCUGGAAGAUCCUCCGGGAGGGGA |
| | | GCUUCAUCACCGACGAGAAGUACAAGCGCCUCAUGCGGACCGACGACUU |
| | | CAGCGACGAGGAGCGGGCCGGCUUCAUCAGCCGGCAGAUCGUGGAGACG |
| | | CGGCAGGGGACCAAGGUCAUCGCCGACCUCUUCACGAAGACGUUCCCCG |
| | | AGAGCGAGAUCAUCUACGUGAAGGCGGGGAACGUCUCGGACUUCCGGAA |
| | | GAAGUUCGACUUCCUGAAGUGCCGCAUCGUGAACGACUUCCACCACGCG |
| | | CAGGACGCGUACCUGAACAUCGUGGUGGGGAACGUGUACAACACGAAGU |
| | | UCACGCAGAACCCGAUGAACUUCAUCCGGGAGUACAACAAGGACCCCAA |
| | | GGCCAACAAGUACCACAUGGACAAGCUCUUCGAGUACCCGGUGUCCCGG |
| | | AACGGCCGGGACGCCUGGGUGACGAAGAACGCGGAGUCCAUCUGCAUGG |
| | | UCAAGAAGAUGAUGGAGAAGAACACGCCGCUCAUCACGCGGAUGAACUA |
| | | CGAGAGCCACGGCGGGAUCGCCGACCAGACGAUCUACUCCGCCGAGGAC |
| | | GCCCAGAAGGCCAAGGGCAAGGGGUACAUCAGCAUCAAGUCCUCCGACG |
| | | GCAACCUCCUCGACACCUGCAAGUACGGCGGGAUGAAGAAGUACACCGG |
| | | CACGUACUUCCUCCUGCUGGAGCACACCAAGAAGGAGAAGCGGAUCCGG |
| | | ACCAUCGAGGCCAUGCCGCUCUACCUGAAGGACAAGCUCAACACGCGGG |
| | | AGAAGAUCGAGAAGUACUUCGAGCAGUCCGUGGACUUCGCCUACGAGCA |
| | | GCCGAAGGUGAUCCUGGACAAGAUCAAGAUGUACUCCCUGAUCAAGGUG |
| | | GACGGGUUCUACCUCUACCUGACGGGGCGGACGGGGAACCAGCUCUCCG |
| | | UCUGCAACGCGGUGCAGAUGAGCCUGUCCGCCGGGAUGGUGAAGUACGU |
| | | CCGGAAGAUCUCGAUCGCCAACGAGCGGUACGCCGCCGACAAGGAGUUC |
| | | GAGAAGGACGAGAACAUCUCCGUCCAGAAGAACCUCGAGCUCUACGAGA |
| | | UCCUGAAGGAGAAGCACCUCAACCAGAUCUACGGCAAGCGGCCGAACCC |
| | | CGUCGGCGAGAAGAUCGAGAAGGGGAUCGAGAAGUUCCGGAGCCUCAGC |
| | | GUCCGGAAGCAGAUCUACGUGCUGCUGCAGAUCCUGCAGCUGUCCCAGC |
| | | UGAGCAACCAGGGGGCCGACCUCCUGGAGAUCGGCGGGGAGAAGAAGAC |
| | | CGGGACGUCGAAGCUCAACAAGAACAUCACGGAGAAGCAGGAGUUCAAG |
| | | CUCAUCAACCAGUCCGUGACGGGGCUCUACGAGAACGAGAUCGACCUGC |
| | | UCCGGAUCAAGAGACCCGCCGCCACCAAGAAGGCCGGCCAGGCCAAGAA |
| | | GAAGAAGGACUACAAGGACGACGACGACAAGUGAUCUUCACCCAGUCUG |
| | | UCUCUUAGAAUUUAUUUCCCACUGACACAAACAAGCCAUUUUGUCUGCU |
| | | UGUAUAACCCGGAUCCUCAGGAAAAAAAAAAAAAAAAAAAAAAAAAAA |
| | | AAAAAAAAAAAAAAAAAAAAAAAAAAAAAAAAAAUGCAUAAAAAAAAA |
| | | AAAAAAAAAAAAAAAAAAAAAAAAAAAAAAAAAAAAAAAAAAAAAAAA |
| | | AAA |
| 853 | SpCas9 | AGGUCUAGAGGGAAAUAAGAGAGAAAAGAAGAGUAAGAAGAAAUAUAA |
| | mRNA | GACCCCGGCGCCGCCACCAUGGCCCCUAAGAAAAAGCGGAAGGUGGACA |
| | | AGAAGUACAGCAUCGGCCUGGACAUCGGCACCAAUUCUGUUGGCUGGGC |
| | | CGUGAUCACCGACGAGUACAAGGUGCCCAGCAAGAAAUUCAAGGUGCUG |
| | | GGCAACACCGACCGGCACAGCAUCAAGAAGAAUCUGAUCGGCGCCCUGC |
| | | UGUUCGAUUCUGGCGAAACAGCCGAGGCCACCAGACUGAAGAGAACCGC |
| | | CAGAAGAAGAUACACCCGGCGGAAGAACCGGAUCUGCUACCUGCAAGAG |
| | | AUCUUCAGCAACGAGAUGGCCAAGGUGGACGACAGCUUCUUCCACAGAC |
| | | UGGAAGAGUCCUUCCUGGUGGAAGAGGAUAAGAAGCACGAGCGGCACCC |
| | | CAUCUUCGGCAACAUUGUGGAUGAGGUGGCCUACCACGAGAAGUACCCC |
| | | ACCAUCUACCACCUGAGAAAGAAACUGGUGGACAGCACCGACAAGGCCG |
| | | ACCUGAGACUGAUCUAUCUGGCCCUGGCUCACAUGAUCAAGUUCCGGGG |
| | | CCACUUCCUGAUCGAGGGCGACCUGAAUCCUGACAACAGCGACGUGGAC |
| | | AAGCUGUUCAUCCAGCUGGUGCAGACCUACAACCAGCUGUUCGAGGAAA |
| | | ACCCCAUCAACGCCAGCGGAGUGGAUGCCAAAGCCAUCCUGUCUGCCAG |
| | | ACUGAGCAAGAGCAGACGGCUGGAAAACCUGAUUGCCCAGCUGCCUGGC |
| | | GAGAAGAAGAACGGCCUGUUUGGCAACCUGAUCGCCCUGUCUCUGGGCC |
| | | UGACACCUAACUUCAAGAGCAACUUCGACCUGGCCGAGGACGCCAAACU |
| | | GCAGCUGAGCAAGGACACCUACGACGACGACCUGGAUAACCUGCUGGCC |
| | | CAGAUCGGCGAUCAGUACGCUGAUCUGUUCCUGGCCGCCAAGAACCUGU |
| | | CUGAUGCCAUCCUGCUGAGCGACAUCCUGAGAGUGAACACCGAGAUCAC |
| | | AAAGGCCCCUCUGAGCGCCUCUAUGAUCAAGAGAUACGACGAGCACCAC |
| | | CAGGAUCUGACCCUGCUGAAAGCCCUCGUUAGACAGCAGCUGCCAGAGA |
| | | AGUACAAAGAGAUUUUCUUCGACCAGAGCAAGAACGGCUACGCCGGCUA |
| | | UAUUGAUGGCGGAGCCAGCCAAGAAGAGUUCUACAAGUUCAUCAAGCCC |
| | | AUCCUCGAGAAGAUGGACGGCACCGAGGAACUGCUGGUCAAGCUGAACA |
| | | GAGAGGACCUGCUGCGGAAGCAGAGAACCUUCGACAAUGGCAGCAUCCC |
| | | UCACCAGAUCCACCUGGGAGAACUGCACGCCAUUCUGCGGAGACAAGAG |
| | | GACUUCUACCCAUUCCUGAAGGACAACCGGGAAAAGAUUGAGAAGAUCC |
| | | UGACCUUCAGAAUCCCCUACUACGUGGGACCCCUGGCCAGAGGCAAUAG |
| | | CAGAUUUGCCUGGAUGACCCGGAAGUCCGAGGAAACCAUCACACCCUGG |
| | | AACUUCGAGGAAGUGGUGGAUAAGGGCGCCAGCGCUCAGUCCUUCAUCG |
| | | AGCGGAUGACCAACUUCGAUAAGAACCUGCCUAACGAGAAGGUGCUGCC |
| | | CAAGCACAGCCUGCUGUACGAGUACUUCACCGUGUACAACGAGCUGACC |
| | | AAAGUGAAAUACGUGACCGAGGGAAUGAGAAAGCCCGCCUUUCUGAGCG |
| | | GCGAGCAGAAAAAGGCUAUCGUGGAUCUGCUGUUCAAGACCAACCGGAA |
| | | AGUGACCGUGAAGCAGCUGAAAGAGGACUACUUCAAGAAAAUCGAGUGC |
| | | UUCGACAGCGUGGAAAUCAGCGGCGUGGAAGAUCGGUUUAAUGCCAGCC |
| | | UGGGCACAUACCACGAUCUGCUCAAGAUUAUCAAGGACAAGGACUUCCU |
| | | GGACAACGAAGAGAACGAGGACAUCCUGGAAGAUAUCGUGCUGACCCUG |
| | | ACACUGUUUGAGGACAGAGAGAUGAUCGAGGAACGGCUGAAAACAUACG |
| | | CCCACCUGUUCGACGACAAAGUGAUGAAGCAACUGAAGCGGCGGAGAUA |
| | | CACAGGCUGGGGCAGACUGUCCAGAAAGCUGAUCAACGGCAUCCGGGAC |
| | | AAGCAGUCCGGCAAGACCAUCCUGGACUUUCUGAAGUCCGACGGCUUCG |
| | | CCAAUCGGAACUUCAUGCAGCUGAUUCACGACGACAGCCUCACCUUCAA |
| | | AGAGGAUAUCCAGAAGGCCCAGGUUUCCGGCCAGGGCGAUUCUCUGCAC |
| | | GAGCACAUUGCCAAUCUGGCCGGCUCUCCUGCCAUUAAGAAGGGCAUCC |
| | | UGCAGACAGUGAAGGUGGUGGACGAACUCGUGAAAGUGAUGGGCAGAC |
| | | ACAAGCCCGAGAACAUCGUGAUCGAAAUGGCCAGAGAGAACCAGACCAC |
| | | ACAGAAGGGCCAGAAAAACAGCCGCGAGAGAAUGAAGCGGAUCGAAGAG |
| | | GGCAUCAAAGAGCUGGGCAGCCAGAUCCUGAAAGAACACCCCGUGGAAA |
| | | ACACCCAGCUGCAGAACGAGAAGCUGUACCUGUACUACCUGCAGAAUGG |
| | | ACGGGAUAUGUACGUGGACCAAGAGCUGGACAUCAACCGGCUGUCCGAC |
| | | UACGAUGUGGACCAUAUCGUGCCCCAGUCUUUUCUGAAGGACGACUCCA |
| | | UCGACAACAAGGUCCUGACCAGAAGCGACAAGAACCGGGGCAAGAGCGA |
| | | UAACGUGCCAAGCGAAGAGGUCGUGAAGAAGAUGAAGAACUACUGGCGA |
| | | CAGCUGCUGAACGCCAAGCUGAUUACCCAGCGGAAGUUCGACAACCUGA |
| | | CCAAGGCAGAGAGAGGCGGCCUGUCUGAACUGGAUAAGGCCGGCUUCAU |
| | | CAAGAGACAGCUGGUGGAAACCCGGCAGAUCACCAAGCACGUGGCACAG |
| | | AUUCUGGACUCCCGGAUGAACACUAAGUACGACGAGAACGACAAACUGA |
| | | UCCGGGAAGUGAAAGUCAUCACCCUGAAGUCCAAGCUGGUGUCCGAUUU |
| | | CCGGAAGGAUUUCCAGUUUUACAAAGUGCGCGAGAUCAACAACUACCAU |
| | | CACGCCCACGACGCCUACCUGAAUGCCGUUGUGGGAACAGCCCUGAUCA |
| | | AGAAGUAUCCCAAGCUGGAAAGCGAGUUCGUGUACGGCGACUACAAGGU |
| | | GUACGACGUGCGGAAGAUGAUCGCCAAGAGCGAACAAGAGAUCGGCAAG |
| | | GCCACCGCCAAGUACUUUUUCUACAGCAACAUCAUGAACUUUUUCAAGA |
| | | CCGAGAUUACCCUGGCCAACGGCGAGAUCAGAAAGCGGCCUCUGAUCGA |
| | | GACAAACGGCGAAACCGGCGAGAUUGUGUGGGACAAGGGCAGAGAUUUC |
| | | GCCACCGUGCGGAAAGUGCUGAGCAUGCCCCAAGUGAAUAUCGUGAAAA |
| | | AGACCGAGGUGCAGACAGGCGGCUUCUCCAAAGAGAGCAUCCUGCCUAA |
| | | GCGGAACAGCGACAAGCUGAUCGCCAGAAAGAAGGACUGGGACCCUAAG |
| | | AAGUACGGCGGCUUCGAUAGCCCUACAGUGGCCUAUUCUGUGCUGGUGG |
| | | UGGCCAAAGUGGAAAAGGGCAAGUCCAAGAAGCUCAAGAGCGUCAAAGA |
| | | ACUGCUCGGCAUCACCAUCAUGGAACGGUCCAGCUUCGAGAAGAAUCCC |
| | | AUCGACUUUCUGGAAGCCAAGGGCUACAAAGAAGUCAAGAAGGACCUGA |
| | | UCAUCAAGCUCCCCAAGUACUCCCUGUUCGAGCUGGAAAAUGGCCGGAA |
| | | GAGAAUGCUGGCCUCUGCCGGCGAACUGCAGAAAGGCAAUGAACUGGCC |
| | | CUGCCUAGCAAAUACGUCAACUUCCUGUACCUGGCCAGCCACUAUGAGA |
| | | AGCUGAAGGGCAGCCCCGAGGACAAUGAGCAGAAACAACUGUUCGUGGA |
| | | ACAGCACAAACACUACCUGGACGAGAUCAUCGAGCAGAUCUCCGAGUUC |
| | | AGCAAGAGAGUGAUCCUGGCCGACGCCAACCUGGACAAAGUGCUGUCUG |
| | | CCUACAACAAGCACCGGGAUAAGCCCAUCAGAGAGCAGGCCGAGAAUAU |
| | | CAUCCACCUCUUCACCCUGACCAACCUGGGAGCCCCUGCCGCCUUUAAGU |
| | | ACUUCGACACCACCAUCGACCGGAAGCGGUACACCUCUACCAAAGAGGU |
| | | GCUGGACGCCACACUGAUCCACCAGAGCAUCACCGGCCUGUACGAGACA |
| | | AGAAUCGAUCUGUCCCAGCUCGGCGGCGACAAAAGACCUGCCGCAACAA |
| | | AGAAAGCCGGACAGGCCAAGAAAAAGAAGUGAUAAUAGCGGCCGCGGCC |
| | | CGAUCCAGUGGGCAGGCCAAGGCCUGCUGGGCCCCCGCGGACCCAGGUG |
| | | CUCUGGGUCACGGUCCCUGUCCCCGCGCCCCCGCUUCUGUCUGCCCCAUU |
| | | GUGGCUCCUCAGGCUCUCUCCCCUGCUCUCCCACCUCUACCUCCACCCCC |
| | | ACGGAUCCUCAGGAAAAAAAAAAAAAAAAAAAAAAAAAAAAAAAAAAA |
| | | AAAAAAAAAAAAAAAAAAAAAAAAAAUGCAUAAAAAAAAAAAAAAAAA |
| | | AAAAAAAAAAAAAAAAAAAAAAAAAAAAAAAAAAAAAAAAAAA |
| 854 | SpCas9- | mA*mU*mC*AGCCGGGAGGGUUAGCAGUUUUAGAGCUAGAAAUAGCAAG |
| | POLQ- | UUAAAAUAAGGCUAGUCCGUUAUCAACUUGAAAAAGUGGCACCGAGUCG |
| | NGGAA | GUGCU*mU*mU*mU |
| | AA-T5- | |
| | MS | |
| 855 | GEBx030 | mU*mC*mA*GCCGGGAGGGUUAGCAUGUUUGAGAGUAAUGUAAUGAAAA |
| | 5-POLQ- | UUACGUUACGCGUUCAAAUAAAGGUAAACCCAAAUCGUUGGCUUGACCA |
| | NGGAA | ACUGCACAGUGUGUGCGUUCAGGACUGCUUCAGUGCAGUCCU*mU*mU* |
| | AA-T5- | mU |
| | MS | |
| 856 | GEBx030 | mU*mC*mA*CUGCAAGGCACUCUCUAGUUUGAGAACCUUGUGAAAACAA |
| | 8-CFTR- NGGTAC | GUGUGGAUA*mA*mA*mU |
| | T-T4-MS | GGUGAGUUCAAAUAAGGAUUUAUCCGAAAUCGCCGAAAUGGCCCACAUU |
| 857 | GEBx030 | GUGUGGAUA*mA*mA*mU |
| | 8-POLQ- NGGTAC | A*mG*mG*mGUCUGUAAGGAGUACUGGUUUGAGAACCUUGUGAAAACAA |
| | T-T1-MS | GGUGAGUUCAAAUAAGGAUUUAUCCGAAAUCGCCGAAAUGGCCCACAUU |

### Example 7. Off-Target Profiling in Cell Lines Using GUIDE-Seq

GUIDE-Seq leverages a dsODN to insert into the double-strand break site generated by CRISPR/Cas. The HEK293T was cultured in advanced DMVEM media supplemented with 500 fetal bovine serum (Gibco™). Cells were seeded at a density of 100,000 cells/well in 24-well plate 24 hours prior to transfection. Cells were transfected with 400 ng of pCas protein plasmid, 150 ng of pgRNA plasmid, and 2.5 pmol of dsODN using Lipofectamine 3000 (Invitrogen™) per the manufacturer's protocol, cultured at 37° C. and 5% CO₂, and harvested on day three post-transfection.

For GUIDE-Seq library construction, an amount of 500 ng genomic DNA was used for GUIDE-Seq library construction. Briefly, DNA was fragmented by KAPA Frag Kit (KAPA Biosystems), followed by adaptor ligation and two rounds of hemi-nested PCR enrichment for dsODN-integrated fragments. Final sequencing libraries were quantified by KAPA Library Quantification Kits and sequenced on an Illumina NextSeq 1000 System. Data demultiplexing of Index 1 was performed by bcl2fq (version 2.19), followed by custom scripts for Index 2 demultiplexing, adaptor trimming using the BBduk tool, and analyzed by the GUIDE-seq software. Briefly, unique molecular index (UMI)-tagged FASTQ file was consolidated to generate UMI-consensus sequence, and aligned to human reference genome (hg19) using BWA MEM. High-quality alignments (MAPQ≥50) were used for identifying genomic loci harboring the dsODN as the potential off-target sites. Candidate loci with up to 6 mismatches against the corresponding on-target protospacer were identified as bona fide off-target sites.

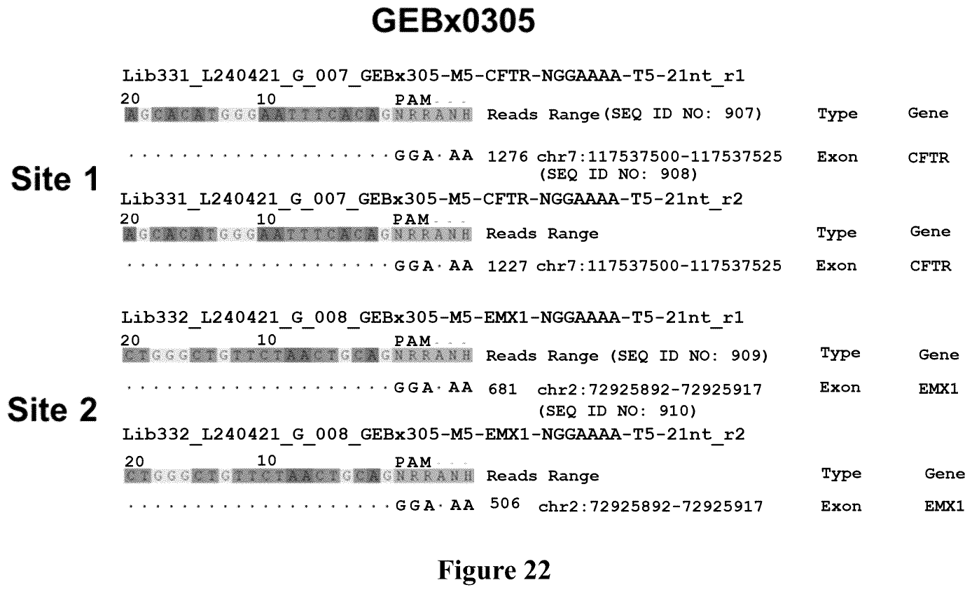

FIG. 22 shows the summary of top Guide-seq insertion sites of GEBx0305. No detectable off-targets could be detected at site 1 (CFTR-NGGAAAA-T5) and site 2 (EMX1-NGGAAAA-T5).

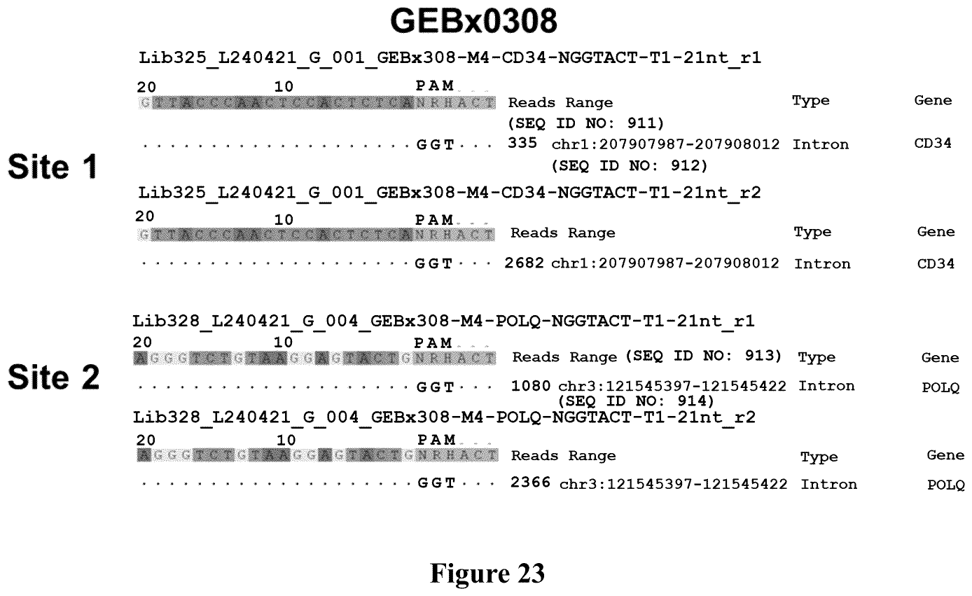

FIG. 23 shows the summary of top Guide-seq insertion sites of GEBx0308. no off-targets could be detected at site 1 (CD34-NGGTACT-T4) and site 2 (POLQ-NGGTACT-T1).

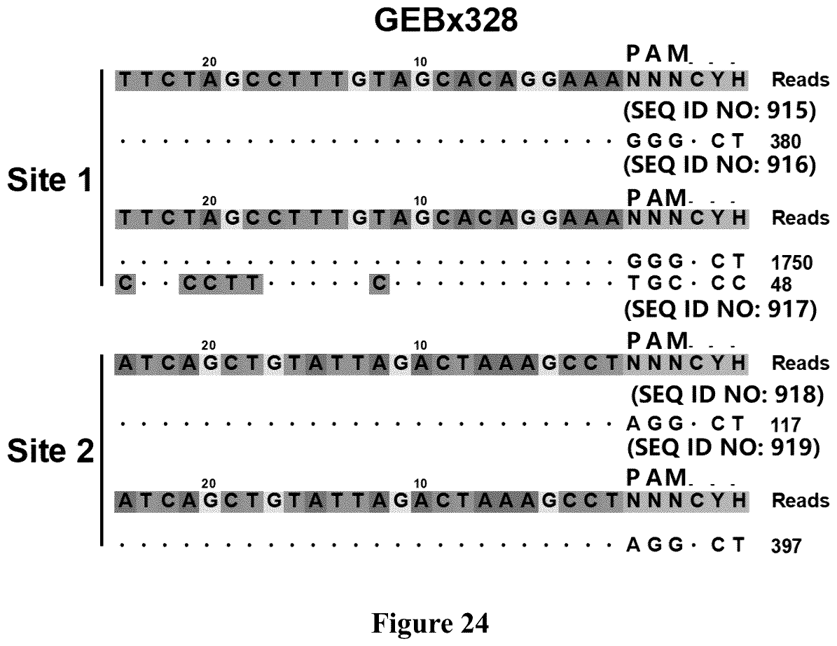

FIG. 24 shows the summary of top Guide-seq insertion sites of GEBx0328. No detectable off-targets could be detected at site 2 (CFTR-NGGCCT-T5) and only one off-target site could be detected at site 1 (CFTR-NGGCCT-T3).

### Example 8. Base-Editing Efficiency Assay for nCas Protein

To generate Cas base editor, Escherichia coli tRNA adenosine deaminase (TadA-8e) was fused to the N-terminal of GEBx0305, GEBx0308, GEB328 nickase (GEBx0305-D11A, GEBx0308-D10A, GEB328-D12A) with an ABE-linker (SEQ ID NO: 868). the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 100 μL of cells with a density of 25,000 cells/well was seeded onto a 96-well plate 24 hours pre-transfection. Cells were transfected with a lipoplex containing Lipofectamine™ 3000 (0.4 μL/well), P3000 (2 μL/well), pABE-gRNA plasmid (300 ng/well) and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pABE/gRNA plasmids used in this example comprise the Cas-ABE CDS (SEQ ID NOs: 861, 862, and 863, Table 9), Cas sgRNA scaffold and the corresponding spacers. 72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples were subjected to amplicons NGS analyses.

To quantitatively determine the efficiency of editing at the target location in the genome, NGS was utilized to identify the presence of insertions and deletions introduced by gene editing. Primers used for NGS which around the target area within the endogenous genes were designed. Additional PCR was performed per the manufacturer's protocols (Illumina) to add chemistry for sequencing. The amplicons were sequenced on Illumina iSeq 100. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an A to G substitution was calculated. The number of the reads mapped the reference genome is more than 1000.

TABLE 9
CDS sequences of the exemplary ABE sequences.

| Name | SEQ ID NOs |
|---|---|
| GEBx0305-ABE-DNA | 861 |
| GEBx0308-ABE-DNA | 862 |
| GEBx0328-ABE(D12A)-DNA | 863 |
| GEBx0305-ABE(D11A)-protein | 864 |
| GEBx0308-ABE(D10A)-protein | 865 |
| GEBx0328-ABE8e-protein | 866 |
| TadA-8e-protein | 867 |
| ABE-linker | 868 |
| nGEBx0305 (D11A)-protein | 869 |
| nGEBx0305 (H859A)-protein | 870 |
| GEBx0305-dCas (D11A/H859A)-protein | 871 |
| nGEBx0308 (D10A)-protein | 872 |
| nGEBx0308 (H862A)-protein | 873 |
| dGEBx0308 (D10A/H862A)-protein | 874 |
| nGEBx0328 (D12A)-protein | 875 |
| nGEBx0328 (H903A)-protein | 876 |
| dGEBx0328 (D12A/H903A)-protein | 877 |

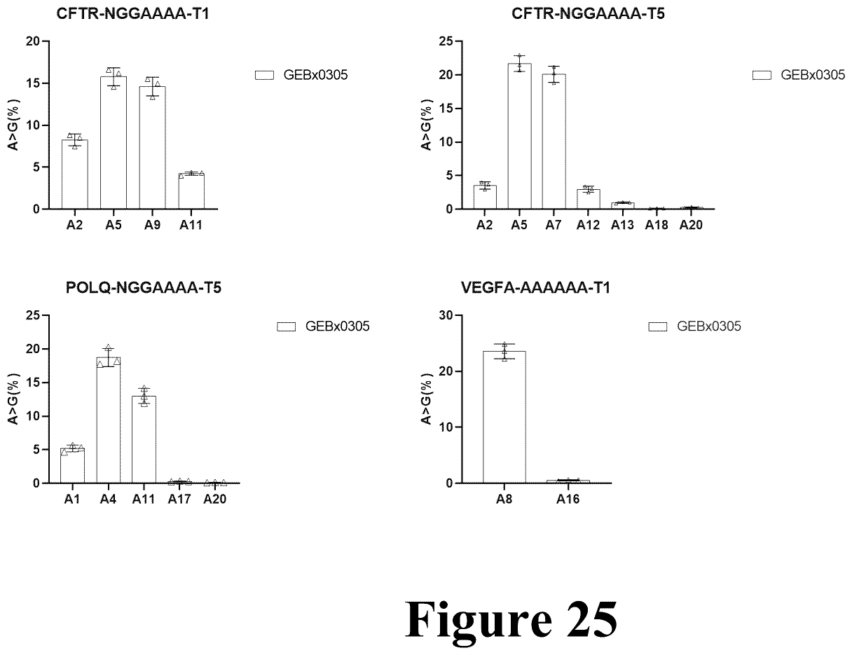

FIG. 25 shows the A to G conversion base editing frequency in HEK293T cells by GEBx0305-ABE at adenines for five endogenous genes sites. sgRNA sequence used in this experiment harbored GEBx0305-M5 (SEQ ID NO: 471) scaffold with 21 nt spacers (SEQ ID NOs: 703, 722, 723, and 725). GEBx0305-ABE exhibited efficient A to G conversion across those sites.

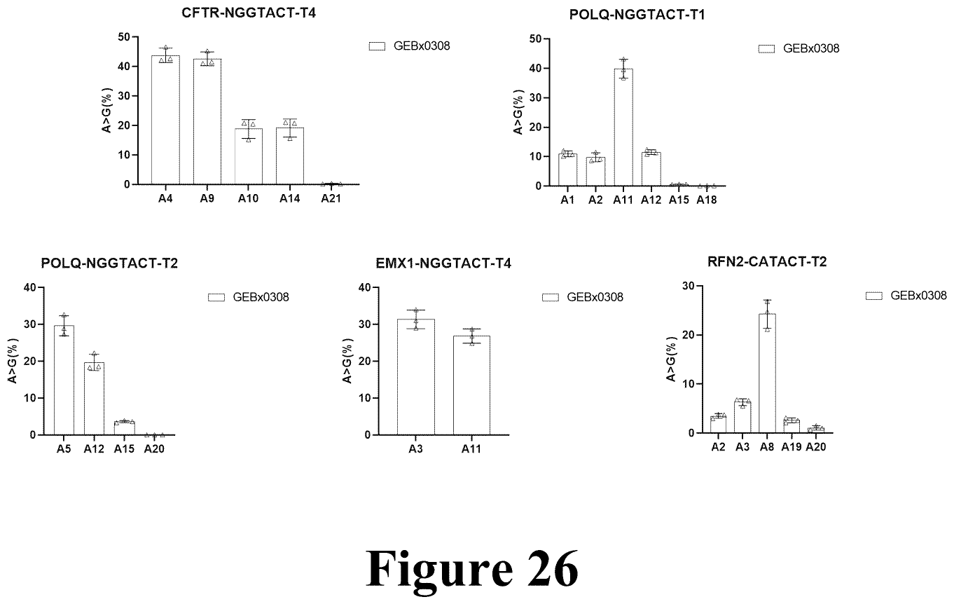

FIG. 26 shows the A to G conversion base editing frequency in HEK293T cells by GEBx0308-ABE at adenines for five endogenous genes sites. sgRNA sequence used in this experiment harbored GEBx0308-M4 (SEQ ID NO: 458) scaffold with 21 nt spacers (SEQ ID NOs: 710, 726, 727, 728, and 667). GEBx0308-ABE exhibited efficient A to G conversion across those sites.

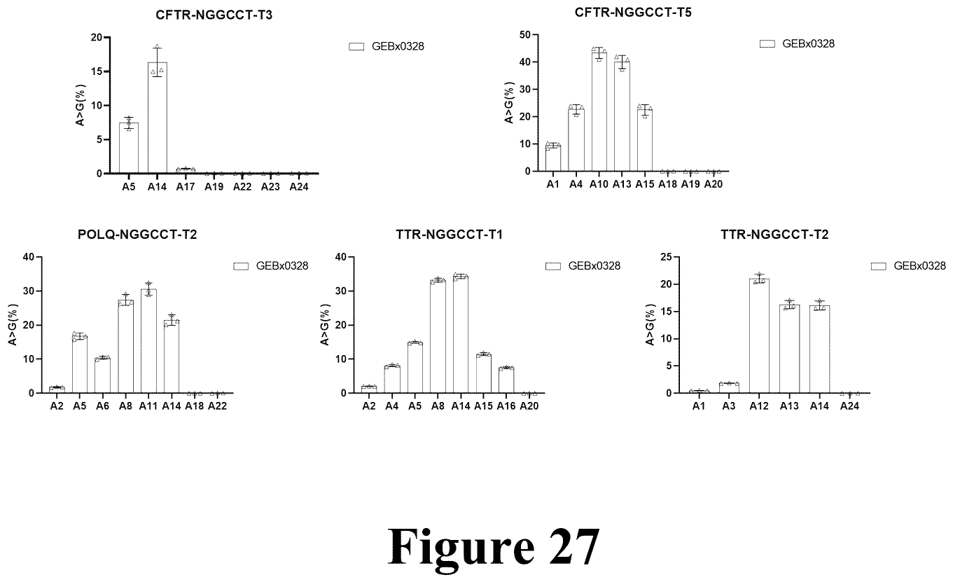

FIG. 27 shows the A to G conversion base editing frequency in HEK293T cells by GEBx0328-ABE at adenines for five endogenous genes sites. sgRNA sequence used in this experiment harbored GEBx0328-M6 (SEQ ID NO: 498) scaffold with 24 nt spacers (SEQ ID NOs: 790, 797, 801, 809, and 815). GEBx0328-ABE exhibited efficient A to G conversion across these sites.

### Example 8. Evaluation of GEBx0308 on Specific Knock Out at RHO-P23H Site in HEK293T

Design of editor plasmid and target plasmid: The editor plasmids were designed and generated containing both SaCas9 (SEQ ID NO: 906) or GEBx0308-coding gene (SEQ ID NO: 252) and the corresponding guide sequences (RHO-P23H sgRNA (SaCas9) which is the single guide RNA for SaCas9 and has the sequence of SEQ ID NO: 904; and RHO-P23H sgRNA which is the single guide RNA for GEBx0308 and has the sequence of SEQ ID NO: 902). The target plasmid was designed and generated harboring sequences of RHO-WT (˜170 bp), RHO-P23H (˜170 bp, c.68C>A) and a 500 bp long unrelated sequence to separate RHO-WT and RHO-P23H.

Evaluation of GEBx0308 on specific knock out at RHO-P23H site in HEK293T, HEK293T cells were cultured in Dulbecco's Modified Eagle's Medium (DMEM, CORNING) containing 2 mM L-glutamine (GlutaMAX™-1, Gibco), supplemented with 10% fetal bovine serum (FETAL BOVINE SERUM, GEMINI) at 37° C. in 5% CO₂-buffered incubators.

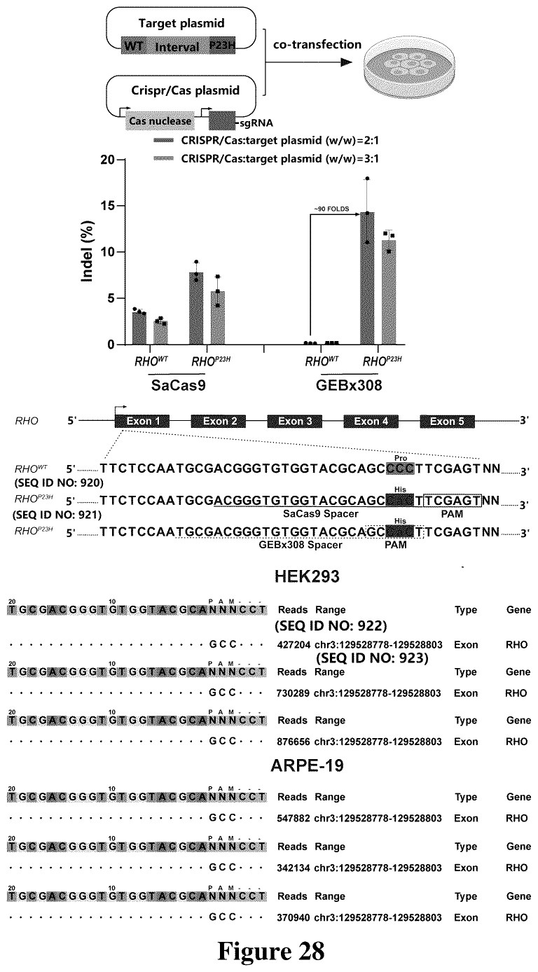

To evaluate the specific knock out efficiency of SaCas9 (SEQ ID NO: 906, Table 10) and GEBx0308 (SEQ ID NO: 252) on RHO-P23H site, Editor and Target Plasmids were co-transfected into HEK293T cells. Each well of a 24-well plate was seeded with 1.0×10⁵ cells. Editor Plasmids and Target plasmids were co-transfected into HEK293T at the ratio of 2:1 and 3:1 (w/w) using Lipofectamine 3000 Reagent as per the manufacturer's instructions (Life Technologies). After 72 hours of incubation, the cells were harvest and lysed to release the Target Plasmid. The editing efficiency at RHO-WT and RHO-P23H site was analyzed via NGS with potentially edited Target Plasmid as template, using Target Plasmid specific primer pairs. The results are shown in FIG. 28.

TABLE 10
The exemplary sequences related specific knock out at RHO-P23H site

| Name | SEQ ID NOS | Sequence |
|---|---|---|
| RHO (P23H) | 901 | TGCGACGGGTGTGGTACGCA |
| spacer | | |
| GEBx0308-RHO | 902 | TGCGACGGGTGTGGTACGCAGTTTGAGAACCTTGTAAATTAG |
| (P23H) sgRNA | | AAATAATTTACAAGGTGAGTTCAAATAAGGATTTATCCGAAAT |
| (DNA) | | CGCCGAAATGGCCCACATTGTGTGGATAAAAAAGACTTTATT |
| | | ACAAAGTCTTTTT |
| GEBx0308-RHO | 903 | UGCGACGGGUGUGGUACGCAGUUUGAGAACCUUGUAAAUU |
| (P23H) sgRNA | | AGAAAUAAUUUACAAGGUGAGUUCAAAUAAGGAUUUAUCC |
| (RNA) | | GAAAUCGCCGAAAUGGCCCACAUUGUGUGGAUAAAAAAGA |
| | | CUUUAUUACAAAGUCUUUUU |
| SaCas9-RHO | 904 | ACGGGTGTGGTACGCAGCCACTGTTTTAGTACTCTGGAAACA |
| (P23H) sgRNA | | GAATCTACTAAAACAAGGCAAAATGCCGTGTTTATCTCGTCA |
| (DNA) | | ACTTGTTGGCGAGA |
| SaCas9-RHO | 905 | ACGGGUGUGGUACGCAGCCACUGUUUUAGUACUCUGGAAA |
| (P23H) sgRNA | | CAGAAUCUACUAAAACAAGGCAAAAUGCCGUGUUUAUCUC |
| (RNA) | | GUCAACUUGUUGGCGAGA |
| SaCas9 CDS | 906 | ATGGCCCCAAAGAAGAAGCGGAAGGTCAAGCGGAACTACAT |
| | | CCTGGGCCTGGACATCGGCATCACCAGCGTGGGCTACGGCAT |
| | | CATCGACTACGAGACACGGGACGTGATCGATGCCGGCGTGCG |
| | | GCTGTTCAAAGAGGCCAACGTGGAAAACAACGAGGGCAGGC |
| | | GGAGCAAGAGAGGCGCCAGAAGGCTGAAGCGGCGGAGGCG |
| | | GCATAGAATCCAGAGAGTGAAGAAGCTGCTGTTCGACTACAA |
| | | CCTGCTGACCGACCACAGCGAGCTGAGCGGCATCAACCCCTA |
| | | CGAGGCCAGAGTGAAGGGCCTGAGCCAGAAGCTGAGCGAG |
| | | GAAGAGTTCTCTGCCGCCCTGCTGCACCTGGCCAAGAGAAG |
| | | AGGCGTGCACAACGTGAACGAGGTGGAAGAGGACACCGGC |
| | | AACGAGCTGTCCACCAAAGAGCAGATCAGCCGGAACAGCAA |
| | | GGCCCTGGAAGAGAAATACGTGGCCGAACTGCAGCTGGAAC |
| | | GGCTGAAGAAAGACGGCGAAGTGCGGGGCAGCATCAACAG |
| | | ATTCAAGACCAGCGACTACGTGAAAGAAGCCAAACAGCTGC |
| | | TGAAGGTGCAGAAGGCCTACCACCAGCTGGACCAGAGCTTC |
| | | ATCGACACCTACATCGACCTGCTGGAAACCCGGCGGACCTAC |
| | | TATGAGGGACCTGGCGAGGGCAGCCCCTTCGGCTGGAAGGA |
| | | CATCAAAGAATGGTACGAGATGCTGATGGGCCACTGCACCTA |
| | | CTTCCCCGAGGAACTGCGGAGCGTGAAGTACGCCTACAACG |
| | | CCGACCTGTACAACGCCCTGAACGACCTGAACAATCTCGTGA |
| | | TCACCAGGGACGAGAACGAGAAGCTGGAATATTACGAGAAG |
| | | TTCCAGATCATCGAGAACGTGTTCAAGCAGAAGAAGAAGCC |
| | | CACCCTGAAGCAGATCGCCAAAGAAATCCTCGTGAACGAAG |
| | | AGGATATTAAGGGCTACAGAGTGACCAGCACCGGCAAGCCC |
| | | GAGTTCACCAACCTGAAGGTGTACCACGACATCAAGGACATT |
| | | ACCGCCCGGAAAGAGATTATTGAGAACGCCGAGCTGCTGGAT |
| | | CAGATTGCCAAGATCCTGACCATCTACCAGAGCAGCGAGGAC |
| | | ATCCAGGAAGAACTGACCAATCTGAACTCCGAGCTGACCCA |
| | | GGAAGAGATCGAGCAGATCTCTAATCTGAAGGGCTATACCGG |
| | | CACCCACAACCTGAGCCTGAAGGCCATCAACCTGATCCTGGA |
| | | CGAGCTGTGGCACACCAACGACAACCAGATCGCTATCTTCAA |
| | | CCGGCTGAAGCTGGTGCCCAAGAAGGTGGACCTGTCCCAGC |
| | | AGAAAGAGATCCCCACCACCCTGGTGGACGACTTCATCCTGA |
| | | GCCCCGTCGTGAAGAGAAGCTTCATCCAGAGCATCAAAGTG |
| | | ATCAACGCCATCATCAAGAAGTACGGCCTGCCCAACGACATC |
| | | ATTATCGAGCTGGCCCGCGAGAAGAACTCCAAGGACGCCCA |
| | | GAAAATGATCAACGAGATGCAGAAGCGGAACCGGCAGACCA |
| | | ACGAGCGGATCGAGGAAATCATCCGGACCACCGGCAAAGAG |
| | | AACGCCAAGTACCTGATCGAGAAGATCAAGCTGCACGACATG |
| | | CAGGAAGGCAAGTGCCTGTACAGCCTGGAAGCCATCCCTCTG |
| | | GAAGATCTGCTGAACAACCCCTTCAACTATGAGGTGGACCAC |
| | | ATCATCCCCAGAAGCGTGTCCTTCGACAACAGCTTCAACAAC |
| | | AAGGTGCTCGTGAAGCAGGAAGAAAACAGCAAGAAGGGCA |
| | | ACCGGACCCCATTCCAGTACCTGAGCAGCAGCGACAGCAAG |
| | | ATCAGCTACGAAACCTTCAAGAAGCACATCCTGAATCTGGCC |
| | | AAGGGCAAGGGCAGAATCAGCAAGACCAAGAAAGAGTATCT |
| | | GCTGGAAGAACGGGACATCAACAGGTTCTCCGTGCAGAAAG |
| | | ACTTCATCAACCGGAACCTGGTGGATACCAGATACGCCACCA |
| | | GAGGCCTGATGAACCTGCTGCGGAGCTACTTCAGAGTGAAC |
| | | AACCTGGACGTGAAAGTGAAGTCCATCAATGGCGGCTTCACC |
| | | AGCTTTCTGCGGCGGAAGTGGAAGTTTAAGAAAGAGCGGAA |
| | | CAAGGGGTACAAGCACCACGCCGAGGACGCCCTGATCATTG |
| | | CCAACGCCGATTTCATCTTCAAAGAGTGGAAGAAACTGGACA |
| | | AGGCCAAAAAAGTGATGGAAAACCAGATGTTCGAGGAAAAG |
| | | CAGGCCGAGAGCATGCCCGAGATCGAAACCGAGCAGGAGTA |
| | | CAAAGAGATCTTCATCACCCCCCACCAGATCAAGCACATTAA |
| | | GGACTTCAAGGACTACAAGTACAGCCACCGGGTGGACAAGA |
| | | AGCCTAATAGAGAGCTGATTAACGACACCCTGTACTCCACCC |
| | | GGAAGGACGACAAGGGCAACACCCTGATCGTGAACAATCTG |
| | | AACGGCCTGTACGACAAGGACAATGACAAGCTGAAAAAGCT |
| | | GATCAACAAGAGCCCCGAAAAGCTGCTGATGTACCACCACG |
| | | ACCCCCAGACCTACCAGAAACTGAAGCTGATTATGGAACAGT |
| | | ACGGCGACGAGAAGAATCCCCTGTACAAGTACTACGAGGAA |
| | | ACCGGGAACTACCTGACCAAGTACTCCAAAAAGGACAACGG |
| | | CCCCGTGATCAAGAAGATTAAGTATTACGGCAACAAACTGAA |
| | | CGCCCATCTGGACATCACCGACGACTACCCCAACAGCAGAAA |
| | | CAAGGTCGTGAAGCTGTCCCTGAAGCCCTACAGATTCGACGT |
| | | GTACCTGGACAATGGCGTGTACAAGTTCGTGACCGTGAAGAA |
| | | TCTGGATGTGATCAAAAAAGAAAACTACTACGAAGTGAATAG |
| | | CAAGTGCTATGAGGAAGCTAAGAAGCTGAAGAAGATCAGCA |
| | | ACCAGGCCGAGTTTATCGCCTCCTTCTACAACAACGATCTGAT |
| | | CAAGATCAACGGCGAGCTGTATAGAGTGATCGGCGTGAACAA |
| | | CGACCTGCTGAACCGGATCGAAGTGAACATGATCGACATCAC |
| | | CTACCGCGAGTACCTGGAAAACATGAACGACAAGAGGCCCC |
| | | CCAGGATCATTAAGACAATCGCCTCCAAGACCCAGAGCATTA |
| | | AGAAGTACAGCACAGACATTCTGGGCAACCTGTATGAAGTGA |
| | | AATCTAAGAAGCACCCTCAGATCATCAAAAAGGGCAAAAGG |
| | | CCGGCGGCCACGAAAAAGGCCGGCCAGGCAAAAAAGAAAA |
| | | AGGACTACAAGGACGACGACGACAAGTGA |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. An engineered, non-naturally occurring Type II CRISPR-associated (Cas) protein or a polynucleotide encoding said Cas protein, wherein:
(a) the Cas protein has at least 70%, at least 75%, at least 80%, at least 85%, at least 90%, at least 92%, at least 95%, at least 98%, at least 99%, or 100% sequence identity to the amino acid sequence of SEQ ID NO: 9; or
(b) the Cas protein has at least 70%, at least 75%, at least 80%, at least 85%, at least 90%, at least 92%, at least 95%, at least 98%, at least 99%, or 100% sequence identity to the amino acid sequence of SEQ ID NO: 9, with the exception of the amino acid “M” at position 1 of the sequence.

2. The Cas protein or polynucleotide of claim 1, wherein the Cas protein is capable of recognizing a protospacer adjacent motif (PAM) having a sequence selected from NRRANH.

3. The Cas protein or polynucleotide of claim 1, wherein the Cas protein is a nickase or a dead Cas protein.

4. The Cas protein or polynucleotide of claim 3, wherein:
(a) the Cas protein has at least 70%, at least 75%, at least 80%, at least 85%, at least 90%, at least 92%, at least 95%, at least 98%, at least 99%, or 100% to the amino acid sequence of SEQ ID NO: 9, with a mutation at residue D11 or H859;
(b) the Cas protein has at least 70%, at least 75%, at least 80%, at least 85%, at least 90%, at least 92%, at least 95%, at least 98%, at least 99%, or 100% sequence identity to the amino acid sequence of SEQ ID NO: 9, with the exception of the amino acid “M” at position 1 of the sequence, and comprises a mutation at residue D11 or H859.

5. The Cas protein or polynucleotide of claim 1, wherein the mutation at residue D11 or H859 of SEQ ID NO: 9 is D11A or H859A.

6. The Cas protein or polynucleotide of claim 5, wherein the Cas protein has a sequence identity of at least 70%, at least 75%, at least 80%, at least 85%, at least 90%, at least 92%, at least 95%, at least 98%, at least 99%, or 100% to the amino acid sequence of any one of SEQ ID NOs: 869-871.

7. The Cas protein or polynucleotide of claim 1, wherein the Cas protein further comprises a sequence selected from the group consisting of: a nuclear localization signal sequence, a nuclear export signal sequence, a cell-penetrating peptide sequence, an affinity tag sequence, a deaminase sequence, a reverse transcriptase sequence, a recombinase sequence, a methyltransferase sequence, a methylase sequence, an acetylase sequence, an acetyltransferase sequence, a transcriptional activator sequence, a transcriptional repressor domain sequence, a cryptochrome sequence, a light inducible/controllable domain sequence, and a chemically inducible/controllable domain sequence.

8. The Cas protein or polynucleotide of claim 7, wherein the Cas protein comprises an amino acid sequence having at least 70%, at least 75%, at least 80%, at least 85%, at least 90%, at least 92%, at least 95%, at least 96%, at least 97%, at least 98%, at least 9 9%, or 100% sequence identity to the amino acid sequence of any one of SEQ ID NOs: 89, and 864.

9. The Cas protein or polynucleotide of claim 1, wherein the polynucleotide is a ribonucleotide sequence or a deoxyribonucleotide sequence, or analogs thereof, optionally, the polynucleotide is codon-optimized for expression in a cell of interest; preferably, the polynucleotide is an mRNA and further comprises a 5′ cap sequence and/or a poly-A tail sequence.

10. An engineered, non-naturally occurring CRISPR-Cas system comprising: a) the Type II Cas protein of claim 1 or a polynucleotide encoding the Cas protein thereof; and b) at least one engineered guide RNA or at least one engineered nucleic acid encoding the guide RNA thereof, wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex.

11. The CRISPR-Cas system of claim 10, wherein the guide RNA further comprises a linker sequence connecting the tracrRNA sequence and the DR sequence to form a sgRNA scaffold.

12. The CRISPR-Cas system of claim 11, wherein the sgRNA scaffold comprises a sequence having at least 70%, at least 75%, at least 80%, at least 85%, at least 88%, at least 90%, at least 92%, at least 94%, at least 95%, at least 96%, at least 98%, at least 99% or 100% identity to any one of SEQ ID NOs: 465-472.

13. The CRISPR-Cas system of claim 10, wherein the polynucleotide encoding the Cas protein is operably linked to a promoter; optionally, the promoter is a constitutive promoter, a tissue-specific promoter, or an inducible promoter.

14. The CRISPR-Cas system of claim 13, wherein the polynucleotide encoding the Cas protein is operably linked to a promoter and is present in a vector; optionally, the vector is selected from the group consisting of: a retroviral vector, a lentiviral vector, a phage vector, an adenoviral vector, an adeno-associated virus vector, a herpes simplex virus vector and a plasmid vector.

15. A method of modifying or targeting a target DNA locus, comprising delivering to said target DNA locus with:
(a) the Cas protein or the polynucleotide encoding said Cas protein of claim 1; or
(b) a CRISPR-Cas system; wherein the CRISPR-Cas system comprises a) the Cas protein or the polynucleotide; and b) at least one engineered guide RNA or at least one engineered nucleic acid encoding the guide RNA thereof, wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence that hybridizes to form a double-stranded RNA (dsRNA) duplex.

16. The method of claim 15, wherein said modifying or targeting a target DNA locus comprises inducing a DNA strand break, altering gene expression of one or more genes, or epigenetically modifying of said target DNA locus; optionally, the DNA strand break comprise a DNA double strand break or a DNA single strand break.

17. The method of claim 16, wherein the method is in vitro or in vivo.
